(19) United States
(12) Patent Application Publication
(10) Pub. No.: US 2003/0008841 A1
(43) Pub. Date: Jan. 9, 2003

(54) Anti-HCV nucleoside derivatives

(76) Inventors: Rene Devos, Welwyn Garden City (GB); Brian William Dymock, St. Albans (GB); Christopher John Hobbs, Hertford (GB); Wen-Rong Jiang, Welwyn Garden City (GB); Joseph Armstrong Martin, Harpenden (GB); John Herbert Merrett, Baldock (GB); Isabel Najera, St. Albans (GB); Nobuo Shimma, Chigasaki-shi (JP); Takuo Tsukuda, Odawara-shi (JP)

Correspondence Address: HOFFMANN-LA ROCHE INC., PATENT LAW DEPARTMENT, 340 KINGSLAND STREET, NUTLEY, NJ, 07110

(21) Appl. No.: 09/923,620

(22) Filed: Aug. 7, 2001

(30) Foreign Application Priority Data
Aug. 30, 2000 (GB) 0021285.2
Oct. 31, 2000 (GB) 0026611.4

(51) Int. Cl. A61K 31/712; A61K 31/522; A61K 31/52; A61K 31/513

(52) U.S. Cl. 514/45; 514/263.23; 514/263.37; 514/263.4; 514/49; 514/269; 514/272

(57) ABSTRACT

The present invention comprises novel and known purine and pyrimidine nucleoside derivatives which have been discovered to be active against hepatitis C virus (HCV). The use of these derivatives for the treatment of HCV infection is claimed as are the novel nucleoside derivatives disclosed herein.

### BACKGROUND OF THE INVENTION

Hepatitis C virus is the leading cause of chronic liver disease throughout the world. Patients infected with HCV are at risk of developing cirrhosis of the liver and subsequent hepatocellular carcinoma and hence HCV is the major indication for liver transplantation. Only two approved therapies are currently available for the treatment of HCV infection (R. G. Gish, Sem.Liver.Dis., 1999, 19, 35). These are interferon-α monotherapy and, more recently, combination therapy of the nucleoside analogue, ribavirin (Virazole), with interferon-α.

Ribavirin is a broad spectrum antiviral agent with activity against a range of DNA and RNA viruses (R. A. Smith and W. Kirkpatrick (Eds.): Ribavirin—A Broad Spectrum Antiviral Agent, Academic Press, New York, 1980) but its mechanism of action has not been conclusively established and a number of distinct properties of ribavirin have been identified which may vary in relative importance for differing viral disease conditions. These properties include mediation of the immune response (C. D. Hultgren et al, J.Gen.Virol., 1998, 79, 2381), lowering of serum alanine aminotransferase (ALT) levels (G. Dusheiko et al, J. Hepatol.,1996, 25, 591), inhibition as the monophosphate of inosine monophosphate dehydrogenase

(IMPDH) (D. G. Streeter et al, Proc.Natl.Acad.Sci., 1973, 70,1174) and direct inhibition of viral DNA or RNA replication (R. W. Sidwell et al, Science, 177,705).

Many of the drugs approved for the treatment of viral infections are nucleosides or nucleoside analogues and most of these nucleoside analogue drugs inhibit viral replication, following conversion to the corresponding triphosphates, through inhibition of the viral polymerase enzymes. This conversion to the triphosphate is commonly mediated by cellular kinases and therefore the direct evaluation of nucleosides as inhibitors of HCV replication is only conveniently carried out using a cell-based assay. For HCV the availability of a true cell-based viral replication assay or animal model of infection is lacking.

Hepatitis C virus belongs to the family of Flaviridae. It is an RNA virus, the RNA genome encoding a large polyprotein which after processing produces the necessary replication machinery to ensure synthesis of progeny RNA. It is believed that most of the non-structural proteins encoded by the HCV RNA genome are involved in RNA replication. Lohmann et al. [V. Lohmann et al., Science, 1999, 285, 110-113] have described the construction of a Human Hepatoma (Huh7) cell line in which subgenomic HCV RNA molecules have been introduced and shown to replicate with high efficiency. It is believed that the mechanism of RNA replication in these cell lines is identical to the replication of the full length HCV RNA genome in infected hepatocytes. The subgenomic HCV cDNA clones used for the isolation of these cell lines have formed the basis for the development of a cell-based assay for identifying nucleoside analogue inhibitors of HCV replication.

### SUMMARY OF THE INVENTION

The invention relates to nucleoside derivatives as inhibitors of HCV Replicon RNA replication. In particular, the invention is concerned with novel and known purine and pyrimidine nucleoside derivatives, their use as inhibitors of subgenomic Hepatitis C Virus (HCV) RNA replication and pharmaceutical compositions of such compounds. For the novel purine and pyrimidine nucleoside derivatives the invention is also concerned with a process for their manufacture, pharmaceutical compositions and the use of such compounds in medicine. Accordingly, the compounds of this invention may be useful as therapeutic agents for the treatment of HCV infections.

### DETAILED DESCRIPTION OF THE INVENTION

The present invention concerns compounds of formula I:

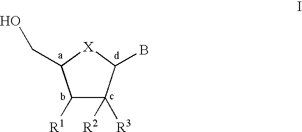

wherein

R¹ is hydrogen, hydroxy, alkyl, hydroxyalkyl, alkoxy, halogen, cyano, isocyano or azido;

R² is hydrogen, hydroxy, alkoxy, chlorine, bromine or iodine;

R³ is hydrogen; or

R² and R³ together represent ═CH₂; or

R² and R³ represent fluorine;

X is O, S or CH₂;

a, b, c, d denoting asymmetric carbon atoms each of which is substituted with 4 different substituents; and

B is a purine base B1 which is connected through the 9-nitrogen of formula

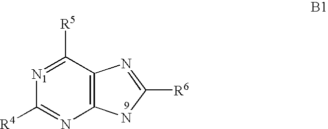

wherein

R⁴ is hydrogen, hydroxy, alkyl, alkoxy, alkylthio, aryloxy, arylthio, heterocyclyl, NR⁷R⁸, halogen or SH;

R⁵ is hydrogen, hydroxy, alkyl, haloalkyl, cycloalkyl, alkoxy, alkylthio, aryl, aryloxy, arylthio, heterocyclyl, heterocyclylamino, halogen, NR⁷R⁸, NHOR⁹, NHNR⁷R⁸ or SH;

R⁶ is hydrogen, hydroxy, alkyl, alkoxy, alkylthio, aryloxy, arylthio, heterocyclyl, NR⁷R⁸, halogen, SH or cyano;

R⁷ and R⁸ are independently of each other hydrogen, alkyl, aryl, hydroxyalkyl, alkenylalkyl, alkynylalkyl, cycloalkyl or acyl;

R⁹ is hydrogen, alkyl or aryl; or

B is an oxidized purine base B2 which is connected through the 9-nitrogen of formula

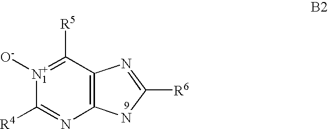

wherein

R⁴, R⁵ and R⁶ are as defined above; or

B is a purine base B3 which is connected through the 9-nitrogen of formula

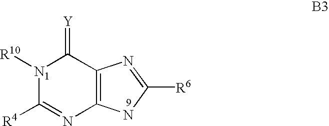

wherein

R⁴ and R⁶ are as defined above;

R¹⁰ is hydrogen, alkyl or aryl;

Y is O, S or NR¹¹;

R¹¹ is hydrogen, hydroxy, alkyl, OR⁹, heterocyclyl or NR⁷R⁸;

R⁷, R⁸ and R⁹ are as defined above; or

B is a pyrimidine base B4 which is connected through the 1-nitrogen of formula

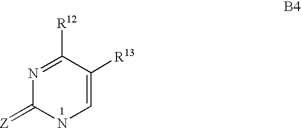

wherein

Z is O or S;

R¹² is hydrogen, hydroxy, alkyl, alkoxy, haloalkyl, alkylthio, aryl, aryloxy, arylthio, heterocyclyl, heterocyclylamino, halogen, NR⁷R⁸, NHOR⁹, NHNR⁷R⁸ or SH;

R¹³ is hydrogen, alkyl, hydroxyalkyl, alkoxyalkyl, haloalkyl, cycloalkyl or halogen;

R⁷, R⁸ and R⁹ are as defined above; or

B is a pyrimidine base B5 which is connected through the 1-nitrogen of formula

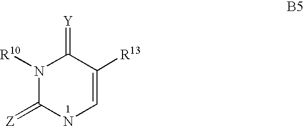

wherein

Y, Z, R¹⁰ and R¹³ are as defined above.

The compounds of Formula I, and optionally the hydrolyzable esters, hydrolyzable ethers and pharmaceutically acceptable salts of such compounds, may be used for the treatment of diseases mediated by the Hepatitis C Virus (HCV) or for the preparation of a medicament for such treatment.

The term “alkyl” as used herein denotes an unsubstituted or substituted straight or branched chain hydrocarbon residue containing 1 to 12 carbon atoms, such as methyl, ethyl, propyl, isopropyl, n-butyl, isobutyl, tert.-butyl, pentyl, hexyl, heptyl, octyl, nonyl, decyl, undecyl, dodecyl including their isomers. Preferably, the term “alkyl” denotes an optionally substituted straight or branched chain hydrocarbon residue containing 1 to 7 carbon atoms.

Suitable substituents for the alkyl chain can be selected from one or more of aryl, heterocyclyl, cycloalkyl, nitro, cyano, azido, amino, alkyl amino, dialkyl amino, cycloalkyl amino, aryl amino, diarylamino, heterocyclyl amino, hydroxy, alkoxy, aryloxy, heterocyclyloxy, cycloalkoxy, thio, alkylthio, arylthio, heterocyclylthio, alkyl carbonyl, cycloalkyl carbonyl, aryl carbonyl, heterocyclyl carbonyl, carboxy, alkoxycarbonyl, aminocarbonyl, alkylaminocarbonyl, arylaminocarbonyl, dialkylaminocarbonyl, diarylaminocarbonyl, heterocyclylaminocarbonyl.

Aryl, heterocyclyl or cycloalkyl as substituents for the alkyl group can also be substituted with one or more methyl, ethyl, n-propyl, i-propyl, tert.-butyl, trifluoromethyl, hydroxy, methoxy, ethoxy, propyloxy, amino, alkylamino, arylamino, dialkylamino, diarylamino, heterocyclylamino,vinyl, allyl, carboxy, alkylcarbonyl, alkoxycarbonyl, aminocarbonyl, alkylaminocarbonyl, arylaminocarbonyl, dialkylaminocarbonyl, diarylaminocarbonyl, heterocyclylaminocarbonyl, fluorine, chlorine, bromine, iodine, cyano or nitro.

Alkyl in R¹ is preferably an unsubstituted straight or branched chain hydrocarbon residue containing 1 to 7 carbon atoms and most preferred methyl, ethyl, propyl, isopropyl, n-butyl, isobutyl, tert.-butyl or pentyl.

Alkyl in R⁴ is preferably an unsubstituted or substituted straight or branched chain hydrocarbon residue containing 1 to 7 carbon atoms. Suitable substituents for the alkyl group are selected from one or more of aryl or heterocyclyl as defined below. The aryl or heterocyclyl can also be alkylated with one or more methyl or ethyl or halogenated with fluorine, chlorine, bromine or iodine. Preferably alkyl in R⁴ is methyl, ethyl, propyl, isopropyl, n-butyl, isobutyl, tert.-butyl, phenylmethyl (benzyl), chlorphenylmethyl, phenylethyl, phenylpropyl, pyridylmethyl, chlorpyridylmethyl, pyridylethyl, pyridylpropyl, thienylmethyl, thienylethyl, thienylpropyl.

Alkyl in R⁵ is preferably an unsubstituted or substituted straight or branched chain hydrocarbon residue containing 1 to 7 carbon atoms. Suitable substituents for the alkyl group are selected from one or more of aryl or heterocyclyl as defined below. The aryl or heterocyclyl can also be alkylated with one or more methyl or ethyl or halogenated with fluorine, chlorine, bromine or iodine. Preferably alkyl in R⁵ is methyl, ethyl, propyl, isopropyl, n-butyl, isobutyl, tert.-butyl, phenylmethyl (benzyl), chlorphenylmethyl, 1-phenylethyl, 2-phenylethyl, phenylpropyl, pyridylmethyl, chlorpyridylmethyl, pyridylethyl, pyridylpropyl, thienylmethyl, thienylethyl, thienylpropyl.

Alkyl in R⁶ is preferably an unsubstituted or substituted straight or branched chain hydrocarbon residue containing 1 to 7 carbon atoms. Suitable substituents for the alkyl group are selected from one or more of hydroxy, aryl or heterocyclyl as defined below. The aryl or heterocyclyl can also be alkylated with one or more methyl or ethyl or halogenated with fluorine, chlorine, bromine or iodine. Preferably alkyl in R⁶ is methyl, ethyl, propyl, isopropyl, n-butyl, isobutyl, tert.-butyl, hydroxymethyl, 1-hydroxyethyl, 2-hydroxyethyl, hydroxypropyl, 1-hydroxy-1-methyl-ethyl, 2-hydroxy-2-methyl-ethyl, phenylmethyl (benzyl), chlorphenylmethyl, phenylethyl, phenylpropyl, pyridylmethyl, chlorpyridylmethyl, pyridylethyl, pyridylpropyl, thienylmethyl, thienylethyl, thienylpropyl.

Alkyl in R⁷ and R⁸ (for NR⁷R⁸) is independently of each other preferably an unsubstituted or substituted straight or branched chain hydrocarbon residue containing 1 to 12 carbon atoms. Suitable substituents for the alkyl group are selected from one or more of aryl, heterocyclyl, cycloalkyl, nitro, amino, alkyl amino, dialkyl amino, cycloalkyl amino, aryl amino, heterocyclyl amino, alkyl carbonyl, cycloalkyl carbonyl, aryl carbonyl, heterocyclyl carbonyl. The aryl, heterocyclyl or cycloalkyl can also be substituted with one or more methyl, ethyl, n-propyl, i-propyl, tert.-butyl, trifluoromethyl, methoxy, ethoxy, propyloxy, amino, vinyl, allyl, carboxy, alkylcarbonyl, fluorine, chlorine, bromine, iodine or aminosulphonyl. Preferably alkyl in R⁷ and R⁸ is methyl, ethyl, propyl, isopropyl, n-butyl, isobutyl, tert.-butyl, pentyl, hexyl, heptyl, aminomethyl, aminoethyl, aminopropyl, aminobutyl, aminopentyl, chlormethyl, chlorethyl, chlorpropyl, cyanomethyl, cyanoethyl, cyanopropyl, phenylmethyl (benzyl), 1-phenylethyl, 2-phenylethyl, 1(S)-methyl-2-phenylethyl, 1(R)-methyl-2-phenylethyl, 1-phenylpropyl, 2-phenylpropyl, 3-phenylpropyl, phenylbutyl, phenylpentyl, phenylhexyl, 1-benzyl-1-methylethyl, chlorphenylmethyl, dichlorphenylmethyl, 2-chlorphenylethyl, 3-chlorphenylethyl, 4-chlorphenylethyl, dichlorphenylethyl, tolylmethyl, tolylethyl, tolylpropyl, tolylbutyl, methoxyphenylmethyl, methoxyphenylethyl, methoxyphenylpropyl, methoxyphenylbutyl, aminophenylmethyl, aminophenylethyl, aminophenylpropyl, aminophenylbutyl, phenolmethyl, phenolethyl, phenolpropyl, phenolbutyl, naphthylmethyl, naphthylethyl, naphthylpropyl, naphthylbutyl, 2-pyridylmethyl, 3-pyridylmethyl, 4-pyridylmethyl, pyridylethyl, pyridylpropyl, methylpyridylmethyl, methylpyridylethyl, methylpyridylpropyl, chlorpyridylmethyl, chlorpyridylethyl, chlorpyridylpropyl, pyrrolylmethyl, pyrrolylethyl, pyrrolylpropyl, pyrrolylbutyl, methylpyrrolylmethyl, methylpyrrolylethyl, methylpyrrolylpropyl, methylpyrrolylbutyl, imidazolylmethyl, imidazolylethyl, imidazolylpropyl, imidazolylbutyl, 2-(3-indolyl)methyl, 2-(3-indolyl)ethyl, 2-(3-indolyl)propyl, morpholinylmethyl, morpholinylethyl, morpholinylpropyl, morpholinylbutyl, thienylmethyl, thienylethyl, 2-(2-thienyl)ethyl, thienylpropyl, thienylbutyl, cyclohexylmethyl, 1-cyclohexylethyl, 2-cyclohexylethyl, cyclohexylpropyl, cyclohexylbutyl, 2-(4-cyanomethylphenyl)ethyl, 2-(3,4-dimethoxyphenyl)ethyl, 2-(4-hydroxyphenyl)ethyl, (5-chloro-2-methoxyphenyl)methyl, (2-methylphenyl)methyl, (3-methyl)butyl, 4-(aminophenyl)methyl, 2-(4-morpholinyl)ethyl, 2(R,S)-phenylpropyl, 2-(4-Methylphenyl)ethyl, 2-(1-methyl-2-pyrrolyl)ethyl, 2-(4-aminosulphonylphenyl)ethyl, 2-ethyl-4-imidazolyl, methyl-1-naphthyl, 2-(4-chlorophenyl)ethyl, 2-(2,4-dichlorophenyl)ethyl, 4-fluorobenzyl, 4-(hydroxycarbonyl)benzyl, 4-trifluoromethyl)benzyl, 2,5-dimethoxy)benzyl, 2-(2-thienyl)ethyl, 2-(4-aminophenyl)ethyl, 2-Phenoxyethyl, (2-thienyl)methyl, 4-(tert-Butyl)benzyl, 1(R)-Phenylethyl, 1(S)-Phenylethyl, 2-Hydroxy-1(S)-phenyl)ethyl.

Alkyl in R⁹ (for NHOR⁹) is preferably an unsubstituted or substituted straight or branched chain hydrocarbon residue containing 1 to 12 carbon atoms such as methyl, ethyl, propyl, isopropyl, n-butyl, isobutyl, tert.-butyl, pentyl, hexyl, heptyl, octyl, nonyl, decyl, undecyl, dodecyl including their isomers. A suitable substituent for the alkyl group is the aryl group as defined below. The aryl can also be substituted with one or more methyl, ethyl, trifluoromethyl, methoxy, ethoxy, hydroxy, amino, fluorine, chlorine, bromine or iodine. Preferred alkyl in R⁹ is methyl, ethyl, propyl, isopropyl, n-butyl, isobutyl, tert.-butyl, pentyl, phenylmethyl (benzyl), phenylethyl, phenylpropyl, phenylbutyl, chlorphenylmethyl, chlorphenylethyl, tolylmethyl, tolylethyl, tolylpropyl, methoxyphenylmethyl, methoxyphenylethyl, aminophenylmethyl, aminophenylethyl, phenolmethyl, phenolethyl.

Alkyl in R¹⁰ is preferably an unsubstituted or substituted straight or branched chain hydrocarbon residue containing 1 to 12 carbon atoms such as methyl, ethyl, propyl, isopropyl, n-butyl, isobutyl, tert.-butyl, pentyl, hexyl, heptyl, octyl, nonyl, decyl, undecyl, dodecyl including their isomers. A suitable substituent for the alkyl group is the aryl group as defined below. The aryl can also be substituted with one or more methyl, ethyl, trifluoromethyl, methoxy, ethoxy, hydroxy, amino, fluorine, chlorine, bromine, iodine.

Preferred alkyl in R¹⁰ is methyl, ethyl, propyl, isopropyl, n-butyl, isobutyl, tert.-butyl, pentyl, phenylmethyl (benzyl), phenylethyl, phenylpropyl, phenylbutyl, chlorphenylmethyl, chlorphenylethyl, tolylmethyl, tolylethyl, tolylpropyl, methoxyphenylmethyl, methoxyphenylethyl, aminophenylmethyl, aminophenylethyl, phenolmethyl, phenolethyl.

Alkyl in R¹¹ is preferably an unsubstituted or substituted straight or branched chain hydrocarbon residue containing 1 to 7 carbon atoms. A suitable substituent for the alkyl group is the aryl group as defined below. The aryl can also be substituted with one or more methyl, ethyl, trifluoromethyl, methoxy) ethoxy, hydroxy, amino, fluorine, chlorine, bromine, iodine. Most preferred alkyl in R¹¹ is methyl, ethyl, propyl, isopropyl, n-butyl, isobutyl, tert.-butyl, pentyl, phenylmethyl (benzyl), phenylethyl, phenylpropyl, phenylbutyl, chlorphenylmethyl, chlorphenylethyl, tolylmethyl, tolylethyl, tolylpropyl, methoxyphenylmethyl, methoxyphenylethyl, aminophenylmethyl, aminophenylethyl, phenolmethyl, phenolethyl.

Alkyl in R¹² is preferably an unsubstituted straight or branched chain hydrocarbon residue containing 1 to 7 carbon atoms and most preferred methyl, ethyl, propyl, isopropyl, n-butyl, isobutyl, tert.-butyl or pentyl.

Alkyl in R¹³ is preferably an unsubstituted or substituted straight or branched chain hydrocarbon residue containing 1 to 7 carbon atoms such as methyl, ethyl, propyl, isopropyl, n-butyl, isobutyl, tert.-butyl or pentyl, hexyl or heptyl. Suitable substituents for the alkyl group are selected from one or more of aryl, heterocyclyl, alkoxy or amino. The aryl or heterocyclyl can also be substituted with one or more methyl, trifluoromethyl, methoxy or amino. Preferably alkyl in R¹³ is methyl, ethyl, propyl, isopropyl, n-butyl, isobutyl, tert.-butyl, pentyl, hexyl, heptyl, methoxymethyl, ethoxymethyl, aminomethyl, aminoethyl, aminopropyl, aminobutyl, phenylmethyl (benzyl), phenylethyl, tolylmethyl, tolylethyl, methoxyphenylmethyl, methoxyphenylethyl, aminophenylmethyl, aminophenylethyl, phenolmethyl, phenolethyl, pyridylmethyl, pyridylethyl, methylpyridylmethyl, pyrrolylmethyl, pyrrolylethyl, methylpyrrolylmethyl, methylpyrrolylethyl, imidazolylmethyl, imidazolylethyl, thienylmethyl, thienylethyl.

The term “cycloalkyl” as used herein denotes an optionally substituted cycloalkyl group containing 3 to 7 carbon atoms, e.g. cyclopropyl, cyclobutyl, cyclopentyl, cyclohexyl or cycloheptyl, which can also be fused to an optionally substituted saturated, partially unsaturated or aromatic monocyclic, bicyclic or tricyclic heterocycle or carbocycle, e.g. to phenyl.

Suitable substituents for cycloalkyl can be selected from one or more of those named for alkyl.

Cycloalkyl in R⁵ is preferably an optionally substituted cycloalkyl group containing 3 to 7 carbon atoms, e.g. cyclopropyl, cyclobutyl, cyclopentyl, cyclohexyl or cycloheptyl. Suitable substituents for the cycloalkyl group are selected from aryl, heterocyclyl, cycloalkyl, hydroxy, nitro, halogen, amino, alkyl amino, dialkyl amino, cycloalkyl amino, aryl amino, heterocyclyl amino. The aryl or heterocyclyl can also be substituted with one or more of methyl, ethyl, trifluoromethyl, methoxy, amino, hydroxy, carboxy, fluorine, chlorine, bromine or iodine. Preferably cycloalkyl in R⁵ is cyclopropyl, cyclobutyl, cyclopentyl, cyclohexyl, cycloheptyl or cyclohexyl substituted with one or more aryl, heterocyclyl, methyl, amino, hydroxy, fluorine or chlorine.

Cycloalkyl in R⁷ and R⁸ (for NR⁷R⁸) is independently of each other preferably an optionally substituted cycloalkyl group containing 3 to 7 carbon atoms, e.g. cyclopropyl, cyclobutyl, cyclopentyl, cyclohexyl or cycloheptyl. Suitable substituents for the cycloalkyl group are selected from aryl, heterocyclyl, cycloalkyl, hydroxy, nitro, halogen, amino, alkyl amino, dialkyl amino, cycloalkyl amino, aryl amino, heterocyclyl amino. The aryl or heterocyclyl can also be substituted with one or more of methyl, ethyl, trifluoromethyl, methoxy, amino, hydroxy, carboxy, fluorine, chlorine, bromine or iodine. Preferably cycloalkyl in R⁷ and R⁸ is cyclopropyl, cyclobutyl, cyclopentyl, cyclohexyl, cycloheptyl or cyclohexyl substituted with one or more aryl, heterocyclyl, methyl, amino, hydroxy, fluorine or chlorine.

Cycloalkyl in R¹³ is preferably an optionally substituted cycloalkyl group containing 3 to 7 carbon atoms, e.g. cyclopropyl, cyclobutyl, cyclopentyl, cyclohexyl or cycloheptyl. Suitable substituents for the cycloalkyl group are selected from one or more of aryl, heterocyclyl, cycloalkyl, hydroxy, nitro, halogen, amino, alkyl amino, dialkyl amino, cycloalkyl amino, aryl amino or heterocyclyl amino. The aryl or heterocyclyl can also be substituted with one or more of methyl, ethyl, trifluoromethyl, methoxy, amino, hydroxy, carboxy, fluorine, chlorine, bromine or iodine. Preferably cycloalkyl in R¹³ is cyclopropyl, cyclobutyl, cyclopentyl, cyclohexyl, cycloheptyl or cyclohexyl substituted with one or more of aryl, heterocyclyl, methyl, amino, hydroxy, fluorine or chlorine.

The term “alkoxy” as used herein denotes an optionally substituted straight or branched chain alkyl-oxy group wherein the “alkyl” portion is as defined above such as methoxy, ethoxy, n-propyloxy, i-propyloxy, n-butyloxy, i-butyloxy, tert.-butyloxy, pentyloxy, hexyloxy, heptyloxy including their isomers.

Suitable substituents for the alkoxy group are selected from aryl, hydroxy, halogen or amino.

Alkoxy in R¹ is preferably an optionally substituted straight or branched chain alkyl-oxy group such as methoxy, ethoxy, n-propyloxy, i-propyloxy, n-butyloxy, i-butyloxy, tert.-butyloxy. Suitable substituents for the alkoxy group are selected from one ore more of aryl, halogen or amino. Preferably alkoxy in R¹ is methoxy, ethoxy, n-propyloxy, i-propyloxy, n-butyloxy, i-butyloxy, tert.-butyloxy, phenylmethoxy, tolylmethoxy, fluormethoxy, chlormethoxy, bromomethoxy, fluorethoxy, chlorethoxy, bromomethoxy, aminomethoxy, aminoethoxy, aminopropyloxy.

Alkoxy in R² is preferably an optionally substituted straight or branched chain alkyl-oxy group such as methoxy, ethoxy, n-propyloxy, i-propyloxy, n-butyloxy, i-butyloxy, tert.-butyloxy. Suitable substituents for the alkoxy group are selected from one ore more of aryl, halogen or amino. Preferably alkoxy in R² is methoxy, ethoxy, n-propyloxy, i-propyloxy, n-butyloxy, i-butyloxy, tert.-butyloxy, phenylmethoxy, tolylmethoxy, fluormethoxy, chlormethoxy, bromomethoxy, fluorethoxy, chlorethoxy, bromomethoxy, aminomethoxy, aminoethoxy, aminopropyloxy.

Alkoxy in R⁴ is preferably an optionally substituted straight or branched chain alkyl-oxy group such as methoxy, ethoxy, n-propyloxy, i-propyloxy, n-butyloxy, i-butyloxy, tert.-butyloxy. Suitable substituents for the alkoxy group are selected from one ore more of aryl, halogen or amino. Preferably alkoxy in R⁴ is methoxy, ethoxy, n-propyloxy, i-propyloxy, n-butyloxy, i-butyloxy, tert.-butyloxy, phenylmethoxy, tolylmethoxy, fluormethoxy, chlormethoxy, bromomethoxy, fluorethoxy, chlorethoxy, bromomethoxy, aminomethoxy, aminoethoxy, aminopropyloxy.

Alkoxy in R⁵ is preferably an optionally substituted straight or branched chain alkyl-oxy group such as methoxy, ethoxy, n-propyloxy, i-propyloxy, n-butyloxy, i-butyloxy, tert.-butyloxy. Suitable substituents for the alkoxy group are selected from one ore more of aryl, halogen or amino. Preferably alkoxy in R⁵ is methoxy, ethoxy, n-propyloxy, i-propyloxy, n-butyloxy, i-butyloxy, tert.-butyloxy, phenylmethoxy, tolylmethoxy, fluormethoxy, chlormethoxy, bromomethoxy, fluorethoxy, chlorethoxy, bromomethoxy, aminomethoxy, aminoethoxy, aminopropyloxy.

Alkoxy in R⁶ is preferably an optionally substituted straight or branched chain alkyl-oxy group such as methoxy, ethoxy, n-propyloxy, i-propyloxy, n-butyloxy, i-butyloxy, tert.-butyloxy. Suitable substituents for the alkoxy group are selected from one ore more of aryl, halogen or amino. Preferably alkoxy in R⁶ is methoxy, ethoxy, n-propyloxy, i-propyloxy, n-butyloxy, i-butyloxy, tert.-butyloxy, phenylmethoxy, tolylmethoxy, fluormethoxy, chlormethoxy, bromomethoxy, fluorethoxy, chlorethoxy, bromomethoxy, aminomethoxy, aminoethoxy, aminopropyloxy.

Alkoxy in R¹² is preferably an optionally substituted straight or branched chain alkyl-oxy group such as methoxy, ethoxy, n-propyloxy, i-propyloxy, n-butyloxy, i-butyloxy, tert.-butyloxy. Suitable substituents for the alkoxy group are selected from one ore more of aryl, halogen or amino. Preferably alkoxy in R¹² is methoxy, ethoxy, n-propyloxy, i-propyloxy, n-butyloxy, i-butyloxy, tert.-butyloxy, phenylmethoxy, tolylmethoxy, fluormethoxy, chlormethoxy, bromomethoxy, fluorethoxy, chlorethoxy, bromomethoxy, aminomethoxy, aminoethoxy, aminopropyloxy.

The term “alkoxyalkyl” as used herein denotes an alkoxy group as defined above which is bonded to an alkyl group as defined above. Examples are methoxymethyl, methoxyethyl, methoxypropyl, ethoxymethyl, ethoxyethyl, ethoxypropyl, propyloxypropyl, methoxybutyl, ethoxybutyl, propyloxybutyl, butyloxybutyl, tert.-butyloxybutyl, methoxypentyl, ethoxypentyl, propyloxypentyl, butyloxypentyl, tert.-butyloxypentyl, pentyloxypentyl, methoxyhexyl, ethoxyhexyl, propyloxyhexyl, butyloxyhexyl, tert.-butyloxyhexyl, pentyloxyhexyl, hexyloxyhexyl, methoxyheptyl, ethoxyheptyl, propyloxyheptyl, butyloxyheptyl, tert.-butyloxyheptyl, pentyloxyheptyl, hexyloxyheptyl, heptyloxyheptyl including their isomers.

Alkoxyalkyl in R¹³ is preferably methoxymethyl, methoxyethyl, methoxypropyl, ethoxymethyl, ethoxyethyl, ethoxypropyl.

The term “alkenyl” as used herein denotes to unsubstituted or substituted hydrocarbon chain radical having from 2 to 7 carbon atoms, preferably from 2 to 4 carbon atoms, and having one or two olefinic double bonds, preferably one olefinic double bond.

Examples are vinyl, 1-propenyl, 2-propenyl (allyl) or 2-butenyl (crotyl).

The term “alkenylalkyl” as used herein denotes an alkenyl group as defined above which is bonded to an alkyl group as defined above. Examples are vinylmethyl (e.g. 1-propenyl or 2-propenyl), 1-propenylmethyl, 2-propenylmethyl or 2-butenylmethyl.

Alkenylalkyl in R⁷ and R⁸ (for NR⁷R⁸) is independently of each other preferably 1-propenyl, 2-propenyl, 1-propenylmethyl or 2-propenylmethyl.

The term “alkynyl” as used herein denotes to unsubstituted or substituted hydrocarbon chain radical having from 2 to 7 carbon atoms, preferably 2 to 4 carbon atoms, and having one or where possible two triple bonds, preferably one triple bond. Examples are ethynyl, 1-propynyl, 2-propynyl, 1-butynyl, 2-butynyl or 3-butynyl.

The term “alkynylalkyl” as used herein denotes an alkynyl group as defined above which is bonded to an alkyl group as defined above. Examples are ethynylmethyl, 1-propynylmethyl, 2-propynylmethyl, 1-butynylmethyl, 2-butynylmethyl or 3-butynylmethyl.

Alkynylalkyl in R⁷ and R⁸ (for NR⁷R⁸) is independently of each other preferably ethynylmethyl, 1-propynylmethyl or 2-propynylmethyl.

The term “hydroxyalkyl” as used herein denotes a straight or branched chain alkyl group as defined above wherein 1, 2, 3 or more hydrogen atoms are substituted by a hydroxy group. Examples are hydroxymethyl, 1-hydroxyethyl, 2-hydroxyethyl, 1-hydroxypropyl, 2-hydroxypropyl, 3-hydroxypropyl, hydroxyisopropyl, hydroxybutyl, hydroxy-isobutyl, hydroxy-tert.-butyl, hydroxypentyl, hydroxyhexyl, hydroxyheptyl and the like.

Hydroxyalkyl in R¹, R⁷, R⁸, R¹³ is preferably hydroxymethyl, 1-hydroxyethyl, 2-hydroxyethyl, hydroxypropyl, hydroxy-isopropyl, hydroxybutyl, hydroxy-isobutyl, hydroxy-tert.-butyl, hydroxypentyl, hydroxyhexyl, hydroxyheptyl and preferred hydroxymethyl, 1-hydroxyethyl, 2-hydroxyethyl, 1-hydroxypropyl, 1-propanol, 2-propanol, 1-butanol, 2-butanol.

The term “haloalkyl” as used herein denotes a straight or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl and the like.

Haloalkyl in R⁵, R¹² and R¹³ is preferably-1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “alkylthio” as used herein denotes a straight or branched chain (alkyl)S-group wherein the “alkyl” portion is as defined above and can be therefore as well substituted with substituents selected from one or more aryl or heterocyclyl. Examples are methylthio, ethylthio, n-propylthio, i-propylthio, n-butylthio, i-butylthio, tert.-butylthio, pentylthio, hexylthio, heptylthio, phenylmethylthio, phenylethylthio, phenylpropylthio, tolylmethylthio, tolylethylthio, tolylpropylthio, pyridylmethylthio, pyridylethylthio, pyridpropylthio, pyrrolylmethylthio, pyrrolylethylthio or pyrrolylpropylthio.

Alkylthio in R⁴, R⁵, R⁶ and R¹² is preferably methylthio, ethylthio, n-propylthio, i-propylthio, n-butylthio, i-butylthio, tert.-butylthio, pentylthio, hexylthio, heptylthio, phenylmethylthio, phenylethylthio, phenylpropylthio, phenylbutylthio, tolylmethylthio, tolylethylthio, tolylpropylthio, pyridylmethylthio, pyridylethylthio, pyridpropylthio, pyrrolylmethylthio, pyrrolylethylthio or pyrrolylpropylthio. Preferred alkylthio in R⁴, R⁵, R⁶ and R¹² is methylthio, ethylthio, n-propylthio, i-propylthio, phenylmethylthio, phenylethylthio, phenylpropylthio, tolylmethylthio, tolylethylthio, pyridylmethylthio, pyridylethylthio) pyrrolylmethylthio or pyrrolylethylthio.

The term “aryl” as used herein denotes an optionally substituted phenyl and naphthyl (e.g. 1-naphthyl, 2-naphthyl or 3-naphthyl), both optionally benz-fused to an optionally substituted saturated, partially unsaturated or aromatic monocyclic, bicyclic or tricyclic heterocycle or carbocycle e.g. to cyclohexyl or cyclopentyl such as 1,2-didehydronaphthyl, 1,2,3,4-tetradehydronaphthyl, anthryl, 1,2-didehydroanthryl, 1,2,3,4-tetradehydroanthryl, phenanthrenyl (e.g. 9-phenanthrenyl), 1,2-didehydrophenanthrenyl or 1,2,3,4-tetradehydrophenanthrenyl.

Suitable substituents for aryl can be selected from those named for alkyl, in addition however, halogen, hydroxy and optionally substituted alkyl, haloalkyl, alkenyl, alkynyl and aryloxy are substituents which can be added to the selection.

Examples for suitable aryls are tolyl, naphthyl (e.g. 1-naphthyl, 2-naphthyl or 3-naphthyl), p-ethylphenyl, p-propylphenyl, p-(i)propylphenyl, p-butylphenyl, p-(i)butylphenyl, p-(t)butylphenyl, 4-(2-methylpropyl)phenyl, p-hydroxyphenyl, p-fluorophenyl, p-chlorophenyl, p-bromophenyl, p-iodophenyl, p-methoxyphenyl, p-ethoxyphenyl, p-methylthiophenyl, p-perfluoromethylphenyl, p-perfluoromethoxyphenyl, biphenyl (e.g. 3-biphenylyl or 4-biphenylyl), p-phenoxyphenyl, m-ethylphenyl, m-propylphenyl, m-(i)propylphenyl, m-butylphenyl, m-(i)butylphenyl, m-(t)butylphenyl, m-hydroxyphenyl, m-fluorophenyl, m-chlorophenyl, m-bromophenyl, m-iodophenyl, m-methoxyphenyl, m-ethoxyphenyl, m-methylthiophenyl, m-perfluoromethylphenyl, m-perfluoromethoxyphenyl, m-phenoxyphenyl, o-ethylphenyl, o-propylphenyl, o-(i)propylphenyl, o-butylphenyl, o-(i)butylphenyl, o-(t)butylphenyl, o-hydroxyphenyl, o-fluorophenyl, o-chlorophenyl, o-bromophenyl, o-iodophenyl, o-methoxyphenyl, o-ethoxyphenyl, o-methylthiophenyl, p-methylthiophenyl, o-perfluoromethylphenyl, o-perfluoromethoxyphenyl or o-phenoxyphenyl. Aryl in R⁵ is preferably phenyl, naphthyl (e.g. 1-naphthyl, 2-naphthyl or 3-naphthyl), tolyl, phenanthrenyl (e.g. 9-phenanthrenyl), p-ethylphenyl, p-propylphenyl, p-(i)propylphenyl, p-butylphenyl, p-(i)butylphenyl, p-(t)butylphenyl, 4-(2-methylpropyl)phenyl, p-hydroxyphenyl, p-fluorophenyl, p-chlorophenyl, p-bromophenyl, p-iodophenyl, p-methoxyphenyl, p-ethoxyphenyl, p-methylthiophenyl, p-perfluoromethylphenyl, p-perfluoromethoxyphenyl, 3-biphenylyl, 4-biphenylyl, p-phenoxyphenyl, m-ethylphenyl, m-propylphenyl, m-(i)propylphenyl, m-butylphenyl, m-(i)butylphenyl, m-(t)butylphenyl, m-hydroxyphenyl, m-fluorophenyl, m-chlorophenyl, m-bromophenyl, m-iodophenyl, m-methoxyphenyl, m-ethoxyphenyl, m-methylthiophenyl, m-perfluoromethylphenyl, m-perfluoromethoxyphenyl, m-phenoxyphenyl, o-ethylphenyl, o-propylphenyl, o-(i)propylphenyl, o-butylphenyl, o-(i)butylphenyl, o-(t)butylphenyl, o-hydroxyphenyl, o-fluorophenyl, o-chlorophenyl, o-bromophenyl, o-iodophenyl, o-methoxyphenyl, o-ethoxyphenyl, o-methylthiophenyl, o-perfluoromethylphenyl, o-perfluoromethoxyphenyl or o-phenoxyphenyl.

Aryl in R⁵, R⁷, R⁸, R⁹, R¹⁰ and R¹² is preferably tolyl, p-ethylphenyl, p-hydroxyphenyl, p-fluorophenyl, p-chlorophenyl, p-bromophenyl, p-iodophenyl, p-methoxyphenyl, p-ethoxyphenyl, p-perfluoromethylphenyl, p-perfluoromethoxyphenyl, 4-biphenylyl, p-phenoxyphenyl, m-ethylphenyl, m-hydroxyphenyl, m-fluorophenyl, m-chlorophenyl, m-bromophenyl, m-iodophenyl, m-methoxyphenyl, m-perfluoromethylphenyl, m-perfluoromethoxyphenyl, m-phenoxyphenyl, o-ethylphenyl, o-hydroxyphenyl, o-fluorophenyl, o-chlorophenyl, o-bromophenyl, o-iodophenyl, o-methoxyphenyl, o-ethoxyphenyl, o-methylthiophenyl, o-perfluoromethylphenyl, o-perfluoromethoxyphenyl or o-phenoxyphenyl.

The term “aryloxy” as used herein denotes an aryl group as defined above which is bonded via an oxygen atom. Examples are phenyloxy, naphthyloxy and the like.

Aryloxy in R⁴, R⁵, R⁶ and R¹² is preferably phenyloxy or naphthyloxy, preferred phenyloxy.

The term “arylthio” as used herein denotes an (aryl)S-group wherein the “aryl” portion is as defined above. Examples are phenylthio or naphthylthio.

Arylthio in R⁴, R⁵, R⁶ and R¹² is preferably phenylthio or naphthylthio, preferred phenylthio.

The term “heterocyclyl” as used herein denotes an optionally substituted saturated, partially unsaturated or aromatic monocyclic, bicyclic or tricyclic heterocyclic systems which contain one or more hetero atoms selected from nitrogen, oxygen and sulfur which can also be fused to an optionally substituted saturated, partially unsaturated or aromatic monocyclic carbocycle or heterocycle.

Examples of suitable heterocycles are oxazolyl, isoxazolyl, furyl, tetrahydrofuryl, 1,3-dioxolanyl, dihydropyranyl, 2-thienyl, 3-thienyl, pyrazinyl, isothiazolyl, isoquinolinyl, indolyl, didehydroindolyl, indazolyl, quinolinyl, dihydrooxazolyl, pyrimidinyl, benzofuranyl, tetrazolyl, 1-pyrrolidinyl, 2-pyrrolidinyl, 3-pyrrolidinyl, pyrrolidinonyl, (N-oxide)-pyridinyl, 1-pyrrolyl, 2-pyrrolyl, triazolyl e.g. 1,2,3-triazolyl or 1,2,4-triazolyl, 1-pyrazolyl, 2-pyrazolyl, 4-pyrazolyl, benzotriazolyl, piperidinyl, morpholinyl (e.g. 4-morpholinyl), thiomorpholinyl (e.g. 4-thiomorpholinyl), thiazolyl, pyridinyl, dihydrothiazolyl, imidazolidinyl, pyrazolinyl, benzothienyl, piperazinyl, 1-imidazolyl, 2-imidazolyl, 4-imidazolyl, thiadiazolyl e.g. 1,2,3-thiadiazolyl, 1,2,3,4-tetrahydroquinoline, 1,2,3,4-tetrahydroisoquinoline, benzothiazolyl, thianthrene (e.g. 1-thianthrenyl) or heptamethyleneimine, 1,2,4,5-tetrahydro-3H-benzazepin-3-yl, 1 ,2,3,4-tetrahydro-2-isoquinolyl, 4-methylpiperazinyl, 1,3,4,5-tetrahydro-2H-benzazepin-2-yl, 2,3-dihydro-1i-indolyl, 2-isoindolinyl, 2,3,4,5-tetrahydro-1,4-benzothiazepin-4-yl, 2,3,4,5-tetrahydro-1,4-benzoxazepin-4-yl, 8-aminosulphonyl-2,3,4,5-tetrahydro-1H-2-benzazepin-2-yl, 7-aminosulphonyl-2,3,4,5-tetrahydro-1H-benzazepin-3-yl, 10,11-dihydro-5H-dibenzo[a,d]cyclohepten-5-yl, 1-hexamethyleneimino, 4-hydroxypiperidin-1-yl, 1,2,3,4-tetrahydro-2-isoquinolyl, 4-phenyl-1-piperazinyl.

Suitable substituents for heterocyclyl can be selected from those named for alkyl, in addition however, optionally substituted alkyl, alkenyl, alkynyl, an oxo group (═O) or aminosulphonyl are substituents which can be added to the selection.

Heterocyclyl in R⁴ is preferably unsubstituted or substituted furyl, tetrahydrofuryl, thienyl, indolyl, indazolyl, pyrimidinyl, benzofuranyl, 1-pyrrolidinyl, pyrrolidinonyl, (N-oxide)-pyridinyl, pyrrolyl, piperidinyl, morpholinyl, imidazolyl or benzothiazolyl. Suitable substituents for heterocyclyl in R⁴ can be selected from unsubstituted or substituted alkyl, unsubstituted or substituted aryl, nitro, cyano and amino.

Heterocyclyl in R⁵ is preferably unsubstituted or substituted oxazolyl, isoxazolyl, furyl, tetrahydrofuryl, 1,3-dioxolanyl, dihydropyranyl, thienyl, pyrazinyl, isothiazolyl, isoquinolinyl, 1-indolyl, didehydroindolyl, indazolyl, quinolinyl, dihydrooxazolyl, pyrimidinyl, benzofuranyl, tetrazolyl, 1-pyrrolidinyl, pyrrolidinonyl, (N-oxide)-pyridinyl, 1,2,3,6-tetradehydropyridine, 1-pyrrolyl, 2-pyrrolyl, triazolyl e.g. 1,2,4-triazolyl, 1-pyrazolyl, 2-pyrazolyl, benzotriazolyl, piperidinyl, 4-morpholinyl, 4-thiomorpholinyl, thiazolyl, pyridinyl, dihydrothiazolyl, imidazolidinyl, pyrazolinyl, benzothienyl, piperazinyl, 1-imidazolyl, thiadiazolyl e.g. 1,2,3-thiadiazolyl, benzothiazolyl, 1-thianthrenyl or heptamethyleneimine, 1,2,4,5-tetrahydro-3H-benzazepin-3-yl, 1,2,3,4-tetrahydro-2-isoquinolyl, 4-methylpiperazinyl, 1,3,4,5-tetrahydro-2H-benzazepin-2-yl, 2,3-dihydro-1-indolyl, 2-isoindolinyl, 2,3,4,5-tetrahydro-1,4-benzothiazepin-4-yl, 2,3,4,5-tetrahydro-1,4-benzoxazepin-4-yl, 8-aminosulphonyl-2,3,4,5-tetrahydro-1H-2-benzazepin-2-yl, 7-aminosulphonyl-2,3,4,5-tetrahydro-1H-benzazepin-3-yl, 10,11-dihydro-5H-dibenzo [a,d]cyclohepten-5-yl, 1-hexamethyleneimino, 4-hydroxypiperidin-1-yl, 1 ,2,3,4-tetrahydro-2-isoquinolyl, 4-phenyl-1-piperazinyl.

Suitable substituents for heterocyclyl in R⁵ can be selected from unsubstituted or substituted alkyl as defined above, unsubstituted or substituted aryl as defined above, nitro, cyano and amino. Examples for substituted heterocyclyl are methylpiperazinyl, ethylpiperazinyl, propylpiperazinyl, butylpiperazinyl, phenylylpiperazinyl, methoxyphenylylpiperazinyl (e.g. 4-(2-Methoxyphenyl)piperazinyl), ethoxyphenylylpiperazinyl, propyloxyphenylylpiperazinyl, benzo-fused thianthrene or 4-(4-Fluorophenyl)-1,2,5,6-tetrahydropyridyl.

Heterocyclyl in R⁶ is preferably unsubstituted or substituted oxazolyl, isoxazolyl, furyl, tetrahydrofuryl, 1,3-dioxolanyl, dihydropyranyl, 2-thienyl, 3-thienyl, pyrazinyl, isothiazolyl, isoquinolinyl, indolyl, didehydroindolyl, indazolyl, quinolinyl, dihydrooxazolyl, pyrimidinyl, benzofuranyl, tetrazolyl, 1-pyrrolidinyl, 2-pyrrolidinyl, 3-pyrrolidinyl, pyrrolidinonyl, (N-oxide)-pyridinyl, 1,2,3,6-tetradehydropyridine, pyrrolyl, 1,2,3-triazolyl, 1,2,4-triazolyl, 1-pyrazolyl, 2-pyrazolyl, 4-pyrazolyl, benzotriazolyl, 1-piperidinyl, 4-morpholinyl, thiomorpholinyl, thiazolyl, pyridinyl, dihydrothiazolyl, imidazolidinyl, 1-imidazolyl, 2-imidazolyl, 4-imidazolyl, pyrazolinyl, benzothienyl, piperazinyl, imidazolyl, thiadiazolyl e.g. 1,2,3-thiadiazolyl, 1,2,3,4-tetrahydroquinoline, 1,2,3,4-tetrahydroisoquinoline, benzothiazolyl, thianthrene or heptamethyleneimine.

Suitable substituents for heterocyclyl in R⁶ can be selected from unsubstituted or substituted alkyl as defined above, unsubstituted or substituted aryl as defined above, nitro, cyano and amino. Examples for substituted heterocyclyl are methylpiperazinyl, ethylpiperazinyl, propylpiperazinyl, butylpiperazinyl, phenylylpiperazinyl, methoxyphenylylpiperazinyl, ethoxyphenylylpiperazinyl, propyloxyphenylylpiperazinyl or benzo-fused thianthrene.

Heterocyclyl in R¹¹ or R¹² is preferably unsubstituted or substituted furyl, tetrahydrofuryl, thienyl indolyl, indazolyl, pyrimidinyl, benzofuranyl, pyrrolidinyl, pyrrolidinonyl, (N-oxide)-pyridinyl, 1-pyrrolyl, piperidinyl, morpholinyl, imidazolyl or benzothiazolyl. Suitable substituents for heterocyclyl in R⁴ can be selected from unsubstituted or substituted alkyl, unsubstituted or substituted aryl, nitro, cyano and amino.

The term “heterocyclylamino” refers to a group of formula (heterocyclyl)N(H), wherein heterocyclyl is as defined above. Examples are furylamino, tetrahydrofurylamino, dihydropyranylamino, thienylamino, pyrazinylamino, indolylamino, indazolylamino, quinolinylamino, benzofuranylamino, pyrrolidinylamino, pyrrolidinonylamino, (N-oxide)-pyridinylamino, pyrrolylamino, pyrazolylamino, benzotriazolylamino, piperidinylamino, morpholinylamino, thiazolylamino, pyridinylamino, imidazolidinylamino, benzothienylamino, imidazolylamino or benzothiazolylamino.

Heterocyclylamino in R⁵ or R¹² is preferably furylamino, tetrahydrofurylamino, dihydropyranylamino, thienylamino, pyrazinylamino, indolylamino, indazolylamino, quinolinylamino, benzofuranylamino, pyrrolidinylamino, pyrrolidinonylamino, (N-oxide)-pyridinylamino, pyrrolylamino, pyrazolylamino, benzotriazolylamino, piperidinylamino, morpholinylamino, thiazolylamino, pyridinylamino, imidazolidinylamino, benzothienylamino, imidazolylamino or benzothiazolylamino.

The term “acyl” as used herein denotes a group of formula C(—O)R wherein R is hydrogen, an unsubstituted or substituted straight or branched chain hydrocarbon residue containing 1 to 7 carbon atoms or a phenyl group. Most preferred acyl groups are those wherein R is hydrogen, an unsubstituted straight chain or branched hydrocarbon residue containing 1 to 4 carbon atoms or a phenyl group.

Acyl in R⁷ and R⁸ (for NR⁷R⁸) is independently of each other preferably methylcarbonyl (acetyl), ethylcarbonyl (propionyl), propylcarbonyl, butylcarbonyl or phenylcarbonyl (benzoyl).

The term halogen stands for fluorine, chlorine, bromine or iodine, preferable fluorine, chlorine, bromine.

Halogen in R¹ is preferably fluorine, chlorine or iodine and more preferred fluorine.

Halogen in R⁴ is preferably chlorine.

Halogen in R⁵ is preferably chlorine.

Halogen in R⁶ is preferably chlorine or bromine.

Halogen in R¹² or R¹³ is preferably fluorine, chlorine, bromine or iodine, more preferred fluorine, chlorine or bromine.

Within the invention the term “X” represents O, S or CH₂, preferably O or CH₂. Most preferred “X” represents O.

Within the invention the term “Y” represents O, S or NR¹¹, wherein R¹¹ represents hydrogen, hydroxy or alkyl which denotes an unsubstituted or aryl-substituted straight or branched chain hydrocarbon residue containing 1 to 7 carbon atoms. Preferably “Y” represents O, S or NR¹¹ wherein R¹¹ represents hydrogen, hydroxy, phenylmethyl (benzyl), phenylethyl, phenylpropyl, phenylbutyl.

Within the invention the term “Z” represents O or S, more preferred O.

In the pictorial representation of the compounds given throughout this application, a thickened tapered line (▾) indicates a substituent which is above the plane of the ring to which the asymmetric carbon belongs, a dotted line (---) indicates a substituent which is below the plane of the ring to which the asymmetric carbon belongs, and a wavy line (˜) indicates a substituent which can be either above or below the plane of the molecule. It is to be understood that the pictorial representation of the compounds given throughout the specification are set forth for convenience and are to be construed as inclusive of other forms including stereoisomers, enantiomers and racemates and are not to be construed as limited to the particular form shown.

Compounds of formula I exhibit stereoisomerism. The compounds of this invention can be any isomer of the compound of formula I or mixtures of these isomers. The compounds and intermediates of the present invention having one or more asymmetric carbon atoms may be obtained as racemic mixtures of stereoisomers which can be resolved, at the appropriate steps in the process of this invention by methods known in the art to obtain a given stereoisomer or pure enantiomer having a desired stereoconfiguration. Alternatively, the desired isomers may be directly synthesised by methods known in the art.

Asymmetric carbon atoms in the compounds of the present invention are denoted as a, b, c and d. The stereoconfiguration of each of the asymmetric carbon atoms denoted as a, b, c, and d can be designated according to the particular stereoisomer it represents.

Compounds of the present invention include those compounds wherein the carbon atom denoted as “a” has the S, R, or R,S-configuration; the carbon atom denoted as “b” has the S, R, or R,S-configuration; the carbon atom denoted as “c” has the S, R, or R,S-configuration; and the carbon atom denoted as “d” has the S, R, or R,S-configuration. In a preferred embodiment of the invention a, b, c and d denoting asymmetric carbon atoms and forming a α-D, β-D, α-L or β-L ribofuranosyl ring. Preferably a, b, c and d denoting asymmetric carbon atoms and forming an α-D or β-D ribofuranosyl ring and most preferred, β-D ribofuranosyl ring.

Compounds of formula 1 exhibit tautomerism that means that the compounds of this invention can exist as two or more chemical compounds that are capable of facile interconversion. In many cases it merely means the exchange of a hydrogen atom between two other atoms, to either of which it forms a covalent bond. Tautomeric compounds exist in a mobile equilibrium with each other, so that attempts to prepare the separate substances usually result in the formation of a mixture that shows all the chemical and physical properties to be expected on the basis of the structures of the components.

The most common type of tautomerism is that involving carbonyl, or keto, compounds and unsaturated hydroxyl compounds, or enols. The structural change is the shift of a hydrogen atom between atoms of carbon and oxygen, with the rearrangement of bonds as indicated.

For example, in many aliphatic aldehydes and ketones, such as acetaldehyde, the keto form is the predominant one; in phenols, the enol form is the major component. An intermediate situation is represented for example in ethyl acetoacetate, which at room temperature contains about 92.4 percent keto and 7.6 percent enol; at −78° C., the interconversion of the two forms is slow enough for the individual substances to be isolated.

It will be appreciated that within the present invention compounds of formula I exist in various tautomeric forms and that they are encompassed by the present invention.

A preferred embodiment of the invention is the use of compounds of formula I wherein

B is a purine base B1 which is connected through the 9-nitrogen of formula

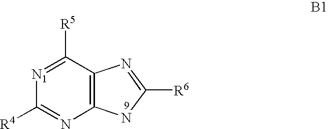

wherein

R⁴, R⁵, R⁶, R⁷, R⁸ and R⁹ are as defined in formula I;

with the proviso that R⁴ is not NH₂ and R⁵ is not NH(CH₃); or

B is a pyrimidine base B4 which is connected through the 1-nitrogen of formula

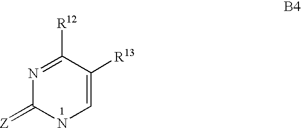

wherein

Z, R⁷, R⁸, R⁹, R¹², R¹³ are as defined in formula I;

with the proviso that R¹² is not hydroxy, alkoxy, N(CH₃)₂, N(H)NH(CH₃) or

N(H)NH₂ and R¹³ is not hydroxyalkyl, chlorine or bromine; or

B is a pyrimidine base B5 which is connected through the 1-nitrogen of formula

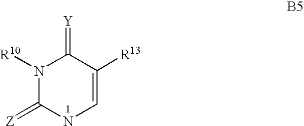

wherein

Y, Z, R¹⁰ and R¹³ are as defined in formula I;

with the proviso that R¹⁰ is not methyl or hydroxyethyl;

for the treatment of diseases mediated by the Hepatitis C Virus (HIV) or for the preparation of a medicament for such treatment.

A further preferred embodiment of the invention is the use of compounds of formula I wherein

R¹ is hydrogen, hydroxy, alkyl, hydroxyalkyl, alkoxy or halogen, preferably wherein

R¹ is hydroxy;

R² is hydrogen, hydroxy, alkoxy, chlorine, bromine or iodine, preferably wherein

R² is hydroxy;

R³ is hydrogen; or

R² and R³ represent fluorine;

X is O;

a, b, c and d denoting asymmetric carbon atoms and forming a D-ribofuranosyl ring, preferably wherein

a, b, c and d denoting asymmetric carbon atoms and forming a β-D-ribofuranosyl ring;

for the treatment of diseases mediated by the Hepatitis C Virus (HIV) or for the preparation of a medicament for such treatment.

A particularly preferred embodiment of the invention is the use of compounds of formula I wherein

B is a purine base B1 which is connected through the 9-nitrogen of formula

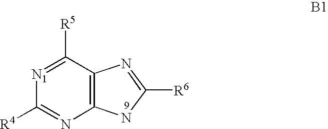

wherein

R⁴ is hydrogen, hydroxy, alkyl, alkoxy, alkylthio, aryloxy, arylthio, heterocyclyl, NR⁷R⁸, halogen or SH, preferably wherein

R⁴ is hydrogen, chlorine or NH₂, most preferred wherein

R⁴ is hydrogen;

R⁵ is hydrogen, hydroxy, alkyl, haloalkyl, cycloalkyl, alkoxy, alkylthio, aryl, aryloxy, arylthio, heterocyclyl, heterocyclylamino, halogen, NR⁷R⁸, NHOR⁹, NHNR⁷R⁸ or SH, preferably wherein

R⁵ is hydroxy, alkylthio, aryl, heterocyclyl, halogen, NR⁷R⁸ or SH, most preferred wherein

R⁵ is alkylthio, aryl, heterocyclyl, halogen or NR⁷R⁸;

R⁶ is hydrogen, hydroxy, alkyl, alkoxy, alkylthio, aryloxy, arylthio, heterocyclyl, NR⁷R⁸, halogen, SH or cyano, preferably wherein

R⁶ is hydrogen, halogen, heterocyclyl or NR⁷R⁸, most preferred wherein

R⁶ is hydrogen or halogen;

R⁷ and R⁸ are independently of each other hydrogen, alkyl, aryl, hydroxyalkyl, alkenylalkyl, alkynylalkyl, cycloalkyl or acyl, preferably wherein

R⁷ and R⁸ are independently of each other hydrogen, alkyl, aryl, alkenylalkyl or alkynylalkyl, most preferred wherein

R⁷ and R⁸ are independently of each other hydrogen, alkyl, alkenylalkyl or alkynylalkyl;

R⁹ is hydrogen, alkyl or aryl;

for the treatment of diseases mediated by the Hepatitis C Virus (HIV) or for the preparation of a medicament for such treatment.

A further preferred embodiment of the invention is the use of compounds of formula I wherein

B is a purine base B1 which is connected through the 9-nitrogen of formula

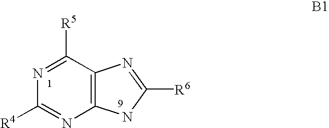

wherein

R⁴ is hydrogen, hydroxy, alkyl, alkoxy, alkylthio, aryloxy, arylthio, heterocyclyl, NR⁷R⁸, halogen or SH, preferably wherein

R⁴ is hydrogen or chlorine, most preferred wherein

R⁴ is hydrogen;

R⁵ is hydrogen, hydroxy, alkyl, haloalkyl, cycloalkyl, alkoxy, alkylthio, aryl, aryloxy, arylthio, heterocyclyl, heterocyclylamino, halogen, NR⁷R⁸, NHOR⁹, NHNR⁷R⁸ or SH, preferably wherein

R⁵ is hydroxy, alkylthio, aryl, heterocyclyl, halogen, NR⁷R⁸ or SH, most preferred wherein

R⁵ is alkylthio, aryl, heterocyclyl, halogen or NR⁷R⁸;

R⁶ is hydrogen, hydroxy, alkyl, alkoxy, alkylthio, aryloxy, arylthio, heterocyclyl, NR⁷R⁸, halogen, SH or cyano, preferably wherein

R⁶ is hydrogen, halogen, heterocyclyl or NR⁷R⁸, most preferred wherein

R⁶ is hydrogen or halogen;

R⁷ and R⁸ are independently of each other hydrogen, alkyl, aryl, hydroxyalkyl, alkenylalkyl, alkynylalkyl, cycloalkyl or acyl, preferably wherein

R⁷ and R⁸ are independently of each other hydrogen, alkyl, aryl, alkenylalkyl or alkynylalkyl;

R⁹ is hydrogen, alkyl or aryl; with the proviso that R⁴ is not NH₂ and R⁵is not NH(CH₃), preferably with the proviso that R⁵ is not NH(CH₃);

for the treatment of diseases mediated by the Hepatitis C Virus (HIV) or for the preparation of a medicament for such treatment.

A particularly preferred embodiment of the invention is the use of compounds of formula I wherein

B is an oxidised purine base B2 which is connected through the 9-nitrogen of formula

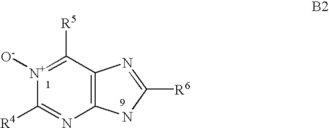

wherein

R⁴ is hydrogen, hydroxy, alkyl, alkoxy, alkylthio, aryloxy, arylthio, heterocyclyl, NR⁷R⁸, halogen or SH, preferably wherein

R⁴ is hydrogen;

R⁵ is hydrogen, hydroxy, alkyl, haloalkyl, cycloalkyl, alkoxy, alkylthio, aryl, aryloxy, arylthio, heterocyclyl, heterocyclylamino, halogen, NR⁷R⁸, NHOR⁹, NHNR⁷R⁸ or SH, preferably wherein

R⁵ is hydrogen, alkyl, heterocyclyl or NR⁷R⁸;

R⁶ is hydrogen, hydroxy, alkyl, alkoxy, alkylthio, aryloxy, arylthio, heterocyclyl, NR⁷R⁸, halogen, SH or cyano, preferably wherein

R⁶ is hydrogen;

R⁷ and R⁸ are independently of each other hydrogen, alkyl, aryl, hydroxyalkyl, alkenylalkyl, alkynylalkyl, cycloalkyl or acyl, preferably wherein

R⁷ and R⁸ are independently of each other hydrogen, alkyl, aryl, hydroxyalkyl, alkenylalkyl, alkynylalkyl, cycloalkyl or acyl;

R⁹ is hydrogen, alkyl or aryl;

for the treatment of diseases mediated by the Hepatitis C Virus (HIV) or for the preparation of a medicament for such treatment.

Another preferred embodiment of the invention is the use of compounds of formula I wherein

B is a purine base B3 which is connected through the 9-nitrogen of formula

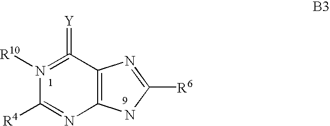

wherein

R⁴ is hydrogen, hydroxy, alkyl, alkoxy, alkylthio, aryloxy, arylthio, heterocyclyl, NR⁷R⁸, halogen or SH, preferably wherein

R⁴ is hydrogen, NR⁷R⁸ or hydroxy;

R⁶ is hydrogen, hydroxy, alkyl, alkoxy, alkylthio, aryloxy, arylthio, heterocyclyl, NR⁷R⁸, halogen, SH or cyano, preferably wherein

R⁶ is hydrogen, halogen or NR⁷R⁸;

R⁷ and R⁸ are independently of each other hydrogen, alkyl, aryl, hydroxyalkyl, alkenylalkyl, alkynylalkyl, cycloalkyl or acyl, preferably wherein

R⁷ and R⁸ are independently of each other hydrogen or alkyl;

R⁹ is hydrogen, alkyl or aryl;

R¹⁰ is hydrogen, alkyl or aryl, preferably wherein

R¹⁰ is hydrogen or alkyl;

Y is O, S or NR¹¹, preferably wherein

Y is O, S, NH or N-alkyl;

R¹¹ is hydrogen, hydroxy, alkyl, OR⁹, heterocyclyl or NR⁷R⁸;

for the treatment of diseases mediated by the Hepatitis C Virus (HIV) or for the preparation of a medicament for such treatment.

Another preferred embodiment of the invention is the use of compounds of formula I wherein

B is a pyrimidine base B4 which is connected through the 1-nitrogen of formula

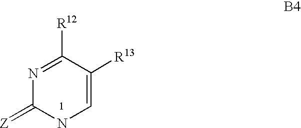

wherein

Z is O or S, preferably wherein

Z is O;

R¹² is hydrogen, hydroxy, alkyl, alkoxy, haloalkyl, alkylthio, aryl, aryloxy, arylthio, heterocyclyl, heterocyclylamino, halogen, NR⁷R⁸, NHOR⁹, NHNR⁷R⁸ or SH, preferably wherein

R¹² is hydroxy, alkyl, heterocyclyl, NR⁷R⁸, NHOR⁹, heterocyclylamino, NHNR⁷R⁸ or SH, most preferred wherein

R¹² is hydroxy, alkyl or NR⁷R⁸;

R¹³ is hydrogen, alkyl, hydroxyalkyl, alkoxyalkyl, haloalkyl, cycloalkyl or halogen, preferably wherein

R¹³ is hydrogen, alkyl or halogen, most preferred wherein

R¹³ is hydrogen;

R⁷ and R⁸ are independently of each other hydrogen, alkyl, aryl, hydroxyalkyl, alkenylalkyl, alkynylalkyl, cycloalkyl or acyl, preferably wherein

R⁷ and R⁸ are independently of each other hydrogen or alkyl;

R⁹ is hydrogen, alkyl or aryl;

for the treatment of diseases mediated by the Hepatitis C Virus (HIV) or for the preparation of a medicament for such treatment.

A further preferred embodiment of the invention is the use of compounds of formula I wherein

B is a pyrimidine base B4 which is connected through the 1-nitrogen of formula

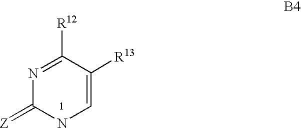

wherein

Z is O or S, preferably wherein

Z is O;

R¹² is hydrogen, alkyl, haloalkyl, alkylthio, aryl, aryloxy, arylthio, heterocyclyl, heterocyclylamino, halogen, NR⁷R⁸, NHOR⁹, NHNR⁷R⁸ or SH, preferably wherein

R¹² is alkyl, heterocyclyl, NR⁷R⁸, NHOR⁹, heterocyclylamino, NHNR⁷R⁸ or SH, most preferred wherein

R¹² is hydroxy, alkyl or NR⁷R;

R¹³ is hydrogen, alkyl, hydroxyalkyl, alkoxyalkyl, haloalkyl, cycloalkyl or halogen, preferably wherein

R¹³ is hydrogen, alkyl or halogen, most preferred wherein

R¹³ is hydrogen;

R⁷ and R⁸ are independently of each other hydrogen, alkyl, aryl, hydroxyalkyl, alkenylalkyl, alkynylalkyl, cycloalkyl or acyl, preferably wherein

R⁷ and R⁸ are independently of each other hydrogen or alkyl;

R⁹ is hydrogen, alkyl or aryl; with the proviso that R¹² is not N(CH₃)₂, N(H)NH(CH₃) or N(H)NH₂ and R¹³ is not hydroxyalkyl, chlorine or bromine, preferably with the proviso that R¹² is not N(CH₃)₂, N(H)NH(CH₃) or N(H)NH₂;

for the treatment of diseases mediated by the Hepatitis C Virus (HIV) or for the preparation of a medicament for such treatment.

Another preferred embodiment of the invention is the use of compounds of formula I wherein

B is a pyrimidine base B5 which is connected through the 1-nitrogen of formula

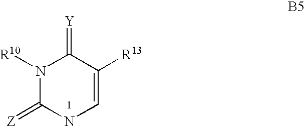

wherein

Y is O, S or NR¹¹, preferably wherein

Y is O or NR¹¹;

Z is O or S, preferably wherein

Z is O;

R¹⁰ is hydrogen, alkyl or aryl, preferably wherein

R¹⁰ is hydrogen;

R¹³ is hydrogen, alkyl, hydroxyalkyl, alkoxyalkyl, haloalkyl, cycloalkyl or halogen, preferably wherein

R¹³ is hydrogen, alkyl or halogen;

for the treatment of diseases mediated by the Hepatitis C Virus (HIV) or for the preparation of a medicament for such treatment.

A further preferred embodiment of the invention is the use of compounds of formula I wherein

R¹ is hydrogen, halogen, hydroxy, alkyl, alkoxy, cyano or azido, preferably wherein

R¹ is hydrogen, fluorine, hydroxy, C₁₋₄-alkyl, C₁₋₄-alkoxy, cyano or azido;

R² is hydrogen or hydroxy; or

R² and R³ represent fluorine;

X is O or CH₂;

a, b, c, d denoting asymmetric carbon atoms each of which is substituted with 4 different substituents; and

B is a pyrimidine base B4 which is connected through the 1-nitrogen of formula

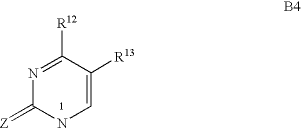

wherein

Z is O;

R¹² is NR⁷R⁸;

R¹³ is hydrogen, alkyl or halogen, preferably wherein

R¹³ is hydrogen, C₁₋₄-alkyl or fluorine;

R⁷ and R⁸ are independently of each other hydrogen or alkyl, preferably wherein

R⁷ and R⁸ are independently of each other hydrogen or C₁₋₄-alkyl; for the treatment of diseases mediated by the Hepatitis C Virus (HIV) or for the preparation of a medicament for such treatment.

Another preferred embodiment of the invention is the use of compounds of formula I wherein

R¹ is hydrogen, halogen, hydroxy, alkyl, alkoxy, cyano or azido; preferably wherein

R¹ is hydrogen, fluorine, hydroxy, C₁₋₄-alkyl, C₁₋₄-alkoxy, cyano or azido;

R² is hydrogen or hydroxy; or

R² and R³ represent fluorine;

X is O or CH₂, preferably wherein

X is CH₂;

a, b, c, d denoting asymmetric carbon atoms each of which is substituted with 4 different substituents; and

B is a pyrimidine base B4 which is connected through the 1-nitrogen of formula

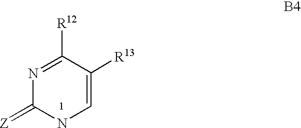

wherein

Z is O;

R¹² is NR⁷R⁸;

R¹³ is hydrogen, alkyl or halogen, preferably wherein

R¹³ is hydrogen, C₁₋₄-alkyl or fluorine;

R⁷ and R⁸ are independently of each other hydrogen or alkyl, preferably wherein

R⁷ and R⁸ are independently of each other hydrogen or C₁₋₄-alkyl; with the proviso that R¹² is not N(CH₃)₂ and R¹³ is not chlorine or bromine, preferably with the proviso that R¹² is not N(CH₃)₂;

for the treatment of diseases mediated by the Hepatitis C Virus (HIV) or for the preparation of a medicament for such treatment.

A further preferred embodiment of the invention is the use of compounds of formula I wherein

B is a pyrimidine base B5 which is connected through the 1-nitrogen of formula

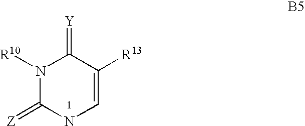

wherein

Y is O, S or NR¹¹;

Z is O or S;

R¹⁰ is hydrogen, alkyl or aryl;

R¹³ is hydrogen, alkyl, hydroxyalkyl, alkoxyalkyl, haloalkyl, cycloalkyl or halogen; with the proviso that R¹⁰ is not methyl or hydroxyethyl;

for the treatment of diseases mediated by the Hepatitis C Virus (HIV) or for the reparation of a medicament for such treatment.

More preferred embodiments for the use of compound of formula I for the treatment of diseases mediated by the Hepatitis C Virus or for the preparation of a medicament for such treatment are set out in table 1 (see below):

TABLE 1

| Example | STRUCTURE | Name |
|---|---|---|
| 1 | 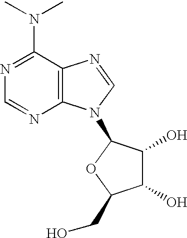 | 6-Dimethylamino-9-(β-D- ribofuranosyl)purine |
| 2 | 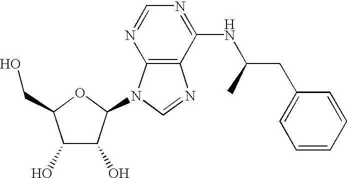 | 6-[1(S)-Methyl-2- phenylethylamino]-9-(β-D- ribofuranosyl)purine |
| 3 | 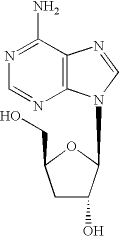 | 3′-Deoxyadenosine |
| 4 | 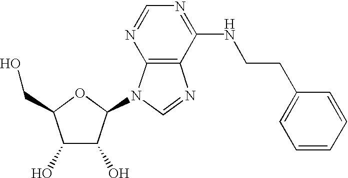 | 6-(Phenylethylamino)-9-(β-D- ribofuranosyl)purine |
| 5 | 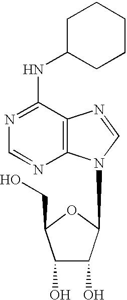 | 6-(Cyclohexylamino)-9-(β-D- ribofuranosyl)purine |
| 6 | 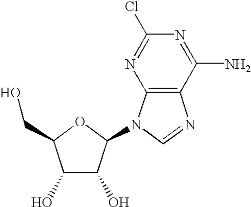 | 2-Chloroadenosine |
| 7 | 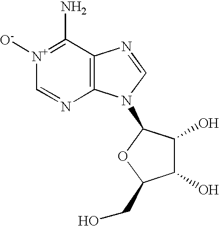 | Adenosine-1-oxide |
| 8 | 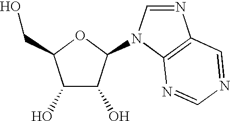 | 9-(β-D-Ribofuranosyl)purine |
| 9 | 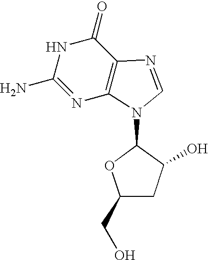 | 3′-Deoxyguanosine |
| 10 | 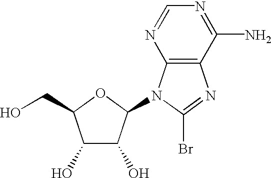 | 8-Bromoadenosine |
| 11 | 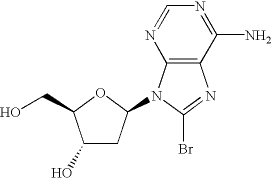 | 8-Bromo-2′-deoxyadenosine |
| 12 | 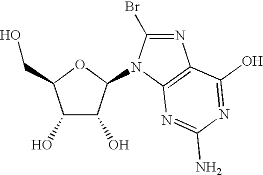 | 8-Bromoguanosine |
| 13 | 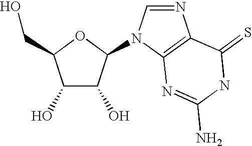 | 6-Thioguanosine |
| 14 | 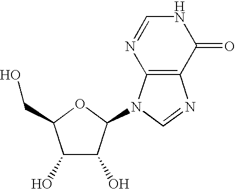 | Inosine |
| 15 | 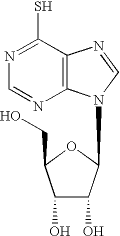 | 6-Thioinosine |
| 16 | 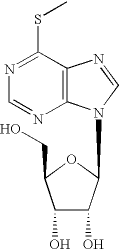 | 6-Methylthio-9-(β-D- ribofuranosyl)purine |
| 17 | 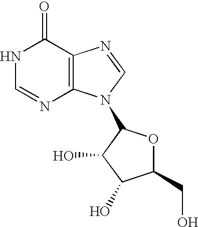 | L-Inosine |
| 18 | 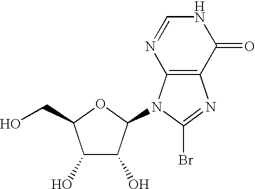 | 8-Bromoinosine |
| 19 | 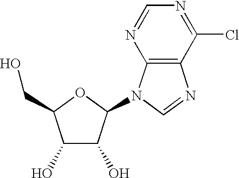 | 6-Chloro-9-(β-D- ribofuranosyl)purine |
| 20 | 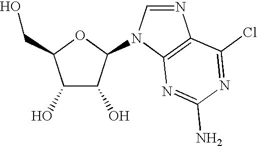 | 2-Amino-6-chloro-9-(β-D- ribofuranosyl)purine |
| 21 | 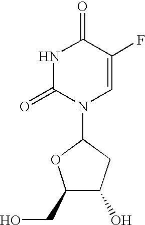 | 2′-Deoxy-5-fluorouridine |
| 22 | 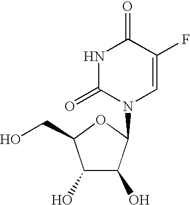 | 1-(β-D-Arabinofuranosyl)-5- fluorouracil |
| 23 | 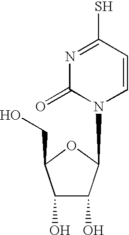 | 4-Thiouridine |
| 24 | 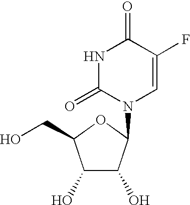 | 5-Fluorouridine |
| 25 | 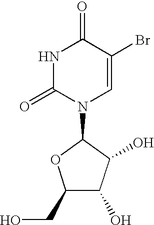 | 5-Bromouridine |
| 26 | 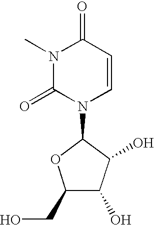 | 3-Methyluridine |
| 27 | 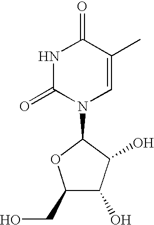 | 5-Methyluridine |
| 28 | 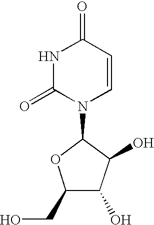 | 1-(β-D-Arabinofuranosyl)uracil |
| 29 | 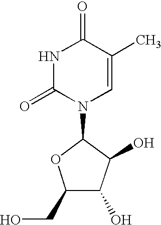 | 1-(β-D-Arabinofuranosyl)-5- methyluracil |
| 30 | 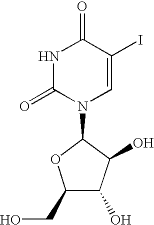 | 1-(β-D-Arabinofuranosyl)-5- iodouracil |
| 31 | 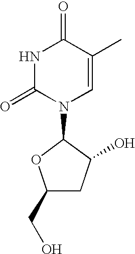 | 3′-Deoxy-5-methyluridine |
| 32 | 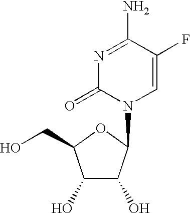 | 5-Fluorocytidine |
| 33 | 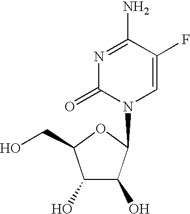 | 1-(β-D-Arabinofuranosyl)-5- fluorocytosine |
| 34 | 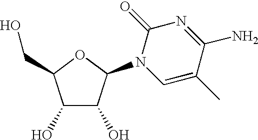 | 5-Methylcytidine |
| 35 | 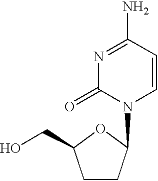 | 2′,3′-Dideoxycytidine |
| 36 | 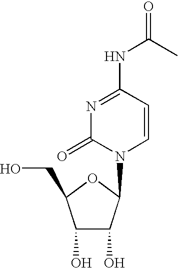 | N4-Acetylcytidine |
| 37 | 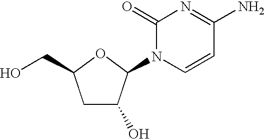 | 3′-Deoxycytidine |
| 38 | 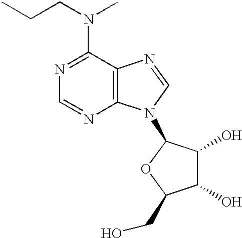 | 6-(N-Methylpropylamino)-9-(β- D-ribofuranosyl)purine |
| 39 | 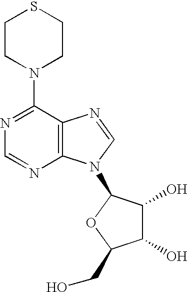 | 9-(β-D-Ribofuranosyl)-6-(4- thiomorpholinyl)purine |
| 40 | 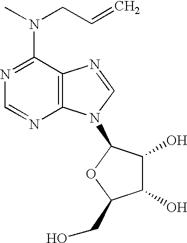 | 6-(N-Methyl-2- propenylamino)-9-(β-D- ribofuranosyl)purine |
| 41 | 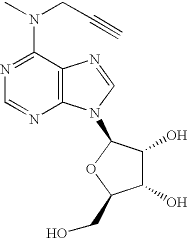 | 6-(N-Methyl-2-propynylamino)- 9-(β-D-ribofuranosyl)purine |
| 42 | 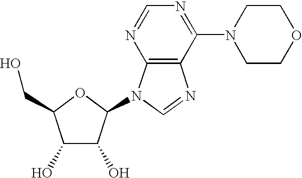 | 6-(4-Morpholinyl)-9-(β-D- ribofuranosyl)purine |
| 43 | 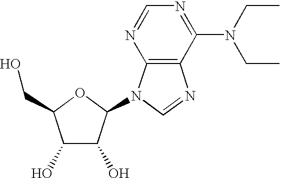 | 6-Diethylamino-9-(β-D- ribofuranosyl)purine |
| 44 | 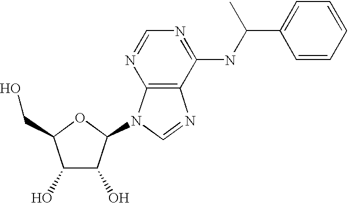 | 6-(1(R,S)-Phenylethylamino)-9- (β-D-ribofuranosyl)purine |
| 45 | 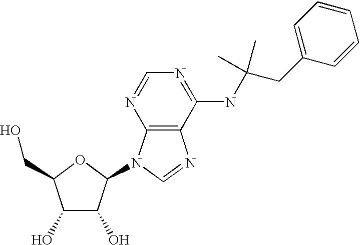 | 6-(1-Benzyl-1- methylethylamino)-9-(β-D- ribofuranosyl)purine |
| 46 | 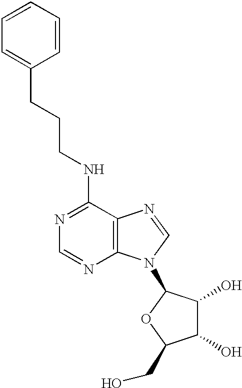 | 6-(3-Phenylpropylamino)-9-(β- D-ribofuranosyl)purine |
| 47 | 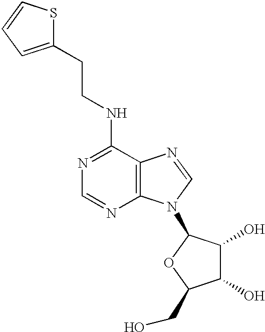 | 9-(β-D-Ribofuranosyl)-6-[2-(2- thienyl)ethylamino]purine |
| 48 | 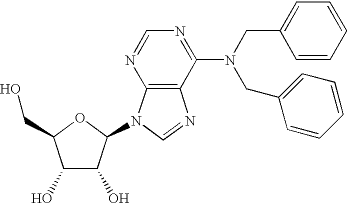 | 6-Dibenzylamino-9-(β-D- ribofuranosyl)purine |
| 49 | 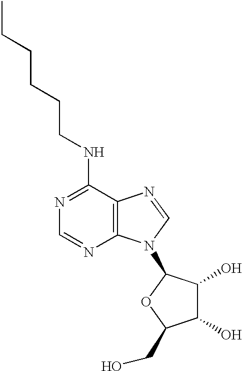 | 6-Hexylamino-9-(β-D- ribofuranosyl)purine |
| 50 | 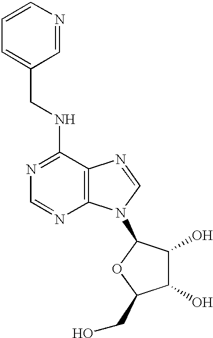 | 6-(3-Pyridylmethylamino)-9-(β- D-ribofuranosyl)purine |
| 51 | 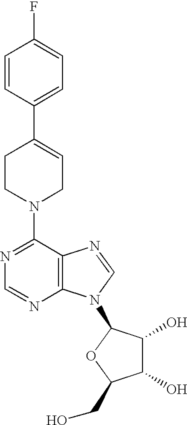 | 6-[4-(4-Fluorophenyl)-1,2,5,6- tetrahydropyridyl]-9-(β-D- ribofuranosyl)purine |
| 52 | 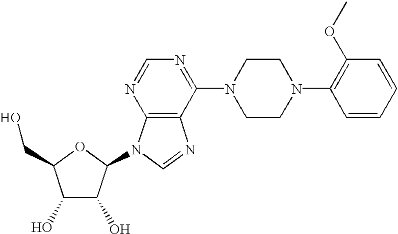 | 6-[4-(2- Methoxyphenyl)piperazinyl]-9- (β-D-ribofuranosyl)purine |
| 53 | 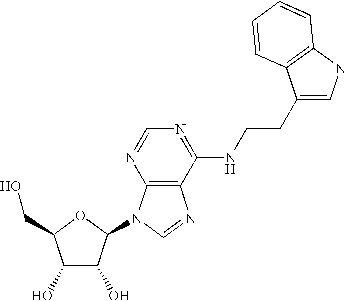 | 6-[2-(3-Indolyl)ethylamino]-9- (β-D-ribofuranosyl)purine |
| 54 | 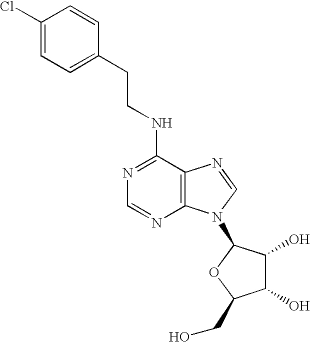 | 6-[2-(4- Chlorophenyl)ethylamino)]-9-(β- D-ribofuranosyl)purine |
| 55 | 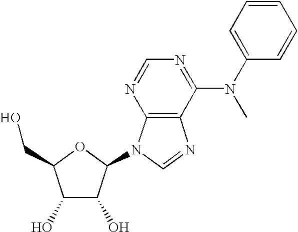 | 6-(N-Methylphenylamino)-9-(β- D-ribofuranosyl)purine |
| 56 | 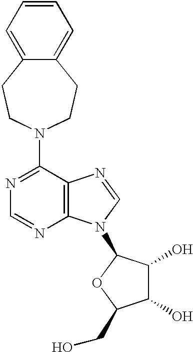 | 9-(β-D-Ribofuranosyl)-6- (1,2,4,5-tetrahydro-3H- benzazepin-3-yl)purine |
| 57 | 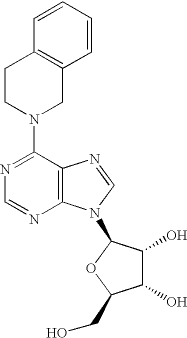 | 9-(β-D-Ribofuranosyl)-6- (1,2,3,4-tetrahydro-2- isoquinolyl)purine |
| 58 | 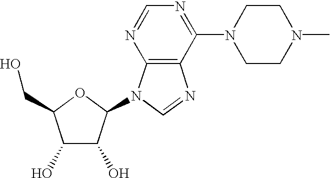 | 6-(4-Methylpiperazinyl)-9-(β-D- ribofuranosyl)purine |
| 59 | 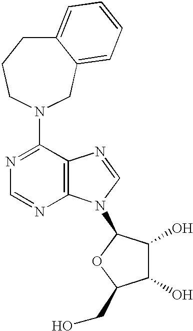 | 9-(β-D-Ribofuranosyl)-6- (1,3,4,5-tetrahydro-2H- benzazepin-2-yl)purine |
| 60 | 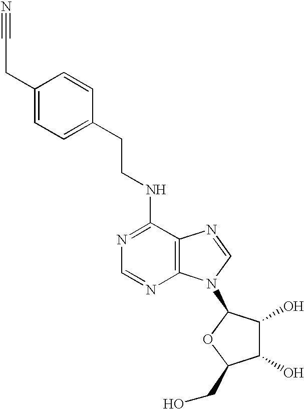 | 6-[2-(4- Cyanomethylphenyl)ethylamino]- 9-(β-D-ribofuranosyl)purine |
| 61 | 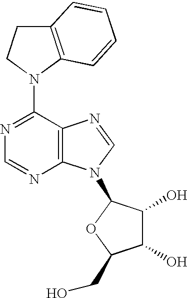 | 6-(2,3-Dihydro-1-indolyl)-9-(β- D-ribofuranosyl)purine) |
| 62 | 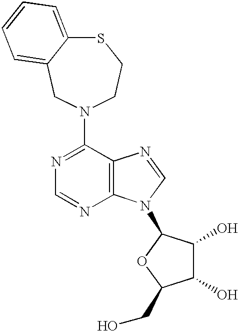 | 9-(β-D-Ribofuranosyl)-6- (2,3,4,5-tetrahydro-1,4- benzothiazepin-4-yl)purine |
| 63 | 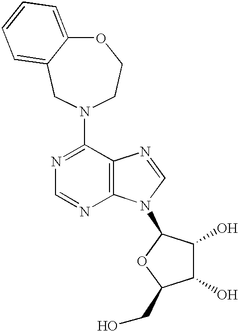 | 9-(β-D-Ribofuranosyl)-6- (2,3,4,5-tetrahydro-1,4- benzoxazepin-4-yl)purine |
| 64 | 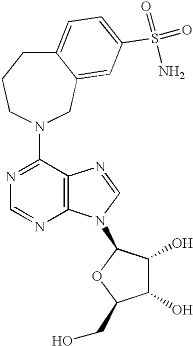 | 6-(8-Aminosulphonyl-2,3,4,5- tetrahydro-1H-2-benzazepin-2- yl)-9-(β-D-ribofuranosyl)purine |
| 65 | 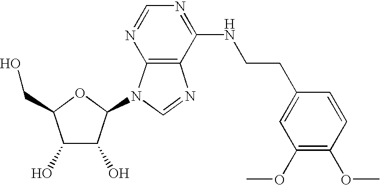 | 6-[2-(3,4- Dimethoxyphenyl)ethylamino)-9- (β-D-ribofuranosyl)purine |
| 66 | 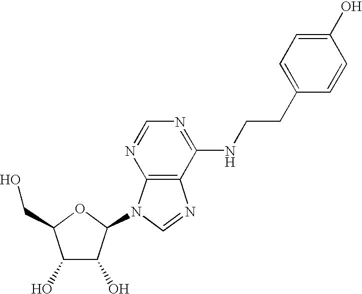 | 6-[-2-(4- Hydroxyphenyl)ethylamino]-9- (β-D-ribofuranosyl)purine |
| 67 | 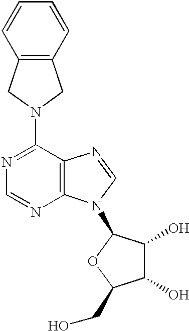 | 6-(2-Isoindolinyl)-9-(β-D- ribofuranosyl)purine |
| 68 | 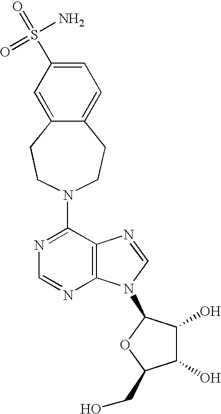 | 6-(7-Aminosulphonyl-2,3,4,5- tetrahydro-1H-benzazepin-3-yl)- 9-(β-D-ribofuranosyl)purine |
| 69 | 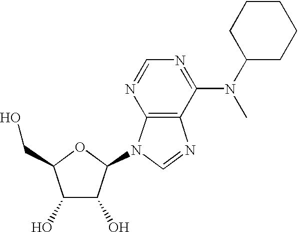 | 6-(N-Cyclohexylmethylamino)-9- (β-D-ribofuranosyl)purine |
| 70 | 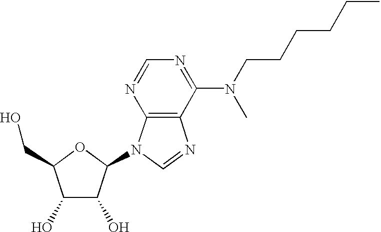 | 6-(N-Hexylmethylamino)-9-(β- D-ribofuranosyl)purine |
| 71 | 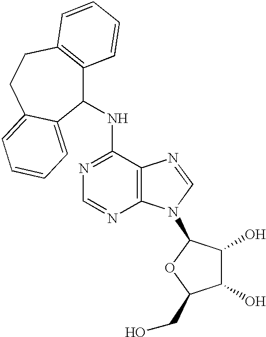 | 6-(10,11-Dihydro-5H- dibenzo[a,d]cyclohepten-5- ylamino)-9-(β-D- ribofuranosyl)purine |
| 72 | 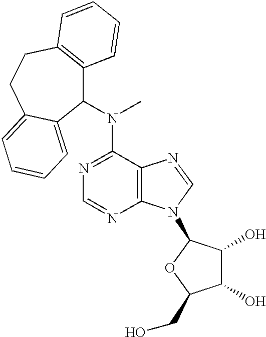 | 6-[N-(10,11-Dihydro-5H- dibenzo[a,d]cyclohepten-5- yl)methylamino]-9-(β-D- ribofuranosyl)purine |
| 73 | 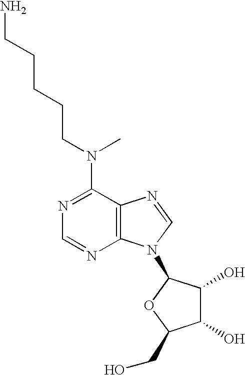 | 6-[N-(5- Aminopentyl)methylamino]-9- (β-D-ribofuranosyl)purine |
| 74 | 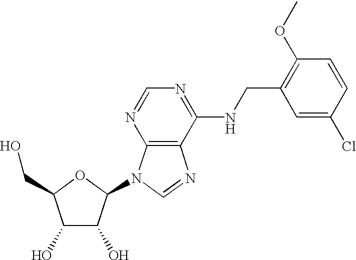 | 6-[(5-Chloro-2- methoxyphenyl)methylamino]-9- (β-D-ribofuranosyl)purine |
| 75 | 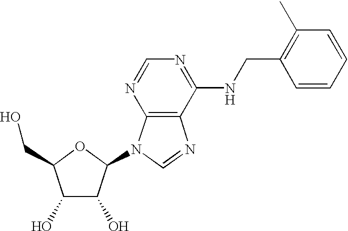 | 6-[(2- Methylphenyl)methylamino]-9- (β-D-ribofuranosyl)purine |
| 76 | 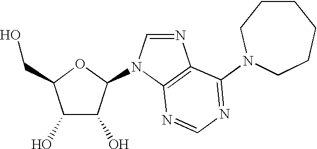 | 6-(Hexamethyleneimino)-9-(β-D- ribofuranosyl)purine |
| 77 | 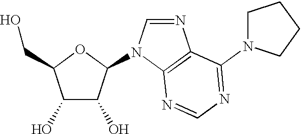 | 6-(1-Pyrrolidinyl)-9-(β-D- ribofuranosyl)purine |
| 78 | 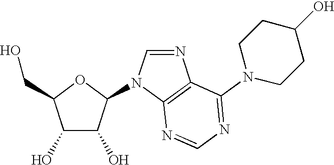 | 6-(4-Hydroxypiperidin-1-yl)-9- (β-D-ribofuranosyl)purine |
| 79 | 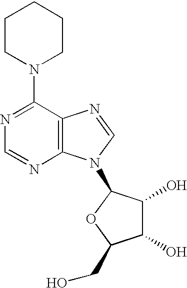 | 6-(1-Piperidinyl)-9-(β-D- ribofuranosyl)purine |
| 80 | 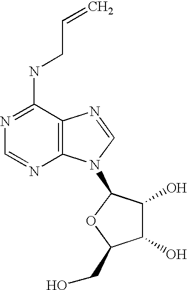 | 6-(2-Propenyl)amino-9-(β-D- ribofuranosyl)purine |
| 81 | 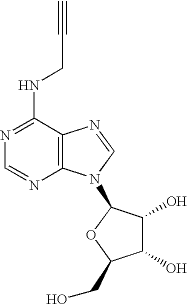 | 6-(2-Propynyl)amino-9-(β-D- ribofuranosyl)purine |
| 82 | 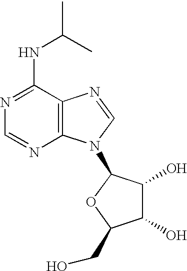 | 6-(1-Methyl)ethylamino-9-(β-D- ribofuranosyl)purine |
| 83 | 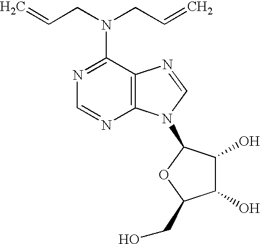 | 6-bis-(2-Propenyl)amino-9-(β-D- ribofuranosyl)purine |
| 84 | 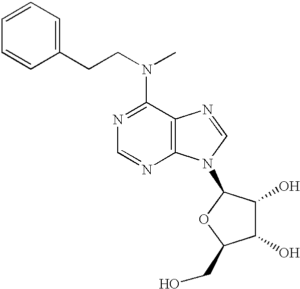 | 6-(2-Phenylethyl)methylamino-9- (β-D-ribofuranosyl)purine |
| 85 | 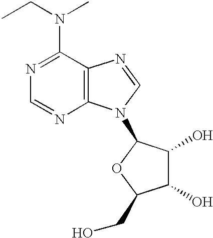 | 6-Ethylmethylamino-9-(β-D- ribofuranosyl)purine |
| 86 | 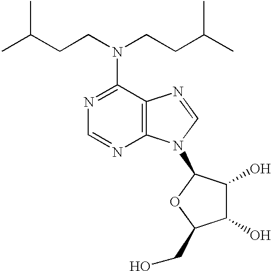 | 6-bis-[(3-Methyl)butylamino]-9- (β-D-ribofuranosyl)purine |
| 87 | 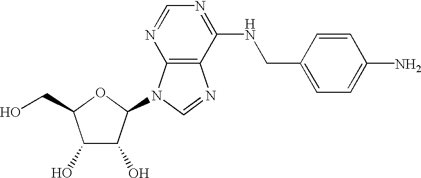 | 6-(4-Aminophenyl)methylamino- 9-(β-D-ribofuranosyl)purine |
| 88 | 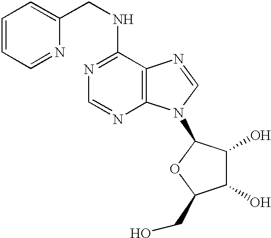 | 6-(2-Pyridylmethyl)amino-9-(β- D-ribofuranosyl)purine |
| 89 | 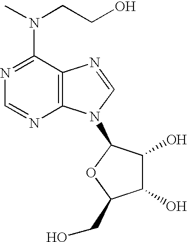 | 6-(2-Hydroxyethyl)methylamino- 9-(β-D-ribofuranosyl)purine |
| 90 | 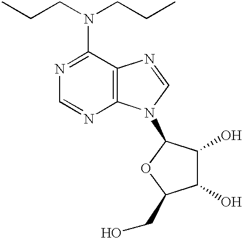 | 6-Dipropylamino-9-(β-D- ribofuranosyl)purine |
| 91 | 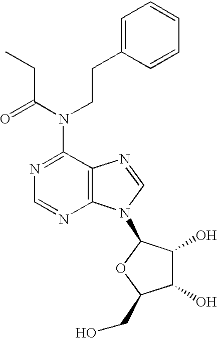 | 6-[2-Phenyl-(N- propionyl)ethylamino]-9-(β-D- ribofuranosyl)purine |
| 92 | 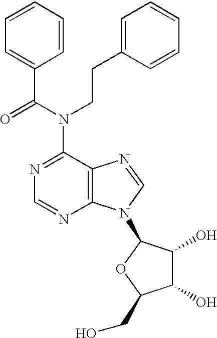 | 6-(N-Benzoyl-2- phenylethylamino)-9-(β-D- ribofuranosyl)purine |
| 93 | 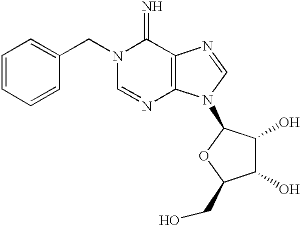 | 1-Benzyl-6-imino-9-(β-D- ribofuranosyl)purine |
| 94 | 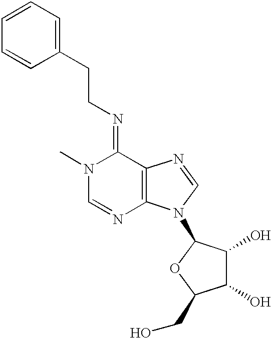 | 1-Methyl-6-(2- phenylethylimino)-9-(β-D- ribofuranosyl)purine |
| 95 | 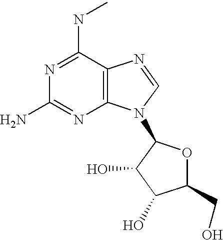 | 2-Amino-6-methylamino-9-(β-L- ribofuranosyl)purine |
| 96 | 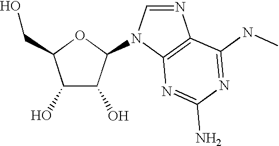 | 2-Amino-6-methylamino-9-(β-D- ribofuranosyl)purine |
| 97 | 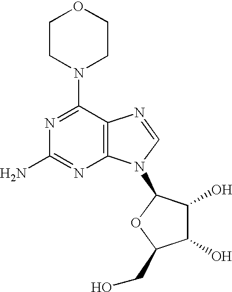 | 2-Amino-6-(4-morpholinyl)-9- (β-D-ribofuranosyl)purine |
| 98 | 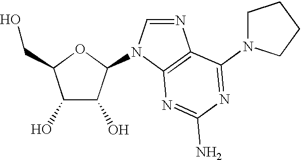 | 2-Amino-6-(1-pyrrolidinyl)-9-(β- D-ribofuranosyl)purine |
| 99 | 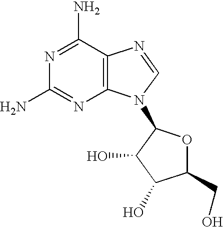 | 2,6-Diamino-9-(β-L- ribofuranosyl)purine |
| 100 | 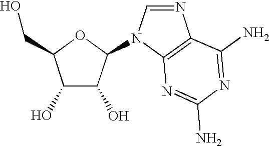 | 2,6-Diamino-9-(β-D- ribofuranosyl)purine |
| 101 | 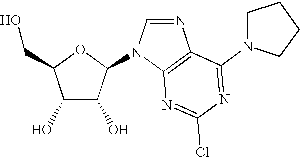 | 2-Chloro-6-(1-pyrrolidinyl)-9-(β- D-ribofuranosyl)purine |
| 102 | 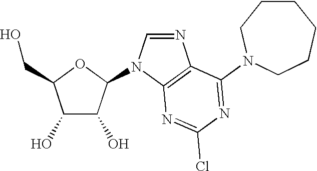 | 2-Chloro-6-(1- hexamethyleneimino)-9-(β-D- ribofuranosyl)purine |
| 103 | 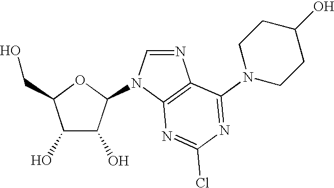 | 2-Chloro-6-(4-hydroxy-1- piperidinyl)-9-(β-D- ribofuranosyl)purine |
| 104 | 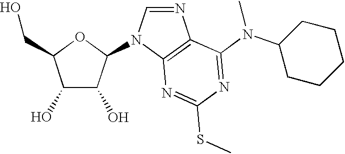 | 6-[(N-Cyclohexyl)methylamino]- 2-methylthio-9-(β-D- ribofuranosyl)purine |
| 105 | 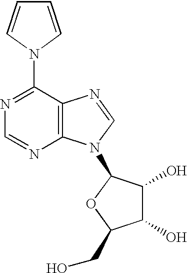 | 6-(1-Pyrrolyl)-9-(β-D- ribofuranosyl)purine |
| 106 | 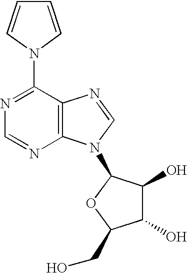 | 6-(1-Pyrrolyl)-9-(β-D- arabinofuranosyl)purine |
| 107 | 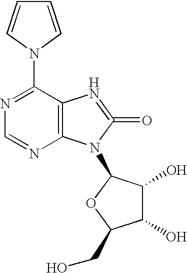 | 6-(1-Pyrrolyl)-9-(β-D- ribofuranosyl)purin-8-(7H)-one |
| 108 | 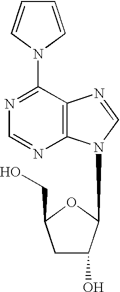 | 9-(3-Deoxy-β-D-ribofuranosyl)- 6-(1-pyrrolyl) purine |
| 109 | 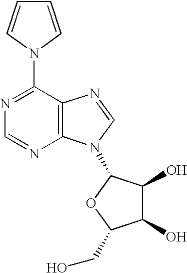 | 6-(1-Pyrrolyl)-9-(β-L- ribofuranosyl)purine |
| 110 | 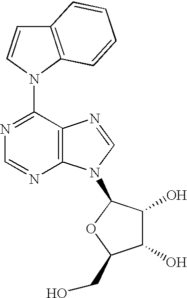 | 6-(1-Indolyl)-9-(β-D- ribofuranosyl)purine |
| 111 | 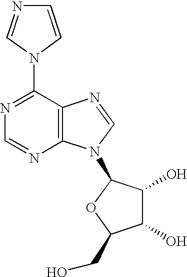 | 6-(1-Imidazolyl)-9-(β-D- ribofuranosyl)purine |
| 112 | 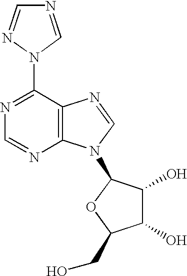 | 9-(β-D-Ribofuranosyl)-6-(1,2,4- triazol-1-yl)purine |
| 113 | 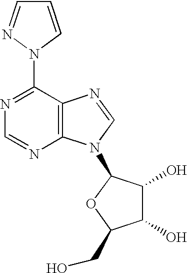 | 6-(1-Pyrazolyl)-9-(β-D- ribofuranosyl)purine |
| 114 | 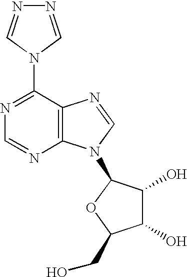 | 9-(β-D-ribofuranosyl) 6-(1,2,4- triazol-4-yl)purine |
| 115 | 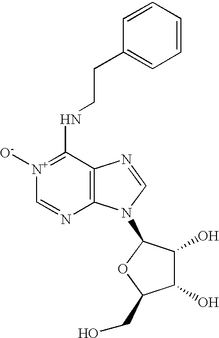 | 6-(2-Phenylethylamino)-9-(β-D- ribofuranosyl)purine-1-oxide |
| 116 | 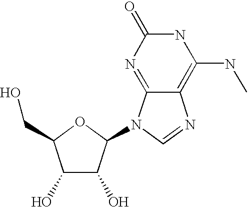 | 6-Methylamino-9-(β-D- ribofuranosyl)purin-2(1H)-one |
| 117 | 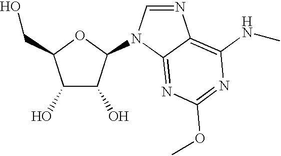 | 2-Methoxy-6-methylamino-9-(β- D-ribofuranosyl)purine |
| 118 | 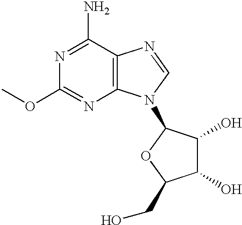 | 2-Methoxyadenosine |
| 119 | 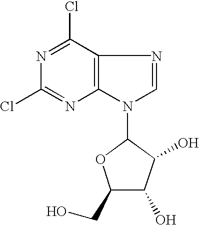 | 2,6-Dichloro-9-(β-D- ribofuranosyl)purine |
| 120 | 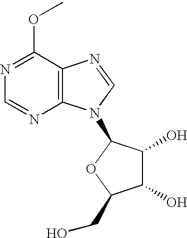 | 6-Methoxy-9-(β-D- ribofuranosyl)purine |
| 121 | 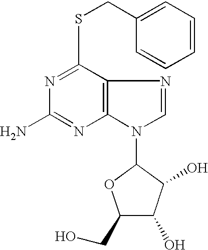 | 2-Amino-6-benzylthio-9-(β-D- ribofuranosyl)purine |
| 122 | 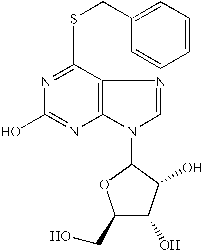 | 6-Benzylthio-2-hydroxy-9-(β-D- ribofuranosyl)purine |
| 123 | 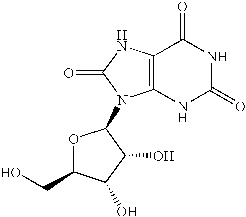 | 9-(β-D-Ribofuranosyl)purine- 2,6,8(1H,3H,7H)-trione |
| 124 | 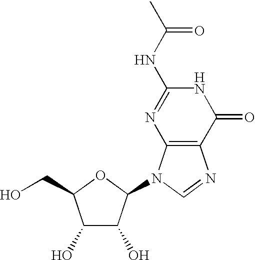 | 2-(Acetylamino)inosine |
| 125 | 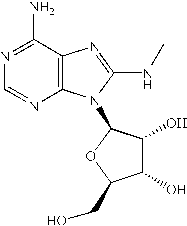 | 8-(Methylamino)adenosine |
| 126 | 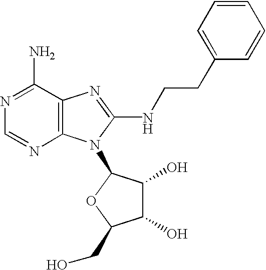 | 8-(2-Phenylethylamino) adenosine |
| 127 | 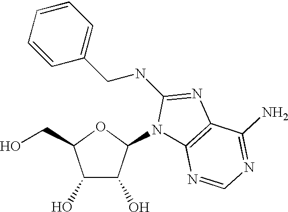 | 8-Benzylaminoadenosine |
| 128 | 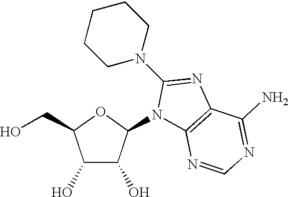 | 8-(1-Piperidinyl)adenosine |
| 129 | 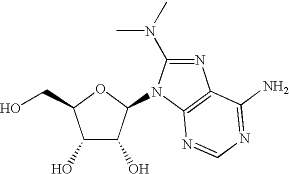 | 8-(Dimethylamino)adenosine |
| 130 | 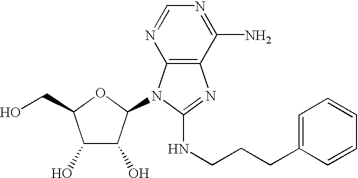 | 8-(3-Phenylpropylamino) adenosine |
| 131 | 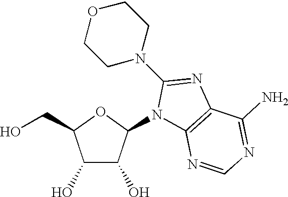 | 8-(4-Morpholinyl)adenosine |
| 132 | 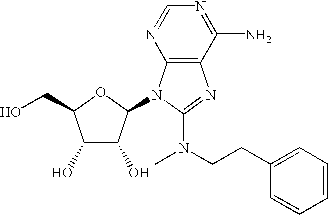 | 8-(N-Methyl-2- phenylethylamino)adenosine |
| 133 | 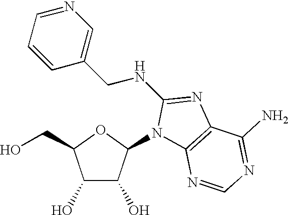 | 8-(3-Pyridylmethylamino) adenosine |
| 134 | 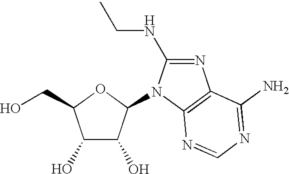 | 8-(Ethylamino)adenosine |
| 135 | 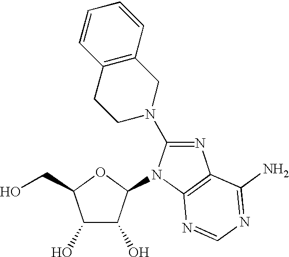 | 8-(1,2,3,4-Tetrahydro-2- isoquinolyl)adenosine |
| 136 | 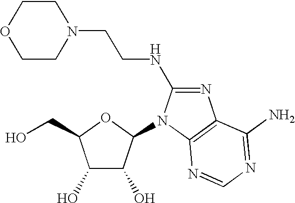 | 8-[2-(4-Morpholinyl)ethylamimo]adenosine |
| 137 | 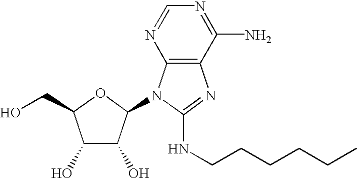 | 8-(Hexylamino)adenosine |
| 138 | 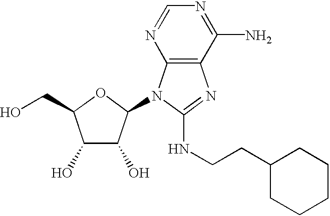 | 8-(2-Cyclohexylethylamino) adenosine |
| 139 | 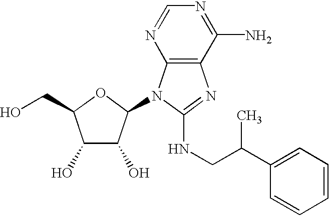 | 8-(2(R,S)-Phenylpropylamino) adenosine |
| 140 | 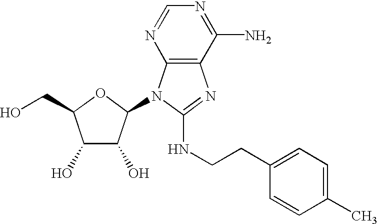 | 8-[2-(4-Methylphenyl) ethylamino]adenosine |
| 141 | 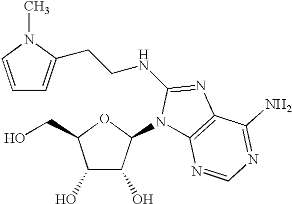 | 8-[2-(1-Methyl-2-pyrrolyl) ethylamino]adenosine |
| 142 | 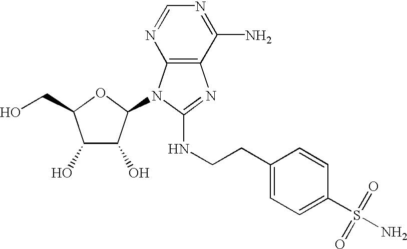 | 8-[2-(4-Aminosulphonylphenyl) ethylamino]adenosine |
| 143 | 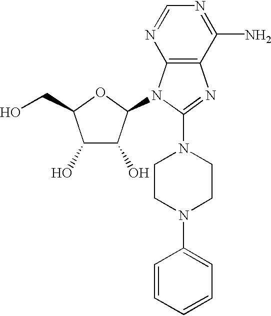 | 8-(4-Phenyl-1-piperazinyl) adenosine |
| 144 | 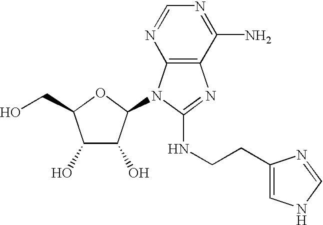 | 8-(2-(4-Imidazolyl)adenosine |
| 145 | 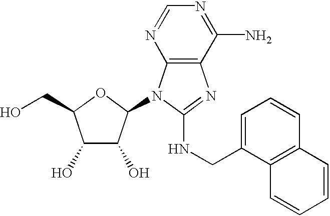 | 8-(1- Naphthylmethylamino)adenosine |
| 146 | 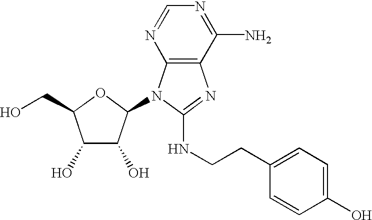 | 8-[2-(4- Hydroxyphenyl)ethylamino]adenosine |
| 147 | 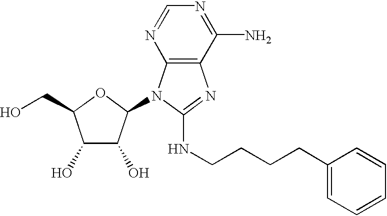 | 8-(4-Phenylbutylamino) adenosine |
| 148 | 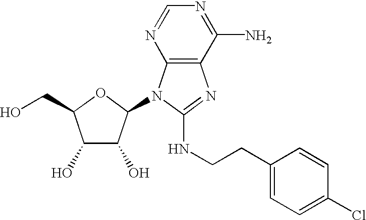 | 8-[2-(4- Chlorophenyl)ethylamino]adenosine |
| 149 | 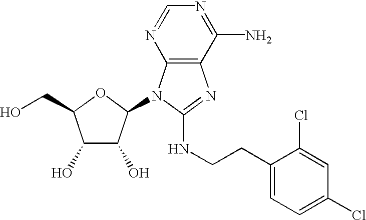 | 8-[2-(2,4- Dichlorophenyl)ethylamino]adenosine |
| 150 | 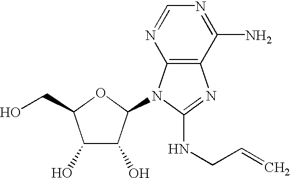 | 8-(2-Propenylamino)adenosine |
| 151 | 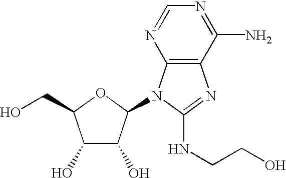 | 8-(2-Hydroxyethylamino) adenosine |
| 152 | 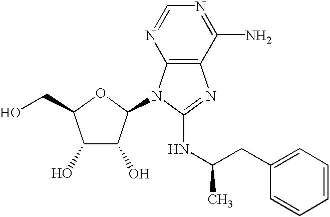 | 8-(1(R)-Methyl-2- phenylethylamino)adenosine |
| 153 | 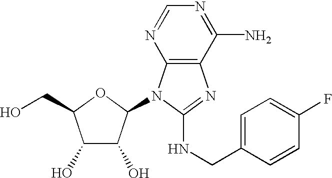 | 8-(4- Fluorobenzylamino)adenosine |
| 154 | 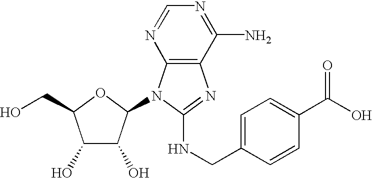 | 8-[(4-Hydroxycarbonyl)- benzylamino]adenosine |
| 155 | 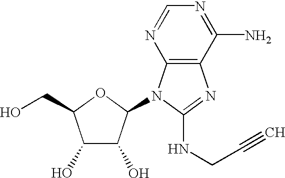 | 8-(2-Propynylamino)adenosine |
| 156 | 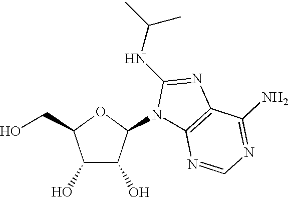 | 8-(1-Methylethylamino)adenosine |
| 157 | 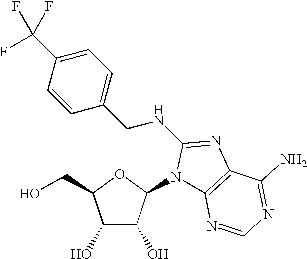 | 8-[(4- Trifluoromethyl)benzylamino]adenosine |
| 158 | 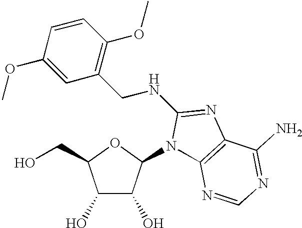 | 8-[(2,5-Dimethoxy)benzylamino]adenosine |
| 159 | 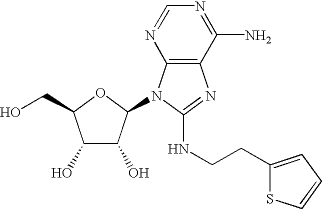 | 8-[2-(2-thienyl)ethylamino]adenosine |
| 160 | 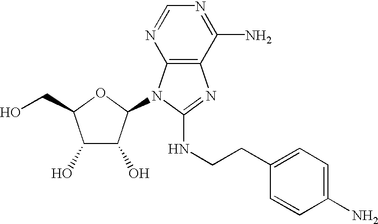 | 8-[2-(4- Aminophenyl)ethylamino]adenosine |
| 161 | 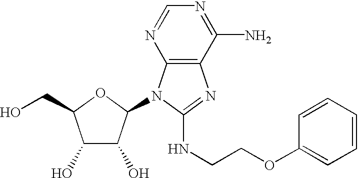 | 8-(2- Phenoxyethylamino)adenosine |
| 162 | 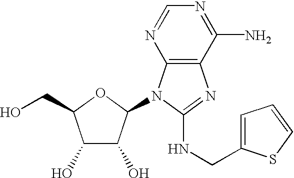 | 8-[(2-Thienyl)methylamino) adenosine |
| 163 | 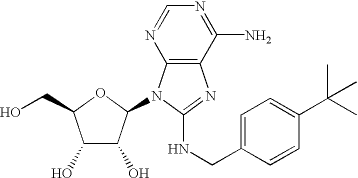 | 8-[(4-tert-Butyl)benzylamino]adenosine |
| 164 | 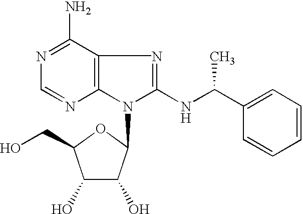 | 8-(1(R)-Phenylethylamino) adenosine |
| 165 | 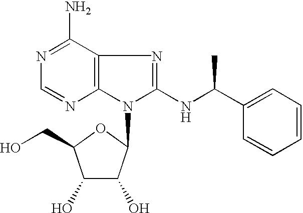 | 8-(1(S)-Phenylethylamino) adenosine |
| 166 | 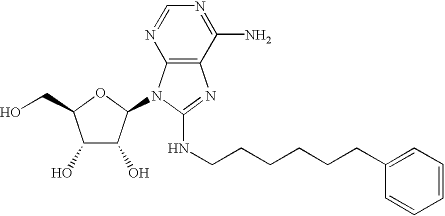 | 8-(6- Phenylhexylamino)adenosine |
| 167 | 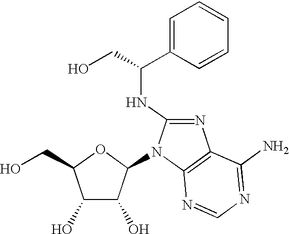 | 8-[2-Hydroxy-1(S)- phenyl)ethylamino]adenosine |
| 168 | 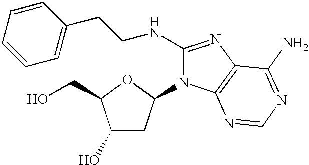 | 2′-Deoxy-8-(2- phenylethylamino)adenosine |
| 169 | 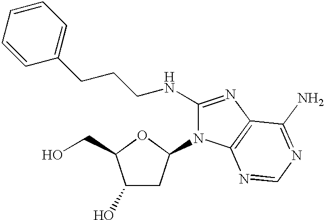 | 2′-Deoxy-8-(3- phenylpropylamino)adenosine |
| 170 | 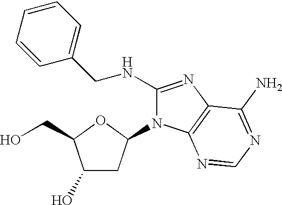 | 8-Benzylamino-2′- deoxyadenosine |
| 171 | 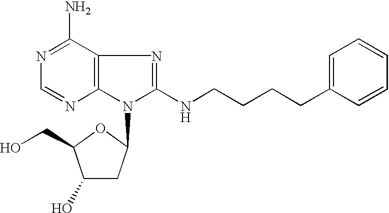 | 2′-Deoxy-8-(4- phenylbutylamino)adenosine |
| 172 | 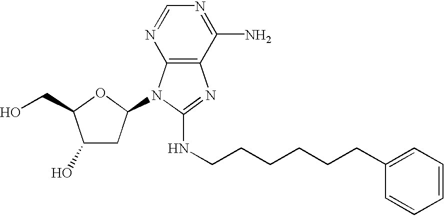 | 2′-Deoxy-8-(6- phenylhexylamino)adenosine |
| 173 | 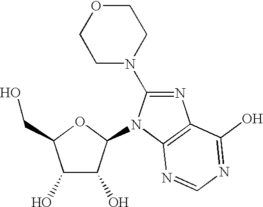 | 8-(4-Morpholinyl)inosine |
| 174 | 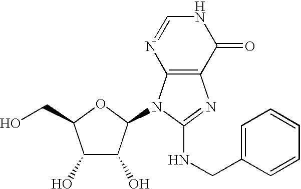 | 8-(Benzylamino)inosine |
| 175 | 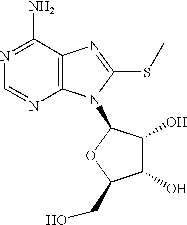 | 8-(Methylthio)adenosine |
| 176 | 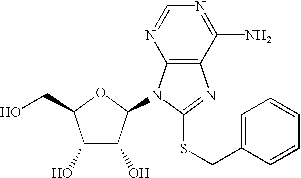 | 8-(Benzylthio)adenosine |
| 177 | 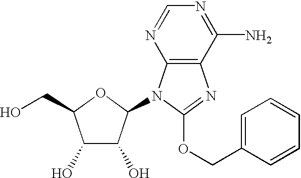 | 8-(Benzyloxy)adenosine |
| 178 | 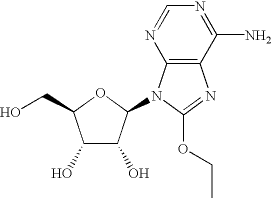 | 8-Ethoxyadenosine |
| 179 | 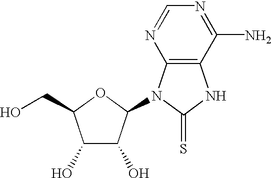 | 6-Amino-9-(β-D- ribofuranosyl)purine-8(7H)- thione |
| 180 | 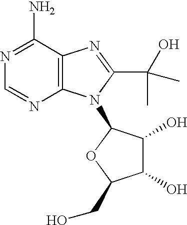 | 8-[(1-Hydroxy-1-methyl)ethyl]adenosine |
| 181 | 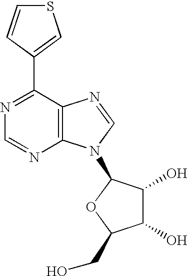 | 9-(β-D-Ribofuranosyl)-6-(3- thienyl)purine |
| 182 | 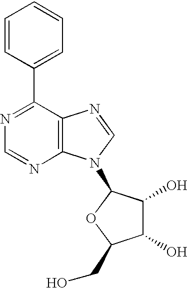 | 6-Phenyl-9-(β-D- ribofuranosyl)purine |
| 183 | 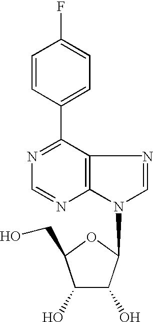 | 6-(4-Fluorophenyl)-9-(β-D- ribofuranosyl)purine |
| 184 | 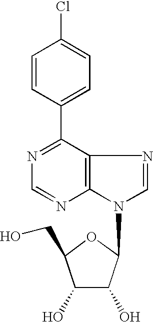 | 6-(4-Chlorophenyl)-9-(β-D- ribofuranosyl)purine |
| 185 | 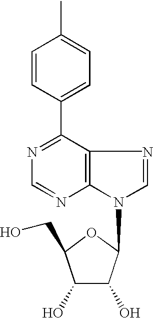 | 6-(4-Methylphenyl)-9-(β-D- ribofuranosyl)purine |
| 186 | 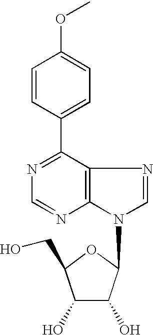 | 6-(4-Methoxyphenyl)-9-(β-D- ribofuranosyl)purine |
| 187 | 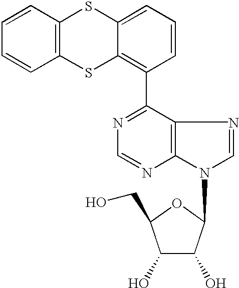 | 9-(β3-D-Ribofuranosyl)-6-(1- thianthrenyl)purine |
| 188 | 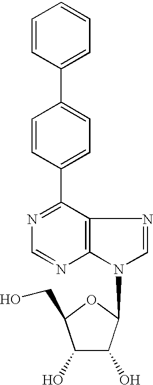 | 6-(4-Biphenylyl)-9-(β-D- ribofuranosyl)purine |
| 189 | 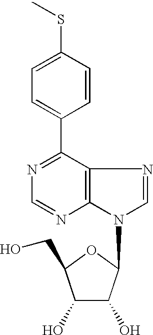 | 6-(4-Methylthiophenyl)-9-(β-D- ribofuranosyl)purine |
| 190 | 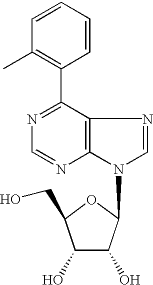 | 6-(2-Methylphenyl)-9-(β-D- ribofuranosyl)purine |
| 191 | 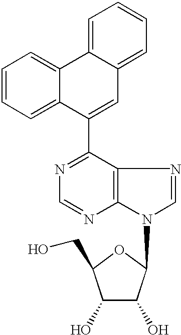 | 6-(9-Phenanthrenyl)-9-(β-D- ribofuranosyl)purine |
| 192 | 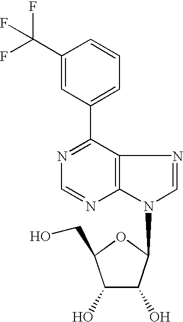 | 9-(β-D-Ribofuranosyl)-6-(3- trifluoromethylphenyl)purine |
| 193 | 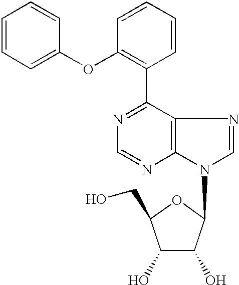 | 6-(2-Phenoxyphenyl)-9-(β-D- ribofuranosyl)purine |
| 194 | 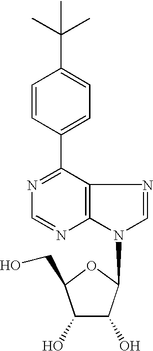 | 6-(4-tert-Butylphenyl)-9-(β-D- ribofuranosyl)purine |
| 195 | 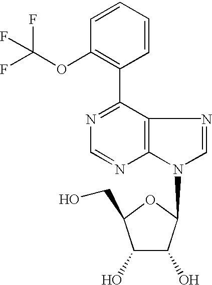 | 9-(β-D-Ribofuranosyl)-6-(2- trifluoromethoxyphenyl)purine |
| 196 | 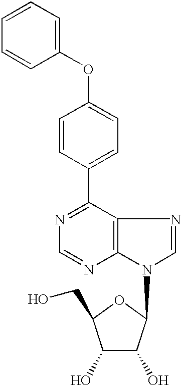 | 6-(4-Phenoxyphenyl)-9-(β-D- ribofuranosyl)purine |
| 197 | 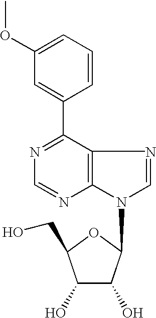 | 6-(3-Methoxyphenyl)-9-(β-D- ribofuranosyl)purine |
| 198 | 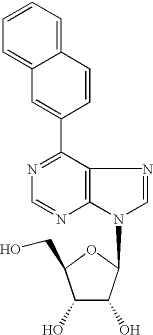 | 6-(2-Naphthyl)-9-(β-D- ribofuranosyl)purine |
| 199 | 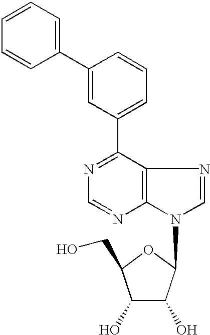 | 6-(3-Biphenylyl)-9-(β-D- ribofuranosyl)purine |
| 200 | 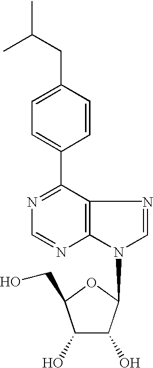 | 6-[4-(2-Methylpropyl)phenyl]-9- (β-D-ribofuranosyl)purine |
| 201 | 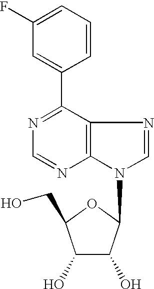 | 6-(3-Fluorophenyl)-9-(β-D- ribofuranosyl)purine |
| 202 | 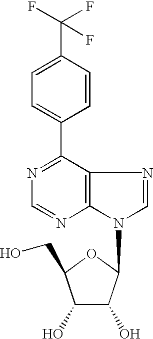 | 9-(β-D-Ribofuranosyl)-6-(4- trifluoromethylphenyl)purine |
| 203 | 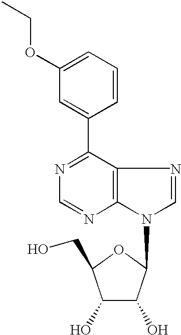 | 6-(3-Ethoxyphenyl)-9-(β-D- ribofuranosyl)purine |
| 204 | 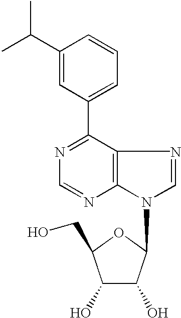 | 6-[3-(1-Methyl)ethylphenyl]-9- (β-D-ribofuranosyl)purine |
| 205 | 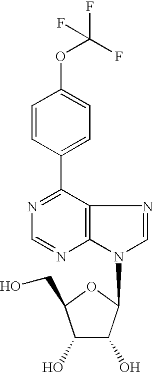 | 9-(β-D-Ribofuranosyl)-6-(4- trifluoromethoxyphenyl)purine |
| 206 | 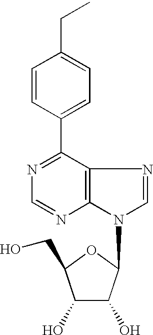 | 6-(4-Ethylphenyl)-9-(β-D- ribofuranosyl)purine |
| 207 | 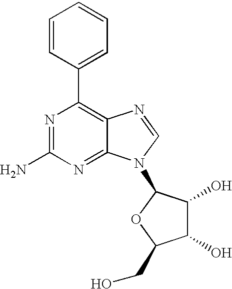 | 2-Amino-6-phenyl-9-(β-D- ribofuranosyl)purine |
| 208 | 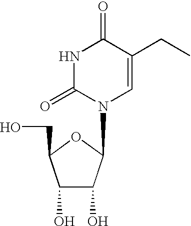 | 5-Ethyluridine |
| 209 | 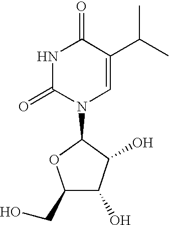 | 5-[(1-Methyl)ethyl]uridine |
| 210 | 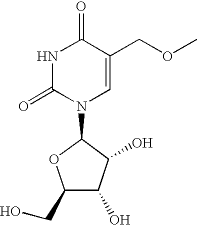 | 5-Methoxymethyluridine |
| 211 | 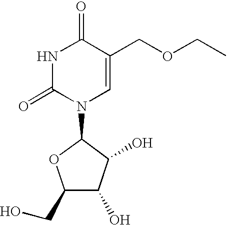 | 5-Ethoxymethyluridine |
| 212 | 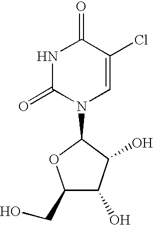 | 5-Chlorouridine |
| 213 | 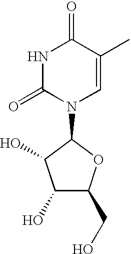 | 5-Methyl-1-(β-L- ribofuranosyl)uracil |
| 214 | 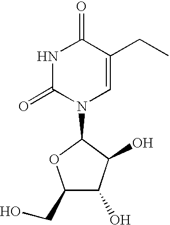 | 1-(β-D-Arabinofuranosyl)-5- ethyluracil |
| 215 | 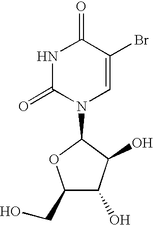 | 1-(β-D-Arabinofuranosyl)-5- bromouracil |
| 216 | 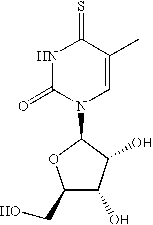 | 5-Methyl-4-thiouridine |
| 217 | 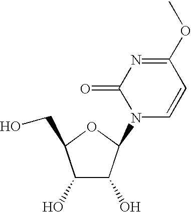 | 4-Methoxy-1-(β-D- ribofuranosyl)pyrimidin-2(1H)- one |
| 218 | 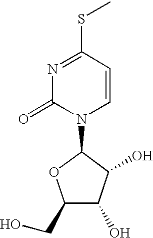 | 4-Methylthio-1-(β-D- ribofuranosyl)pyrimidin-2(1H)- one |
| 219 | 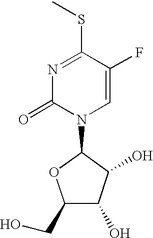 | 5-Fluoro-4-methylthio-1-(β-D- ribofuranosyl)pyrimidin-2(1H)- one |
| 220 | 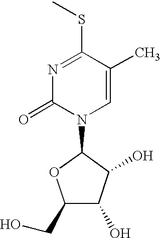 | 5-Methyl-4-methylthio-1-(β-D- ribofuranosyl)pyrimidin-2(1H)- one |
| 221 | 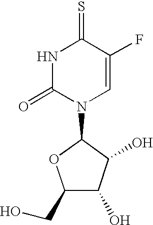 | 5-Fluoro-4-thiouridine |
| 222 | 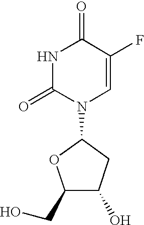 | 1-(2-Deoxy-α-D-erthyro- pentofuranosyl)-5-fluorouracil |
| 223 | 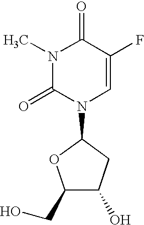 | 2′-Deoxy-5-fluoro-3- methyluridine |
| 224 | 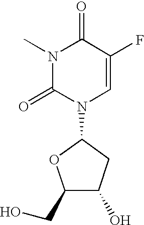 | 1-(α-D-Erthyro-2- deoxypentofuranosyl)-5-fluoro-3- methyluracil |
| 225 | 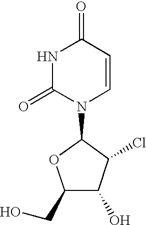 | 2′-Chloro-2′-deoxyuridine |
| 226 | 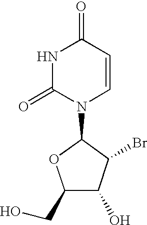 | 2′-Bromo-2′-deoxyuridine |
| 227 | 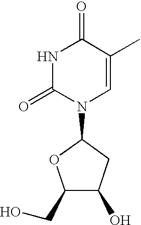 | 1-(2-Deoxy-β-D-lyxofuranosyl)- 5-methyluracil |
| 228 | 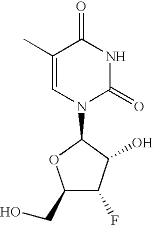 | 3′-Deoxy-3′-fluoro-5- methyluridine |
| 229 | 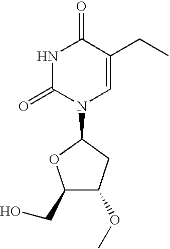 | 2′,3′-Dideoxy-5-ethyl-3′- methoxyuridine |
| 230 | 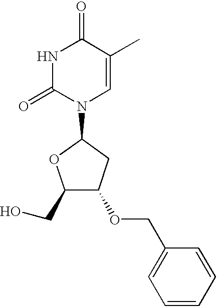 | 5′-Benzyloxy-2′,3′-dideoxy-5- methyluridine |
| 231 | 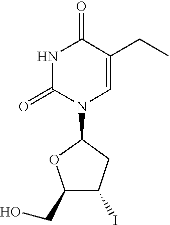 | 2′,3′-Dideoxy-5-ethyl-3′- iodouridine |
| 232 | 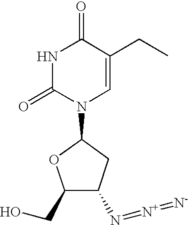 | 3′-Azido-2′,3′-dideoxy-5- ethyluridine |
| 233 | 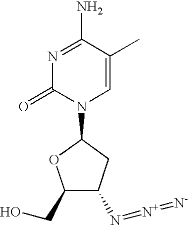 | 3′-Azido-2′,3′-dideoxy-5- methylcytidine |
| 234 | 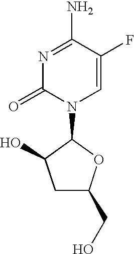 | 1-(3-Deoxy-β-L-threo-pentofuranosyl)- 5-fluorocytosine |
| 235 | 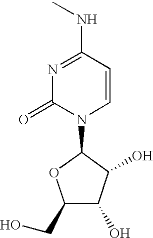 | 4-Methylamino-1-(β-D- ribofuranosyl)pyrimidin-2(1H)- one |
| 236 | 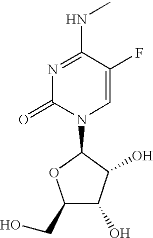 | 5-Fluoro-4-methylamino-1-(β-D- ribofuranosyl)pyrimidin-2(1H)- one |
| 237 | 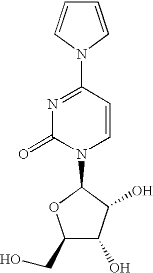 | 4-(1-Pyrrolyl)-1-(β-D- ribofuranosyl)pyrimidin-2(1H)- one |
| 238 | 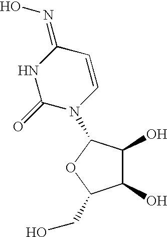 | 4-Oximino-1-(β-L- ribofuranosyl)pyrimidin-2(1H)- one |
| 239 | 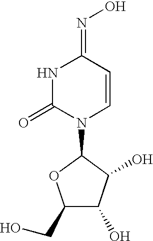 | 4-Oximino-1-(β-D- ribofuranosyl)pyrimidin-2(1H)- one |
| 240 | 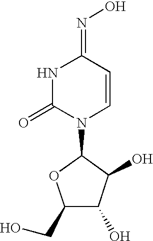 | 4-Oximino-1-(β-D- arabinofuranosyl)pyrimidin- 2(1H)-one |
| 241 | 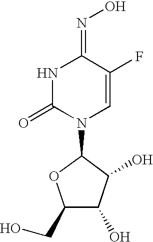 | 5-Fluoro-4-oximino-1-(β-D- ribofuranosyl)pyrimidin-2(1H)- one |
| 242 | 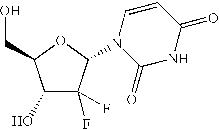 | 1-(2-Deoxy-2,2-difluoro-α-D- erythropentofuranosyl)uracil |
| 243 | 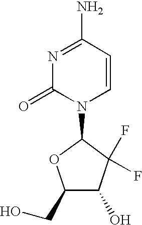 | 1-(2-Deoxy-2,2-difluoro-β-D- erythropentofuranosyl)cytosine |
| 244 | 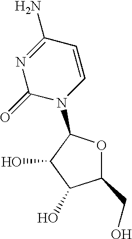 | L-Cytidine |
| 245 | 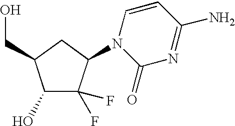 | 4-Amino-1-(2,2-difluoro-3- hydroxy-4-hydroxymethyl- cyclopentyl)-1H-pyrimidin-2-one |
| 246 | 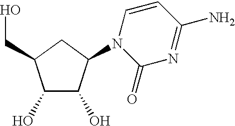 | 4-Amino-1(R)-(2(S),3(R)- dihydroxy-4(R)-hydroxymethyl- cyclopentyl)-1H-pyrimidin-2- one |
| 247 | 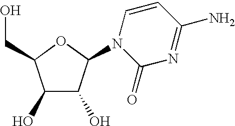 | 1-(β-D-Xylofuranosyl)cytosine |
| 248 | 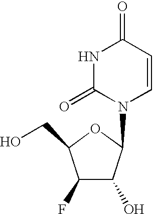 | 1-(3-Deoxy-3-fluoro-β-D- xylofuranosyl)uracil |
| 249 | 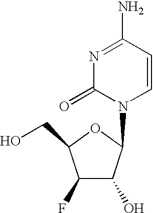 | 1-(3-Deoxy-3-fluoro-β-D- xylofuranosyl)cytosine |
| 250 | 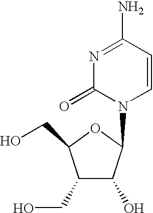 | 3′-Deoxy-3′- hydroxymethylcytidine |
| 251 | 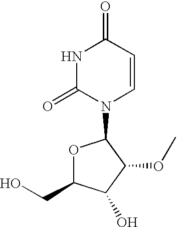 | 2′-Deoxy-2′-methoxyuridine |
| 252 | 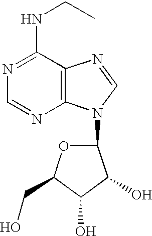 | 6-Ethylamino-9-(β-D- ribofuranosyl)purine |
| 253 | 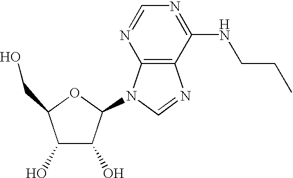 | 6-Propylamino-9-(β-D- ribofuranosyl)purine |

The compounds of formula I according to the present invention are prepared as follows:

The compounds of formula I may be prepared by various methods known in the art organic chemistry in general and nucleoside analogue synthesis in particular. The starting materials for the syntheses are either readily available from commercial sources or are known or may themselves be prepared by techniques known in the art. General reviews of the preparation of nucleoside analogues are included in the following:

A M Michelson “The Chemistry of Nucleosides and Nucleotides”, Academic Press, New York 1963.

L Goodman “Basic Principles in Nucleic Acid Chemistry” ed P O P Ts'O, Academic Press, New York 1974, Vol. 1, chapter 2.

“Synthetic Procedures in Nucleic acid Chemistry” ed W W Zorbach and R S Tipson, Wiley, New York, 1973, Vol. 1 and 2.

The synthesis of carbocylic nucleosides has been reviewed by: L Agrofoglio et al Tetrahedron, 1994, 50, 10611.

The strategies available for the synthesis of compounds of formula I include:

Condensation of a protected furanose, thiofuranose or cyclopentane derivative of formula II

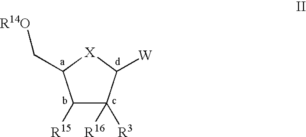

wherein

R³ is as defined above;

R¹⁴ is a hydroxy protecting group;

R¹⁵ is as defined for R¹ except that when R¹ is hydroxy R¹⁵ is a group OR¹⁷ wherein

R¹⁷ is a hydroxy protecting group;

R¹⁶ is as defined for R² except that when R² is hydroxy R¹⁶ is a group OR¹⁷ wherein

R¹⁷ is a hydroxy protecting group;

X is O, S or CH₂;

W is a leaving group such as acyloxy, aryloxy, alkylsulphonate, arylsulphonate, S-benzyl or halogen; and

a, b, c, d denoting asymmetric carbon atoms each of which is substituted with 4 different substituents;

with an appropriate purine of formula III

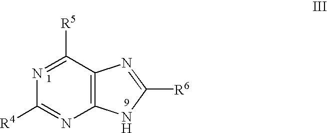

wherein R⁴, R⁵ and R⁶ are as defined in formula I;

or pyrimidine of formula IV

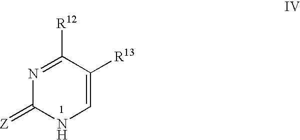

wherein Z, R¹² and R¹³ are as defined in formula I;

or a derivative of the purine or pyrimidine such as for example a heavy metal or silyl derivative.

The particular nature of the hydroxy protecting groups R¹⁴ or R¹⁷ is selected in accordance with conventional techniques. Examples for hydroxy protecting groups are acyl (e.g. acetyl), aroyl (e.g. benzoyl), ether (e.g. bis-acetonide), silylether (e.g. trimethylsilyl, tert-butyldimethylsilyl) or arylmethyl (e.g. benzyl, triphenylmethyl).

The condensation reaction maybe performed using standard methods including the use of a Lewis acid catalyst such as mercuric bromide or stannic chloride or trimethylsilyltrifluoromethane sulphonate in solvents such as acetonitrile, 1,2-dichloroethane, dichloromethane, chloroform or toluene at reduced, ambient or elevated temperature. Examples for the condensation reaction of a protected furanose or thiofuranose of formula II where X is O or S with an appropriate pyrimidine or purine derivative are as follows:

The reaction may be performed by the condensation of heavy metal derivatives of purines of formula III or pyrimidines of formula IV (e.g. chloromercuri derivatives) with a compound of formula II as described by J Davoll and B A Lowry J Am Chem Soc 1951, 73, 1650; J J Fox, N Yung, J Davoll and G B Brown J Am Chem Soc 1956, 78, 2117.

The reaction may also involve the condensation of alkoxy pyrimidines with compounds of formula II as described by K A Watanabe, D H Hollenberg and J J Fox Carbohydrates, Nucleosides and Nucleotides 1974, 1,1.

The reaction may be performed by the condensation of silyl derivatives of purines of formula III or pyrimidines of formula IV with compounds of formula II as described by U Niedballa and H Vorbruggen J Org Chem 1976, 41, 2084; U Niedballa and H Vorbruggen J Org Chem 1974, 39, 3672. A J Hubbard, A S Jones and R T Walker Nucleic Acids Res 1984, 12, 6827.

Fusion of per-acylated sugars with purines under vacuum in the presence of p-toluene sulphonic acid has been described by T Simadate, Y Ishudo and T Sato Chem Abs 1962, 56, 11 692 and W Pfleiderer, R K Robins Chem Ber 1965, 98, 1511.

Further coupling reactions have been described by K A Watanabe, D H Hollenberg and J J Fox Carbohydrates, Nucleosides and Nucleotides 1974, 1,1.

Examples for the condensation reaction of a protected cyclopentane derivative of formula II wherein X is CH₂with an appropriate purine derivative of formula III or pyrimidine derivative of formula IV are as follows:

The nucleophilic displacement of the leaving group W in a compound of formula II where X is CH₂ with a purine derivative of formula III or pyrimidine derivative of formula IV as described by H Kapeller, H Baumgartner and H Griengl, Monattsh Chem,1997, 128, 191 and P Wang et al, Tet Lett 1997, 38, 4207.

The reaction of a cyclopentane derivative of formula II in which W is OH with a purine derivative under Mitsonobu conditions, which employs a triarylphosphine such as triphenyl phosphine and a diazodicarboxylic acid diester such as diethyl azodicarboxylate as reagents, as described by T Jenny et al Helv Chim Acta 1992, 25, 1944.

Such methods often result in mixtures of anomeric nucleoside derivatives which can be separated by standard techniques known to the art such as recrystallisation, column chromatography, high performance liquid chromatography or super critical fluid chromatography.

The purine derivatives of formula III and pyrimidines derivatives of formula IV for above condensation reactions can be obtained commercially or can be prepared by procedures known to the art.

The preparation of purine derivatives of formula III is reviewed by G Shaw in “Comprehensive Heterocyclic Chemistry” pub Pergamon Press Vol. 5 chapter 4.09, p 499 and “Comprehensive Heterocyclic Chemistry II” pub Pergamon Press Vol 7, chapter 7.11 p 397.

The preparation of pyrimidines derivatives of formula IV is reviewed by D J Brown “The Chemistry of Heterocyclic Compounds—The Pyrimidines” 1962 and Supplement 1, 1970, pub John Wiley and Sons, New York, by D J Brown in “Comprehensive Heterocyclic Chemistry” pub Pergamon Press Vol. 5 chapter 4.09, p 499 and by K Unheim and T Benneche in “Comprehensive Heterocyclic Chemistry II” pub Pergamon Press Vol. 6 chapter 6.02 p 93.

For example the appropriate purine base of formula III may be prepared from the corresponding purine wherein the 2, 6 or 8 position of the purine base is substituted with a suitable leaving group such as halogen or sulphonate. Such purine precursors bearing leaving groups are available commercially e.g. 6-chloropurine (Aldrich Chemical Company), 2,6-dichloropurine (Aldrich Chemical Company), 2-chloro-6-aminopurine (Aldrich Chemical Company), 8-bromoadenine (Sigma-Aldrich Company Limited) or obtained by procedures known in the art. For example 2- and 6-chloro substituted purines can be prepared by chlorination of the corresponding 2 and 6-hydroxypurines respectively by the use of chlorinating agents such as phosphorus oxychloride (D S Bakuni et al Indian J Chem Sect B 1984, 23, 1286; M P LaMontagne et al J Heterocycl Chem 1983, 20, 295) while introduction of a bromine into the 8-position of purines can be accomplished by direct bromination using brominating agents such as for example bromine (M Mano et al, Chem Pharm Bull 1983,31, 3454) or N-bromosuccinimide (J L Kelley et al J Heterocycl Chem 1990,27,1505). The purines where the 6 substituent is alkoxy, aryloxy, SH, alkylthio, arylthio, alkylamino, cycloalkylamino, saturated cyclic amino, nitrogen linked heteroaromatic, hydroxylamino, alkoxylamino, hydrazine, alkylhydrazino may be prepared by treatment of the corresponding 6-halopurine with the appropriate alkoxides, thiols, amines, nitrogen containing heterocycles, hydroxylamines and hydrazines, (e g M-Y Chae et al J Med Chem, 1994, 37, 342; G Niebch and F Schneider, Z.Naturforsch. B. Anorg. Chem. Org. Chem. Biochem. Biophys. Biol. 1972,27, 675; M P LaMontagne et al, J Heterocycl Chem 1983, 20, 295; K G Estep et al J Med Chem 1995, 38, 2582). Similarly 2-substitued purines can be prepared from the corresponding 2-halopurine for example purines where the 2 substituent is alkoxy, aryloxy, SH, alkylthio, arylthio or NR⁷R⁸ can be prepared from the corresponding 2-halopurine by treatment with alkoxides, thiols or amines (e.g. G B Barlin and D M Fenn, Aust J Chem, 1983, 36, 633; D A Nugiel et al, J Org Chem, 1997, 62, 201). Similarly 8-substitued purines can be prepared from the corresponding 8-halopurine. For example purines where the 8-substituent is alkoxy, aryloxy, SH, alkylthio, arylthio or NR⁷R⁸ can be prepared by treatment of the corresponding 8-bromopurine with the appropriate alkoxides, thiols or amines (Xing et al, Tet Lett, 1990, 31, 5849; M Mano et al, Chem Pharm Bull 1983,31, 3454). Where the 2, 6 or 8 substituent is a cyclic amine moiety the purine can be prepared from the 6-aminopurine by reaction with an appropriate dialkylating agent such as a dihaloalkane. In some cases where the 6-substituent is a nitrogen containing heteroaromatic linked through the nitrogen atom the purine may be prepared from the 6-aminopurine by reaction with a dicarbonyl compound or a reactive derivative of this such as an acetal. For example 6-(1H-pyrrol-1-yl)-1H-purine can be prepared from 6-chloropurine by reaction with 2,5-dimethoxytetrahydrofuran as described by K G Estep et al J Med Chem 1995, 38, 2582.

The furanose and thiofuranose derivatives of formula II used for the condensation reactions can be prepared by methods known in the art of carbohydrate chemistry.

Furanose derivatives can be prepared from commercially available carbohydrate starting materials such as the D or L forms of ribose, arabinose, xylose or lyxose. Following introduction of protecting groups which are compatible with the chemistry, modification of either the 2-hydroxy substituent or 3-hydroxy substituent is possible. For example direct alkylation with alkylating agents such as alkyl halides, alkyl sulphonates or diazoalkanes provides the corresponding O-alkyl derivatives as exemplified by M E Jung, C Castro, S I Khan, Nucleosides and Nucleotides; 1998, 17, 2383; G Parmentier, G Scmitt, F Dolle, B Luu Tet 1994, 50, 5361. Conversion of either hydroxy to a leaving group such as halo followed by reduction provides the 2- or 3-deoxysugar derivatives as described by K C Nicolaou et al J Am Chem Soc 1988, 110,4672. Also conversion of either hydroxy to a leaving group such as halo or sulphonate by standard methods followed by displacement with nucleophilic reagents for example sodium or lithium azide to introduce an azido group (A M Ozols et al, Synthesis, 1980, 557). Direct introduction of a fluorine substituent can be accomplished with fluorinating agents such as diethylaminosulphur trifluoride as described by F Puech, G Gosselin and J-L Imbach Tet Lett 1989, 30, 3171 or conversion of the hydroxy substituent to a leaving group such as halo or sulphonate and displacement using reagents such as tetrabutylammonium fluoride as described in Tet Asym 1990,1 715.

3′-Alkyl substituted furanoses can be prepared by construction of the sugar ring from γ-hydroxymethyl-y-butyrolactone as described by K Ayei-Aye and D C Baker, Carbohydr Res 1988, 183, 261 and by M Okabe et al J Org chem, 1988, 53, 4780. Alternatively, cyclohexenecarboxylic acid derivatives can be used as described by K C Schneider and S A Benner, Tet Lett, 1990, 31, 335.

3′-hydroxymethyl substituted furanoses can been synthesised from 3-[[(4-bromobenzyl)oxy]methyl]oxirane-2-methanol as described by L Svansson et al, J Org Chem 1991, 56,2993.

2,2-Difluorofuranose derivatives can be prepared from D-glucose or D-mannose as described by R Fernandez, M I Mateu, R Echarri and S Castillon Tet 1998, 54, 3523. The thiofuranose derivatives of formula II where X is S can be prepared by literature procedures such as L Bellon, J L Barascut, J L Imbach Nucleosides and Nucleotides 1992, 11, 1467 and modified in a similar fashion to the furanose analogues described above.

The cyclopentane derivatives of formula II where X is CH₂ can be prepared by methods known in the art of organic chemistry and by methods and references included in L Agrofolio et al Tetrahedron 1994, 50, 10611.

Construction of the heterocyclic base after glycosylation.

Such methods include:

those which for example utilise furanosylamine derivatives as described by N J Cusack, B J Hildick, D H Robinson, P W Rugg and G Shaw J C S Perkin I 1973, 1720 or G Shaw, R N Warrener, M H Maguire and R K Ralph, J Chem Soc 1958, 2294.

those which utilise for example furanosylureas for pyrimidine nucleoside synthesis as described by J \check{S}mejkal, J Farkas, and F \check{S}orm Coll Czech Chem Comm 1966, 31, 291.

The preparation of purine nucleosides from imidazole nucleosides as reviewed by L B Townsend Chem Rev 1967, 67, 533.

the preparation of compounds of formula I wherein X is CH₂ can be accomplished from 1-hydroxymethyl-4-aminocyclopentane derivatives as described by Y F Shealy and J D Clayton J Amer Chem Soc 1969, 91, 3075;R Vince and S Daluge J Org Chem 1980, 45, 531;R C Cermak and R Vince Tet Lett 1981,2331;R D Elliott et al J Med Chem 1994,37, 739; A D Borthwick et al, J Med Chem 1990, 33, 179.

Modification or inter-conversion of preformed nucleosides.

Modification of the purine or pyrimidine base moiety.

Methods include:

the deamination of aminopurine or aminopyrimidine nucleosides as described by J R Tittensor and R T Walker European Polymer J 1968, 4, 39 and H Hayatsu Progress in Nucleic Acid Research and Molecular Biology 1976, Vol. 16, p75.

The conversion of the 4-hydroxy group of 4-hydroxypyrimidine nucleosides to a leaving group and displacement with nucleophilic reagents. Such leaving groups include halogen as described by J Brokes and J Beranek Col Czech Chem Comm 1974, 39, 3100 or 1,2,4-triazole as described by K J Divakar and C B Reece J Chem Soc Perkin Trans I 1982, 1171.

5-substitution of pyrimidine nucleosides has been achieved by the use of 5-metallo derivatives such as 5-mercuri or 5-palladium for example as described by D E Bergstrom and J L Ruth J Amer Chem Soc 1976, 98, 1587. Introduction of fluoro into the 5 position of pyrimidine nucleosides can be achieved with reagents such as trifluoromethyl hypofluorite as described by M J Robins Ann New York Acad Sci 1975, 255, 104.

modified purine nucleosides may be prepared from the corresponding purine nucleoside derivatives wherein the 2, 6 or 8 substituent is a suitable leaving group such as halogen or sulphonate or 1,3,4-triazole. Thus the compounds for example where the purine 6 substituent is alkoxy, aryloxy, SH, alkylthio, arylthio, alkylamino, cycloalkylamino hydroxylamino, alkoxylamino or hydrazino may be prepared by treatment of the appropriate 6-halopurine or 6-(1,2,4-triazol-4-yl)purine nucleoside derivatives with the appropriate alcohols, thiols or amines, hydroxylamines or hydrazines. Such conversions are described by V Nair and A J Fassbender Tet 1993,49,2169 and by V Samano, R W Miles and M J Robins J Am Chem Soc 1994, 116, 9331. Where the 6 substituent is a cyclic amine or aromatic amine moiety the purine nucleoside analogue can be prepared from the 6-aminopurine nucleoside derivative by reaction respectively with an appropriate dialkylating agent such as a dihaloalkane or with a dicarbonyl compound or a reactive derivative of this such as an acetal. For example as described by M Haidoune and R Mornet J Heterocyclic Chem 1995, 31,1462. Similarly 8-substituted purine nucleosides can be prepared by treatment of the corresponding 8-halopurine nucleoside with the appropriate nucleophilic reagent for example alkoxides, thiols or amines as described by L Tai-Shun, C Jia-Chong, I Kimiko and A C Sartorelli J Med Chem 1985, 28, 1481; Nandanan et al J Med Chem 1999,42,1625; J Jansons, Y Maurinsh, and M Lidaks Nucleosides and Nucleotides 1995, 14, 1709. Introduction of a 8-cyano substituent can be accomplished by displacement of using a metal cyanide as described by L-L Gundersen, Acat Chem Scand 1996, 50, 58. 2-modified purine nucleoside may be prepared in a similar fashion as described by T Steinbrecher, C Wamelung, F Oesch and A Seidl Angew Chem Int Ed Engl 1993, 32, 404.

Where the substituent at the 2, 6 or 8-position of the purine nucleoside is linked via a carbon carbon bond e.g. alkyl or aryl then metal catalysed cross-coupling procedures can be used starting with the appropriate 2, 6 or 8-halosubstituted purine nucleoside analogue. Such procedures are described by A A Van Aerschott, et al J Med Chem 1993, 36, 2938; D E Bergstrom and P A Reday Tet Lett 1982, 23, 4191.M Hocek, A Holy, I Votruba and H Dvarakova J Med Chem 2000, 43, 1817.C Tu, C Keane and B E Eaton Nucleosides and Nucleotides 1995, 14, 1631.

Oxidation of the 3-nitrogen in pyrimidine nucleoside analogues or 1-nitrogen in purine nucleoside derivatives can be accomplished using hydrogen peroxide or organic peroxides as described by G B Brown Progress in Nucleic Acid Research and Molecular Biology ed J N Davidson and W E Cohn, Academic Press, New York 1968,8,209.

Alkylation of the 3-nitrogen in uracil nucleoside analogues can be accomplished using alkylating agents such as diazoalkanes (Miles, Biochim Biophys Acta, 1956, 22, 247), alkyl sulphonates (Scannel et al, Biochim Biophys Acta, 1959, 32, 406) or alkyl halides (Anderson et al J Chem Soc 1952, 369). Alkylation of the 3-nitrogen in cytosine nucleoside analogues can similarly be accomplished using alkylating agents such as trialkyl sulphonium halides (K Yamauchi, J Chem Soc Perkin Trans 1, 1980, 2787) or epoxides (W Zhan et al Chem Res Toxicol, 1998, 8, 148). Similarly alkylation of purine nucleoside analogues on the 1-nitrogen can be accomplished using alkylating agents such as alkyl halides (W A Szarek et al Can J Chem 1985, 63, 2149) or alkyl sulphonates (M Kawana et al J Chem Soc Perkin Trans 1, 1992, 4, 469). Aryl substituents can be introduced onto the 1-nitrogen of purine nucleosides or the 3-nitrogen of pyrimidine nucleosides by direct arylation using aryl halides in the presence of a copper catalyst such as copper(I) oxide as described for example by T Maruyama et al, Nucleosides and Nucleotides, 1997, 16, 1079 and by T Maruyama et al J Chem Soc Perkin Trans I, 1995, 733.

B. Modification of the carbohydrate moiety.

Methods include:

Following introduction of protecting groups which are compatible with the further chemistry, modification of either the 2′-hydroxy substituent or 3′-hydroxy substituent in the nucleoside analogue is possible. For example direct alkylation with alkylating agents such as alkyl halides, alkyl sulphonates or diazoalkanes provides the corresponding O-alkyl derivatives as exemplified by C G Edmonds et al J Chem Soc Chem Comm 1987, 12, 909; P J L M Quaedfieg et al J Org Chem 1991, 56, 5846. Conversion of either hydroxy to a leaving group such as halo by reaction with for example triphenyl phosphine and a tetrahaloalkane as described for example by L De Napoli et al, Nucleosides and Nucleotides, 1993, 12, 981, followed by reduction provides the 2- or 3-deoxysugar derivatives as described by D G Norman and C B Reese, Synthesis 1983, 304. Alternatively derivatisation of the hydroxy function by conversion to a thiocarbonate group such as phenoxy thiocarbonate or imidazoylthiocarbonate followed by reduction using free radical reducing agents such as trialkyltin hydrides as described by D H R Barton and R Subranian J Chem Soc Chem Comm 1976, 867. Direct introduction of a fluorine substituent can be accomplished with fluorinating agents such as diethylaminosulphur trifluoride as described by P Herdewijn, A Van Aerschot and L Kerremans Nucleosides and Nucleotides 1989,8, 65. Conversion of the hydroxy substituent to a leaving group such as halo or sulphonate also allows displacement using nucleophilic reagents such as tetrabutylammonium fluoride, lithium azide, tert butyl isocyanide or metal cyanides as exemplified by H Hrebabecky, A Holy and e de Clercq Collect Czech Chem Comm 1990, 55, 1800; K E B Parkes and K Taylor Tet Lett 1988, 29, 2995. Such nucleophilic reactions can also be carried out on 2′,3′-epoxynucleosides as exemplified by Huang et al J Med Chem 1991, 34, 1640 or using 2,3′-anhydropyrimidine nucleosides as typified by Colla et al Eur J Med Chem Chim Ther 1985, 20, 295.

Following introduction of appropriate protecting groups on the 3′ and 5′-hydroxy groups of a preformed nucleoside it is possible to oxidise the unprotected 2′-hydroxy group to a ketone using methods similar to those described by F Hansske, M D Fritz and M J Robins, Tetrahedron 1984, 40, 125.Reaction of the resultant 2′-keto nucleoside with olefination reagents such as methyl triphenyl phosphonium bromide in the manner of S Czernecki, L Mulard, J-M Valery, and A Commercon, Can.J.Chem 1993, 71, 413 provides the 2′-deoxy-2′-methylidenenucleoside derivatives.

Reaction of 2′-keto nucleosides with fluorinating agents such as diethylamino sulfur trifluoride can be used to prepare 2′,2′-difluoronucleosides as described by D Bergstrom, E Romo and P Shum Nucleosides and Nucleotides 1987, 6,53.

The principal methods of introducing an alkyl group into the 3′-position of nucleosides involve, free-radical coupling of protected nucleosides which are suitably derivatised in the 3′-position, for example from 3′-iodonucleosides as described by D Yu and M d'Alarco, J Org Chem 1989,54,3240 or from 3′-O-phenoxythiocarbonyl nucleosides as described by J Fiandor and S Y Tam, Tet Lett, 1990,31, 597 and C K Chu et al, J Org Chem, 1989,54, 2767, or through addition of cyanide to 3′-ketonucleosides as described by M J Camarasa et al, J Med Chem, 1989, 32, 1732. A 3′-hydroxymethyl substituent can be introduced by reduction of the corresponding 3′-C-formyl nucleoside as described by M J Bamford et al, J Med Chem, 1990, 33, 2494. The 3′-C-formyl nucleoside can be produced in turn by elaboration of 3′-keto nucleosides or from 2′,3′-anhydronucleosides.

The preformed nucleoside derivatives are either available commercially or synthesised in accordance with the methods described above.

Also part of this invention are novel purine and pyrimidine nucleoside derivatives, a process for their manufacture, pharmaceutical compositions and the use of such compounds in medicine. In particular, the compounds are useful as inhibitors of subgenomic Hepatitis C Virus (HCV) RNA replication and pharmaceutical compositions of such compounds.

The novel compounds of this invention are novel purine and pyrimidine nucleoside derivatives listed as follows:

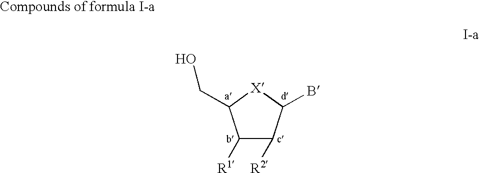

wherein

R¹′ is hydroxy;

R²′ is hydroxy;

X is O;

a′, b′, c′, d′ denoting asymmetric carbon atoms and forming a D-ribofuranosyl ring; and

B′ signifies an oxidised purine base B2-a which is connected through the 9-nitrogen of formula

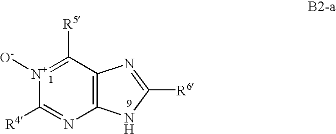

wherein

R⁴′ is hydrogen;

R⁵′ is NHR⁸′;

R⁶′ is hydrogen;

R⁸′ is alkyl,preferably wherein

R⁸′ is methyl, ethyl, propyl, isopropyl, n-butyl, isobutyl, tert.-butyl, phenylmethyl (benzyl), 1-phenylethyl, 2-phenylethyl, 1(S)-methyl-2-phenylethyl, 1(R)-methyl-2-phenylethyl, 1-phenylpropyl, 2-phenylpropyl or 3-phenylpropyl; hydrolyzable esters or ethers thereof and pharmaceutically acceptable salts thereof.

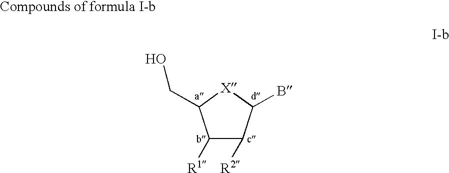

wherein

R¹″ is hydroxy;

R²″ is hydroxy;

X″ is O;

a″, b″, c″, d″ denoting asymmetric carbon atoms and forming a D-ribofuranosyl ring; and

B″ signifies a purine base B3-a which is connected through the 9-nitrogen of formula

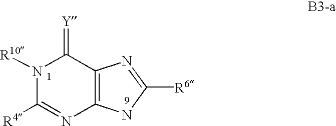

wherein

R⁴″ is hydrogen;

R⁶″ is hydrogen;

R¹⁰″ is alkyl, preferably wherein

R¹¹″ is methyl, ethyl, propyl, isopropyl, n-butyl, isobutyl, tert.-butyl;

Y″ is NR¹¹″;

R¹¹″ is alkyl, preferably wherein

R¹¹″ is methyl, ethyl, propyl, isopropyl, n-butyl, isobutyl, tert.-butyl, phenylmethyl (benzyl), 1-phenylethyl, 2-phenylethyl, 1(S)-methyl-2-phenylethyl, 1(R)-methyl-2-phenylethyl, 1-phenylpropyl, 2-phenylpropyl or 3-phenylpropyl; hydrolyzable esters or ethers thereof and pharmaceutically acceptable salts thereof.

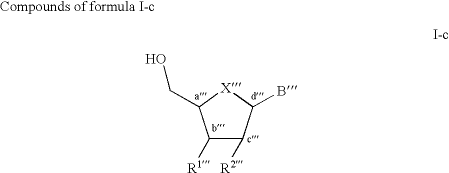

wherein

R¹′″ is hydroxy;

R²′″ is hydroxy;

X′″ is O;

a′″, b′″, c′″, d′″ denoting asymmetric carbon atoms and forming a D-ribofuranosyl ring; and

group B′″ signifies a pyrimidine base B4-a which is connected through the 1-nitrogen of formula

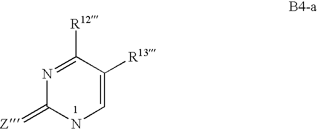

wherein

R¹²′″ is alkylthio or heterocyclyl, preferably wherein

R¹²′″ is methylthio, ethylthio, n-propylthio, i-propylthio, n-butylthio, i-butylthio, tert.-butylthio or oxazolyl, isoxazolyl, furyl, tetrahydrofuryl, 2-thienyl, 3-thienyl, pyrazinyl, isothiazolyl, indolyl, didehydroindolyl, indazolyl, quinolinyl, pyrimidinyl, benzofuranyl, 1-pyrrolidinyl, 2-pyrrolidinyl, 3-pyrrolidinyl, 1-pyrrolyl, 2-pyrrolyl, triazolyl e.g. 1,2,3-triazolyl or 1,2,4-triazolyl, 1-pyrazolyl, 2-pyrazolyl, 4-pyrazolyl, benzotriazolyl, piperidinyl, morpholinyl (e.g. 4-morpholinyl), thiomorpholinyl (e.g. 4-thiomorpholinyl), thiazolyl, pyridinyl, dihydrothiazolyl, imidazolidinyl, pyrazolinyl, benzothienyl, piperazinyl, 1-imidazolyl, 2-imidazolyl, 4-imidazolyl, thiadiazolyl e.g. 1,2,3-thiadiazolyl, 1,2,3,4-tetrahydroquinoline, 1,2,3,4-tetrahydroisoquinoline, benzothiazolyl;

R¹³′″ is hydrogen, alkyl or halogen, preferably wherein

R¹³′″ is methyl, ethyl, propyl, isopropyl, n-butyl, isobutyl, tert.-butyl or fluorine, chlorine, bromine or iodine;

Z′″ is O;

hydrolyzable esters or ethers thereof and pharmaceutically acceptable salts thereof.

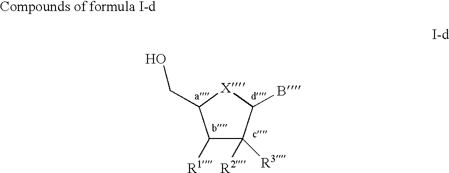

wherein

R¹″″ is hydrogen, halogen, hydroxy, alkyl, alkoxy, cyano or azido, preferably wherein

R¹″″ is hydrogen, fluorine, hydroxy, C₁₋₄-alkyl, C₁₋₄-alkoxy, cyano or azido, more preferred wherein

R¹″″ is hydrogen, fluorine, hydroxy, C₁₋₄-alkyl or C₁₋₄-alkoxy, and most preferred wherein

R¹″″ is hydroxy;

R²″″ and R³″″ represent fluorine;

X″″ is O or CH₂, preferably wherein

X″″ is CH₂;

a″″, b″″, c″″, d″″ denoting asymmetric carbon atoms each of which is substituted with 4 different substituents; and

group B″″ signifies a pyrimidine base B4-b which is connected through the 1-nitrogen of formula

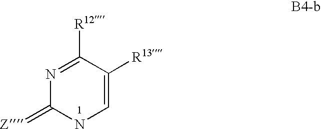

wherein

Z″″ is O;

R¹²″″ is NR⁷″″R⁸″″, preferably wherein

R¹²″″ is hydrogen, alkyl or halogen;

R¹³″″ is hydrogen, alkyl or halogen, preferably wherein

R¹³″″ is hydrogen, C₁₋₄-alkyl or fluorine, more preferred wherein

R¹³″″ is hydrogen, methyl, ethyl or fluorine, and most preferred wherein

R¹³″″ is hydrogen;

R⁷″″ and R⁸″″ are independently of each other hydrogen or alkyl, preferably wherein

R⁷″″ and R⁸″″ are independently of each other hydrogen or C₁₋₄-alkyl, more preferred wherein

R⁷″″ and R⁸″″ are independently of each other hydrogen, methyl or ethyl, and most preferred wherein

R⁷″″ and R⁸″″ are independently of each other hydrogen; hydrolyzable esters or ethers thereof and pharmaceutically acceptable salts thereof.

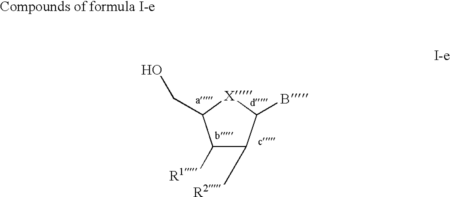

wherein

R¹′″″ is alkoxy, preferably wherein

R¹′″″ is methoxy, ethoxy, n-propyloxy, i-propyloxy, n-butyloxy, i-butyloxy, tert.-butyloxy;

R²′″″ is hydrogen;

X′″″ is O;

a′″″, b′″″, c′″″, d′″″ denoting asymmetric carbon atoms and forming a D-ribofuranosyl ring; and

group B′″″ signifies a pyrimidine base B5-a which is connected through the 1-nitrogen of formula

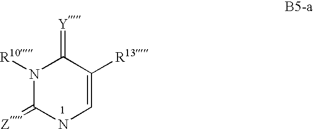

wherein

R¹⁰′″″ is hydrogen;

R¹³′″″ is alkyl, preferably wherein

R ¹³′″″ is methyl, ethyl, propyl, isopropyl, n-butyl, isobutyl, tert.-butyl;

Y′″″ is O;

Z′″″ is O;

hydrolyzable esters or ethers thereof and pharmaceutically acceptable salts thereof.

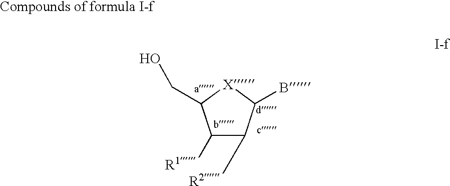

wherein

R¹″″″ is hydroxy;

R²″″″ is hydroxy;

X″″″ is O;

a″″″, b″″″, c″″″, d″″″ denoting asymmetric carbon atoms and forming a D-ribofuranosyl ring; and

group B″″″ signifies a pyrimidine base B5-b which is connected through the 1-nitrogen of formula

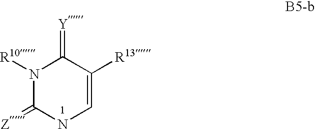

wherein

R¹⁰″″″ is hydrogen;

R¹³″″″ is halogen, preferably wherein

R¹³″″″ is fluorine, chlorine or bromine;

R¹³″″″ is hydroxy;

Z″″″ is O;

hydrolyzable esters or ethers thereof and pharmaceutically acceptable salts thereof.

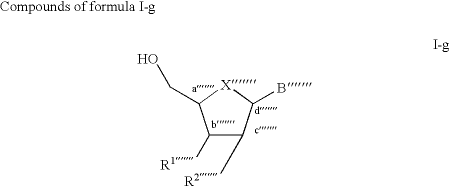

wherein

R¹′″″″ is hydroxy;

R²′″″″ is hydroxy;

a′″″″, b′″″″′″″″″ is , c′″″″, d′″″″ denoting asymmetric carbon atoms and forming a L-ribofuranosyl ring; and

group B′″″″ signifies a pyrimidine base B5-c which is connected through the 1-nitrogen of formula

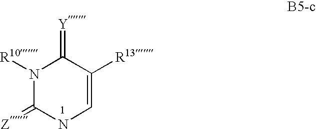

wherein

R¹⁰′″″″ is hydrogen;

R¹³′″″″ is hydrogen;

Y′″″″ is NR¹¹′″″″;

R¹¹′″″″ is hydroxy;

Z′″″″ is O;

hydrolyzable esters or ethers thereof and pharmaceutically acceptable salts thereof.

The terms as they are used for the novel purine and pyrimidine nucleoside derivatives are as defined above.

More preferred embodiments of compounds of formula I hydrolyzable esters or ethers thereof and pharmaceutically acceptable salts thereof, are listed in table 2:

TABLE 2

| STRUCTURE | SYSTEMATIC NAME |
|---|---|
| 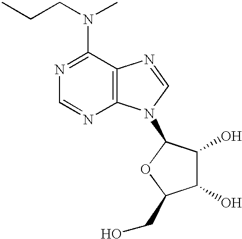 | 6-(N-Methylpropylamino)-9-(β-D-ribo- furanosyl)purine |
| 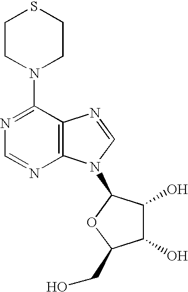 | 9-(β-D-Ribofuranosyl)-6-(4- thiomorpholinyl)purine |
| 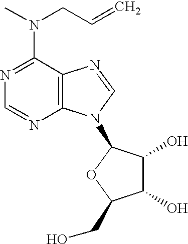 | 6-(N-Methyl-2-propenylamino)-9-(β-D-ribo- furanosyl)purine |
| 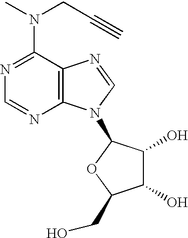 | 6-(N-Methyl-2-propynylamino)-9-(β-D-ribo- furanosyl)purine |
| 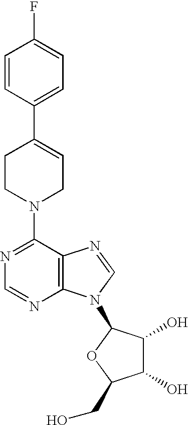 | 6-[4-(4-Fluorophenyl)-1,2,5,6-tetrahydropyridyl]-9-(β-D-ribo- furanosyl)purine |
| 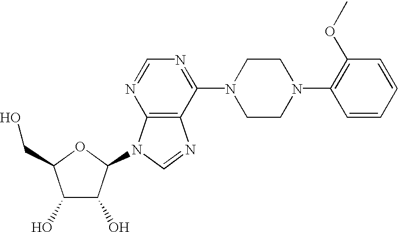 | 6-[4-(2-Methoxyphenyl)piperazinyl]-9-(β-D-ribo- furanosyl)purine |
| 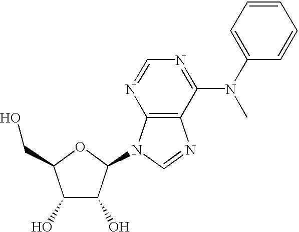 | 6-(N-Methylphenylamino)-9-(β-D-ribo- furanosyl)purine |
| 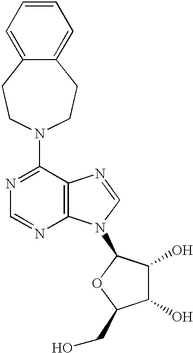 | 9-(β-D-Ribofuranosyl)-6-(1,2,4,5-tetrahydro -3H-benzazepin-3-yl)purine |
| 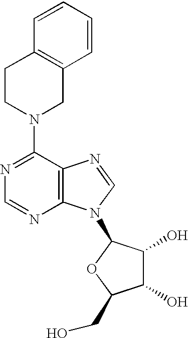 | 9-(β-D-Ribofuranosyl)-6-(1,2,3,4-tetrahydro-2-iso- quinolyl)purine |
| 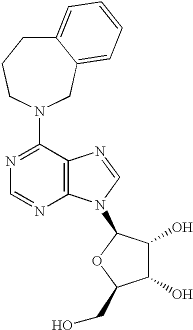 | 9-(β-D-Ribofuranosyl)-6-(1,3,4,5-tetrahydro-2H-ben- zazepin-2-yl)purine |
| 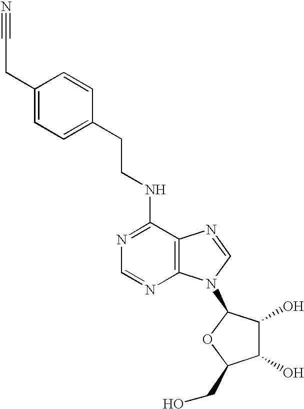 | 6-[2-(4-Cyanomethylphenyl)ethylamino]-9-(β-D-ribo- furanosyl)purine |
| 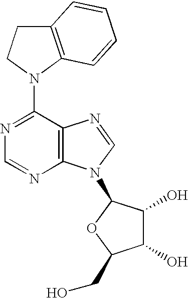 | 6-(2,3-Dihydro-1-indolyl)-9-(β-D-ribo- furanosyl)purine |
| 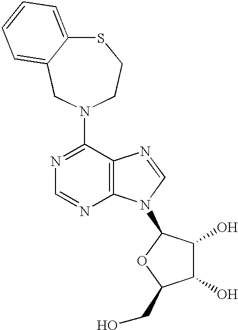 | 9-(β-D-Ribofuranosyl)-6-(2,3,4,5-tetrahydro-1,4-benzo- thiazepin-4-yl)purine |
| 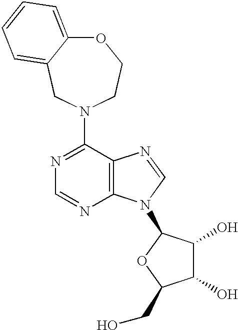 | 9-(β-D-Ribofuranosyl)-6-(2,3,4,5-tetrahydro-1,4- benzoxazepin-4-yl)purine |
| 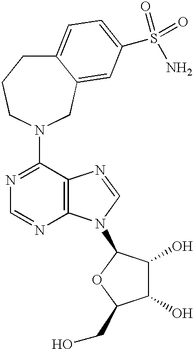 | 6-(8-Aminosulphonyl-2,3,4,5-tetrahydro-1H-2-ben- zazepin-2-yl)-9-(β-D-ribofuranosyl)purine |
| 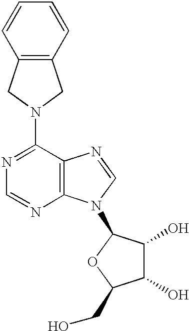 | 6-(2-Isoindolinyl)-9-(β-D-ribofuranosyl)purine |
| 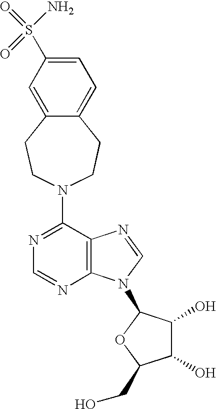 | 6-(7-Aminosulphonyl-2,3,4,5-tetrahydro-1H-ben- zazepin-3-yl)-9-(β-D-ribofuranosyl)purine |
| 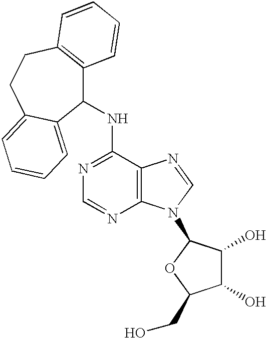 | 6-(10,11-Dihydro-5H-dibenzo[a,d]cyclohepten-5- ylamino)-9-(β-D-ribofuranosyl)purine |
| 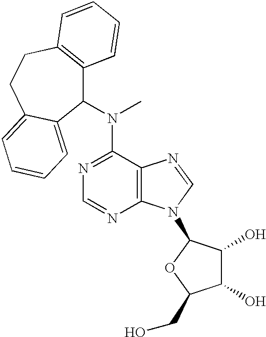 | 6-[N-(10,11-Dihydro-5H-dibenzo[a,d]cyclo- hepten-5-yl)methylamino]-9-(β-D-ribofuranosyl)purine |
| 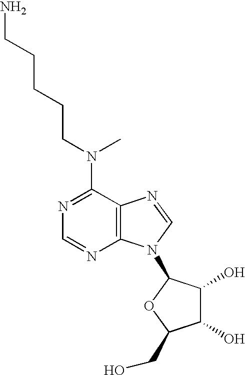 | 6-[N-(5-Aminopentyl)methylamino]-9-(β-D-ribo- furanosyl)purine |
| 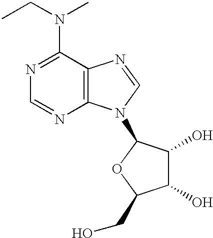 | 6-Ethylmethylamino-9-(β-D- ribofuranosyl)purine |
| 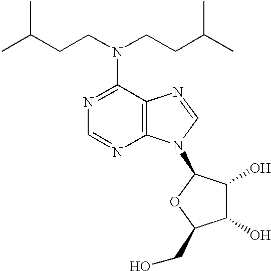 | 6-bis-[(3-Methyl)butylamino]-9-(β-D-ribo- furanosyl)purine |
| 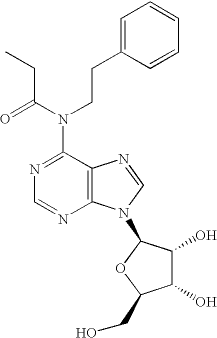 | 6-[2-Phenyl-(N-propionyl)ethylamino]-9-(β-D-ribo- furanosyl)purine |
| 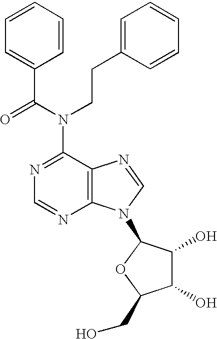 | 6-(N-Benzoyl-2-phenylethylamino)-9-(β-D-ribo- furanosyl)purine |
| 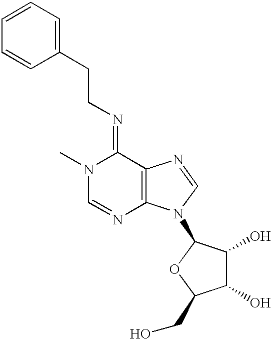 | 1-Methyl-6-(2-phenylethylimino)-9-(β-D-ribo- furanosyl)purine |
| 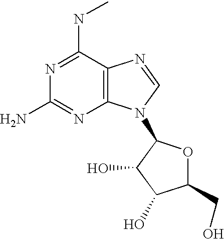 | 2-Amino-6-methylamino-9-(β-L-ribo- furanosyl)purine |
| 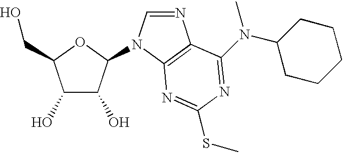 | 6-[(N-Cyclohexyl)methylamino]-2-methylthio-9-(β-D-ribo- furanosyl)purine |
| 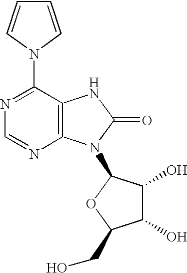 | 6-(1-Pyrrolyl)-9-(β-D-ribo- furanosyl)purin-8-(7H)-one |
| 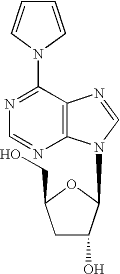 | 9-(3-Deoxy-β-D-ribo- furanosyl)-6-(1-pyrrolyl)purine |
| 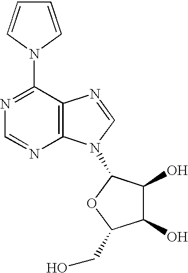 | 6-(1-Pyrrolyl)-9-(β-L-ribofuranosyl)purine |
| 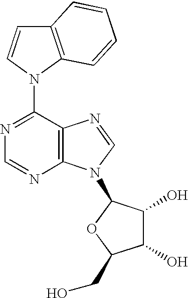 | 6-(1-Indolyl)-9-(β-D-ribofuranosyl)purine |
| 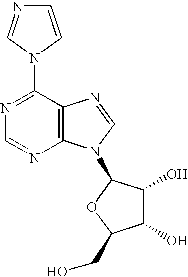 | 6-(1-Imidazolyl)-9-(β-D-ribofuranosyl)purine |
| 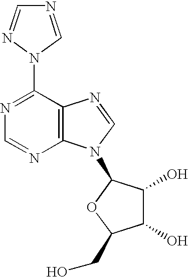 | 9-(β-D-Ribofuranosyl)-6- (1,2,4-triazol-1-yl)purine |
| 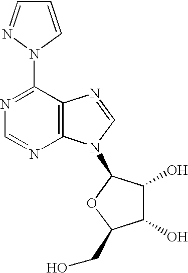 | 6-(1-Pyrazolyl)-9-(β-D-ribofuranosyl)purine |
| 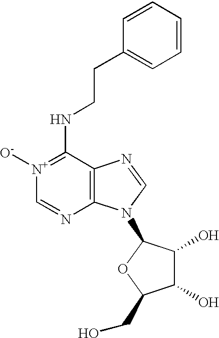 | 6-(2-Phenylethylamino)-9-(β-D-ribo- furanosyl)purine-1-oxide |
| 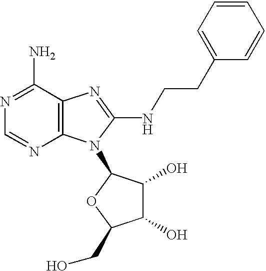 | 8-(2-Phenylethylamino)adenosine |
| 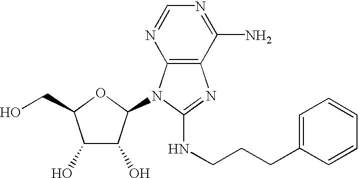 | 8-(3-Phenylpropylamino)adenosine |
| 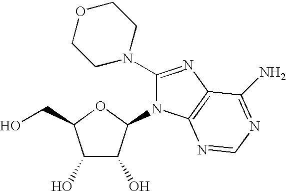 | 8-(4-Morpholinyl)adenosine |
| 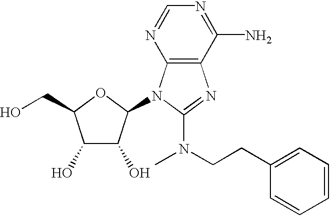 | 8-(N-Methyl-2-phenylethylamino)adenosine |
| 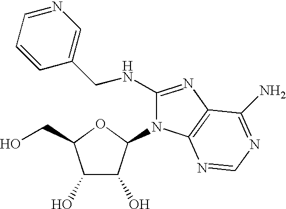 | 8-(3-Pyridylmethylamino)adenosine |
| 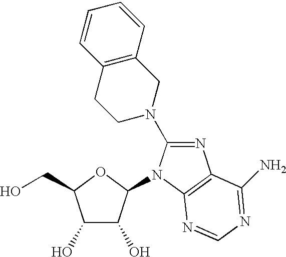 | 8-(1,2,3,4-Tetrahydro-2-isoquinolyl)adenosine |
| 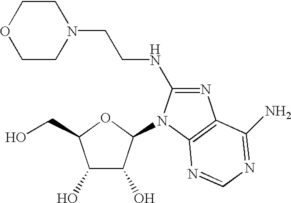 | 8-[2-(4-Morpholinyl)ethylamino]adenosine |
| 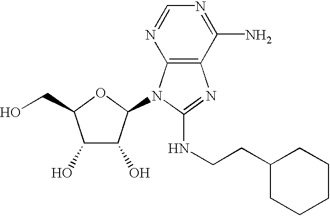 | 8-(2-Cyclohexylethylamino)adenosine |
| 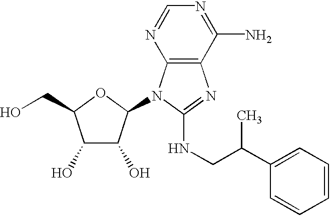 | 8-(2(R,S)-Phenylpropylamino)adenosine |
| 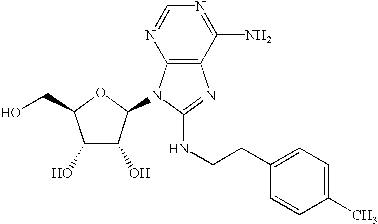 | 8-[2-(4-Methylphenyl)ethylamino]adenosine |
| 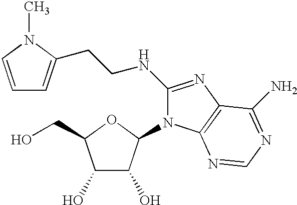 | 8-[2-(1-Methyl-2-pyrrolyl)ethyl- amino]adenosine |
| 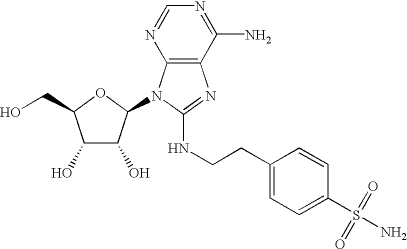 | 8-[2-(4-Aminosulphonyl- phenyl)ethylamino]adenosine |
| 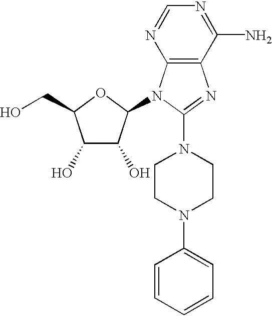 | 8-(4-Phenyl-1-piperazinyl)adenosine |
| 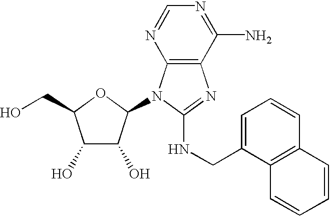 | 8-(1-Naphthylmethylamino)adenosine |
| 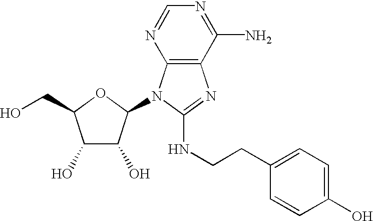 | 8-[2-(4-Hydroxyphenyl)ethylamino]adenosine |
| 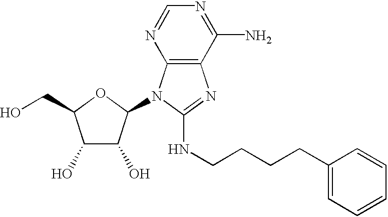 | 8-(4-Phenylbutylamino)adenosine |
| 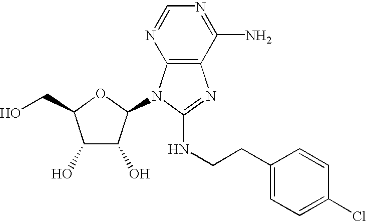 | 8-[2-(4-Chlorophenyl)ethylamino]adenosine |
| 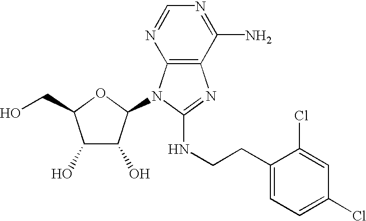 | 8-[2-(2,4-Dichlorophenyl)ethylamino]adenosine |
| 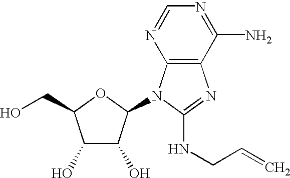 | 8-(2-Propenylamino)adenosine |
| 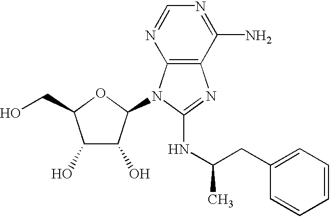 | 8-(1(R)-Methyl-2-phenylethylamino)adenosine |
| 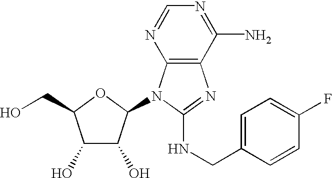 | 8-(4-Fluorobenzylamino)adenosine |
| 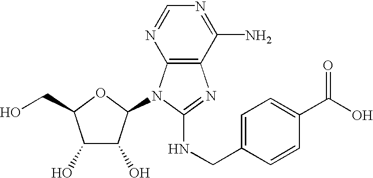 | 8-[(4-Hydroxycarbonyl)benzylamino]adenosine |
| 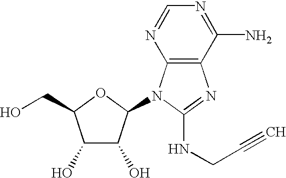 | 8-(2-Propynylamino)adenosine |
| 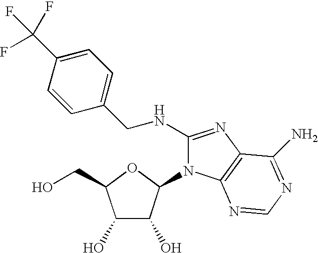 | 8-[(4-Trifluoromethyl)benzylamino]adenosine |
| 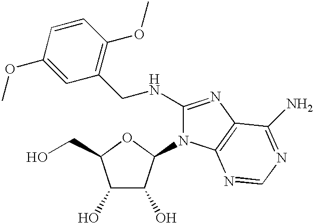 | 8-[(2,5-Dimethoxy)benzylamino]adenosine |
| 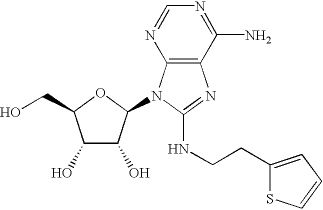 | 8-[2-(2-Thienyl)ethylamino]adenosine |
| 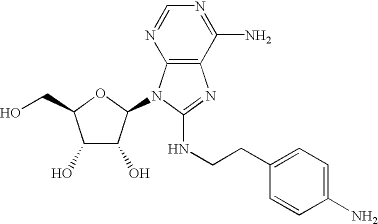 | 8-[2-(4-Aminophenyl)ethylamino]adenosine |
| 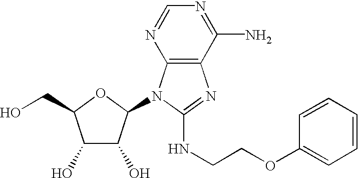 | 8-(2-Phenoxyethylamino)adenosine |
| 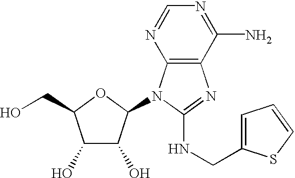 | 8-[(2-Thienyl)methylamino)adenosine |
| 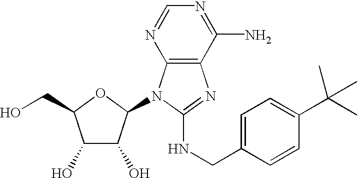 | 8-[(4-tert-Butyl)benzylamino]adenosine |
| 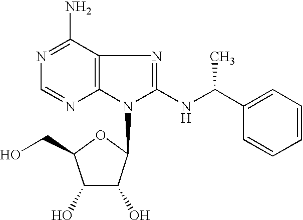 | 8-(1(R)-Phenylethylamino)adenosine |
| 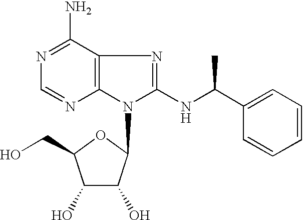 | 8-(1(S)-Phenylethylamino)adenosine |
| 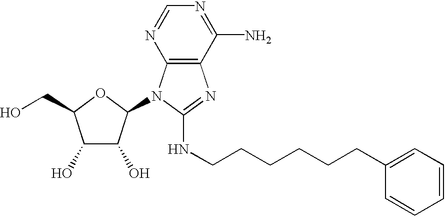 | 8-(6-Phenylhexylamino)adenosine |
| 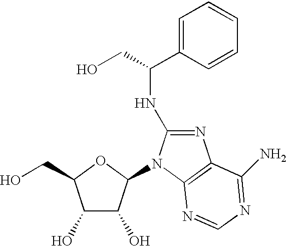 | 8-[2-Hydroxy-1(S)-phenyl)ethyl- amino]adenosine |
| 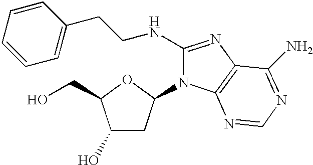 | 2′-Deoxy-8-(2-phenylethylamino)adenosine |
| 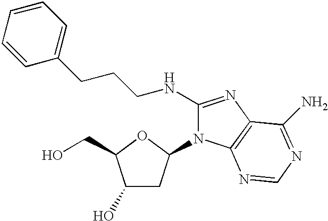 | 2′-Deoxy-8-(3-phenylpropylamino)adenosine |
| 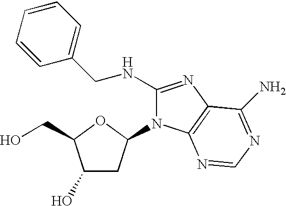 | 8-Benzylamino-2′-deoxyadenosine |
| 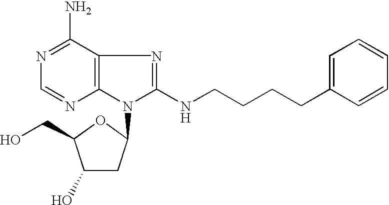 | 2′-Deoxy-8-(4-phenylbutylamino)adenosine |
| 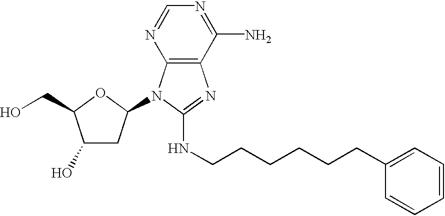 | 2′-Deoxy-8-(6-phenylhexylamino)adenosine |
| 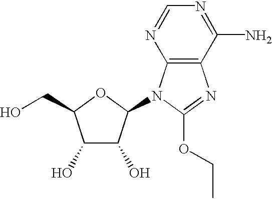 | 8-Ethoxyadenosine |
| 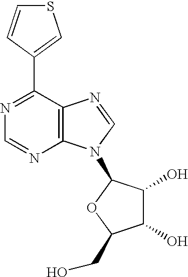 | 9-(β-D-Ribofuranosyl)-6-(3-thienyl)purine |
| 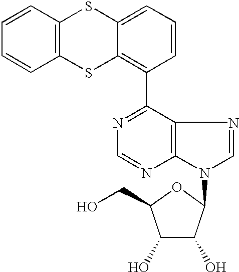 | 9-(β-D-Ribofuranosyl)-6-(1-thianthrenyl)purine |
| 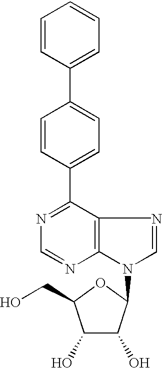 | 6-(4-Biphenylyl)-9-(β-D-ribofuranosyl)purine |
| 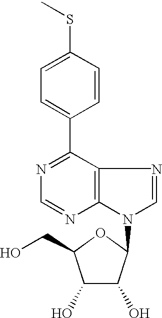 | 6-(4-Methylthiophenyl)-9-(β-D-ribo- furanosyl)purine |
| 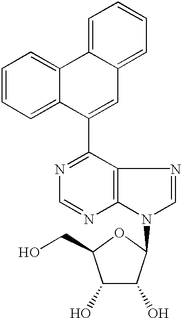 | 6-(9-Phenanthrenyl)-9-(β-D- ribofuranosyl)purine |
| 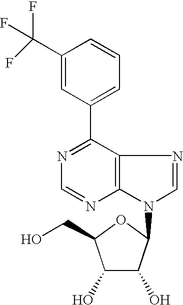 | 9-(β-D-Ribofuranosyl)-6-(3-tri- fluoromethylphenyl)purine |
| 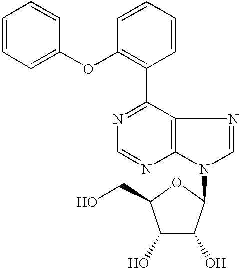 | 6-(2-Phenoxyphenyl)-9-(β-D- ribofuranosyl)purine |
| 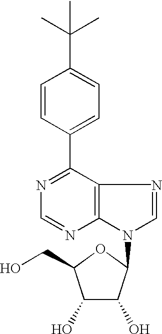 | 6-(4-tert-Butylphenyl)-9-(β-D- ribofuranosyl)purine |
| 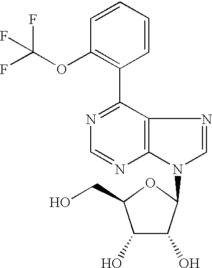 | 9-(β-D-Ribofuranosyl)-6-(2-tri- fluoromethoxyphenyl)purine |
| 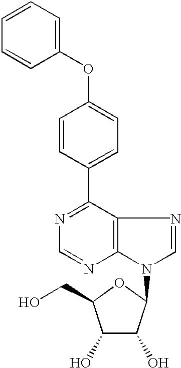 | 6-(4-Phenoxyphenyl)-9-(β-D- ribofuranosyl)purine |
| 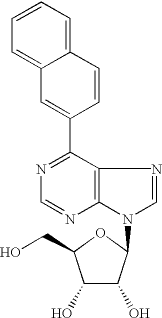 | 6-(2-Naphthyl)-9-(β-D-ribofuranosyl)purine |
| 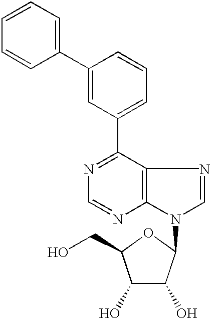 | 6-(3-Biphenylyl)-9-(β-D-ribofuranosyl)purine |
| 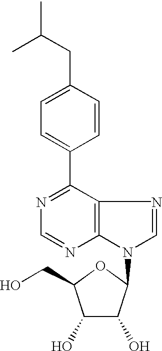 | 6-[4-(2-Methylpropyl)phenyl]-9-(β-D-ribo- furanosyl)purine |
| 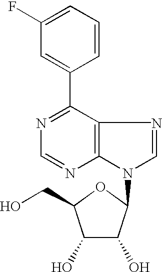 | 6-(3-Fluorophenyl)-9-(β-D- ribofuranosyl)purine |
| 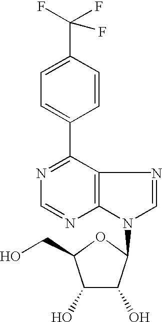 | 9-(β-D-Ribofuranosyl)-6-(4-tri- fluoromethylphenyl)purine |
| 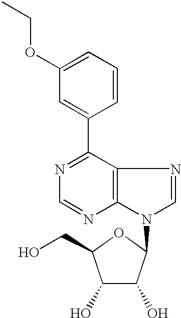 | 6-(3-Ethoxyphenyl)-9-(β-D- ribofuranosyl)purine |
| 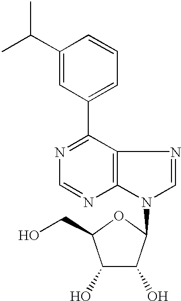 | 6-[3-(1-Methyl)ethylphenyl]-9-(β-D-ribo- furanosyl)purine |
| 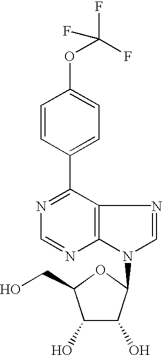 | 9-(β-D-Ribofuranosyl)-6-(4-tri- fluoromethoxyphenyl)purine |
| 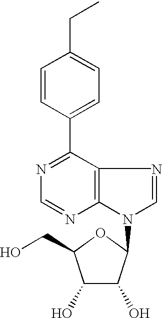 | 6-(4-Ethylphenyl)-9-(β-D-ribofuranosyl)purine |
| 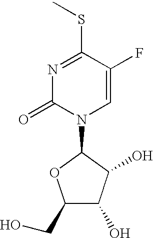 | 5-Fluoro-4-methylthio-1-(β-D-ribo- furanosyl)pyrimidin-2(1H)-one |
| 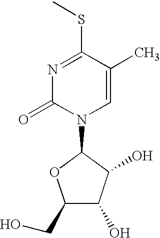 | 5-Methyl-4-methylthio-1-(β-D-ribo- furanosyl)pyrimidin-2(1H)-one |
| 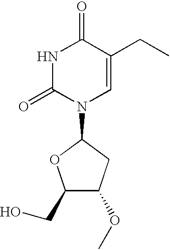 | 2′,3′-Dideoxy-5-ethyl-3′-methoxyuridine |
| 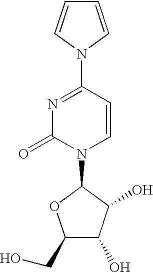 | 4-(1-Pyrrolyl)-1-(β-D-ribofuranosyl)py- rimidin-2(1H)-one |
| 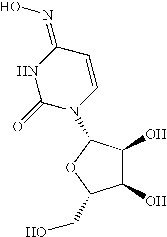 | 4-Oximino-1-(β-L-ribofuranosyl)py- imidin-2(1H)-one |
| 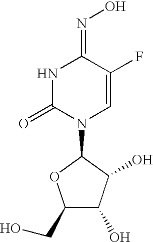 | 5-Fluoro-4-oximino-1-(β-D-ribo- furanosyl)pyrimidin-2(1H)-one |
| 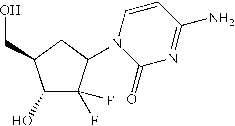 | 4-Amino-1-(2,2-difluoro-3-hydroxy-4-hydroxy- methyl-cyclopentyl)-1H-pyrimidin-2-one |

The novel purine and pyrimidine nucleoside derivatives of formula I have been shown to be inhibitors of subgenomic Hepatitis C Virus replication in a hepatoma cell line. These compounds have the potential to be efficacious as antiviral drugs for the treatment of HCV infections in human. Accordingly, the present novel purine and pyrimidine nucleoside derivatives of formula I are therapeutically active substances in the treatment of HCV infections in human and can be used as medicaments for the treatment of such disease.

The novel purine and pyrimidine nucleoside derivatives of formula I can as well be used as medicaments, especially for treating immune mediated conditions or diseases, viral diseases, bacterial diseases, parasitic diseases, inflammatory diseases, hyperproliferative vascular diseases, tumors, and cancer.

In particular, compounds of the present invention and pharmaceutical compositions containing the same are useful as chemotherapeutic agents, inhibitors of viral replication and modulators of the immune system, and can be used for the treatment of viral diseases such as retroviral infections and hepatitis C virus infections (either alone or in combination with other antiviral agents such as interferon or derivatives thereof, such as conjugates with polyethylene glycol).

They can be used alone, or in combination with other therapeutically active agents, for example, an immunosuppressant, a chemotherapeutic agent, an anti-viral agent, an antibiotic, an anti-parasitic agent, an anti-inflammatory agent, an anti-fungal agent and/or an anti-vascular hyperproliferation agent.

Any functional (i.e. reactive) group present in a side-chain may be protected, with the protecting group being a group which is known per se, for example, as described in “Protective Groups in Organic Synthesis”, 2^{nd} Ed., T. W. Greene and P. G. M. Wuts, John Wiley & Sons, New York, N.Y., 1991. For example, an amino group can be protected by tert.-butyloxycarbonyl (BOC) or benzyloxycarbonyl (Z).

The compounds of this invention may contain one or more asymmetric carbon atoms and may therefore occur as racemates and racemic mixtures, single enantiomers, diastereomeric mixtures and individual diastereomers. Furthermore, where a compound of the invention contains an olefinic double bond, this can have the (E) or (Z) configuration. Also, each chiral center may be of the R or S configuration. All such isomeric forms of these compounds are embraced by the present invention.

Compounds of formula I which are acidic can form pharmaceutically acceptable salts with bases such as alkali metal hydroxides, e.g. sodium hydroxide and potassium hydroxide; alkaline earth metal hydroxides, e.g. calcium hydroxide, barium hydroxide and magnesium hydroxide, and the like; with organic bases e.g. N-ethyl piperidine, dibenzylamine, and the like. Those compounds of formula I which are basic can form pharmaceutically acceptable salts with inorganic acids, e.g. with hydrohalic acids such as hydrochloric acid and hydrobromic acid, sulphuric acid, nitric acid and phosphoric acid, and the like, and with organic acids, e.g. with acetic acid, tartaric acid, succinic acid, fumaric acid, maleic acid, malic acid, salicylic acid, citric acid, methanesulphonic acid and p-toluene sulphonic acid, and the like. The formation and isolation of such salts can be carried out according to methods known in the art.

Also part of the present invention are known purine and pyrimidine nucleoside derivatives for use in medicine, especially for use in the treatment of an Hepatitis C Virus (HCV) infection, where no medical use for those compounds is previously known, and pharmaceutical compositions containing the same.

Assay Method: The activity of the compounds was assayed using an adaptation of the method reported by Lohmann et al [V. Lohmann et al., Science, 1999, 285, 110-113].

HCV Replicon Assay

The HCV replicon-containing cell line is used for the identification of small molecules that are able to inhibit the replication of the replicon RNA. Since the replicon RNA replication mimics the replication of the HCV RNA in infected hepatocytes, it is believed that those small molecules that have the above property are interesting for further development as anti-HCV drugs.

The inhibition of the HCV replicon RNA replication will lead to a decrease of the replicon RNA in the cell, which can be measured using a method that specifically quantifies this RNA.

Northern blot: One method for quantification of this RNA uses the standard Northern blot known to any person skilled in the art.

Kinetic PCR: A second assay for the quantification of replicon RNA is based on the amplification of the replicon RNA that remains in the cell, after incubation of the cells with a proper concentration of the small molecules. This method involves the reverse transcription of the replicon RNA to the corresponding complementary DNA (cDNA), followed by amplification of the cDNA using the Taqman Kinetic PCR technology (PE Biosystems). This consists of hybridisation of the cDNA with a complementary reporter oligonucleotide (probe), containing a combined fluorescent dye and a quencher dye. Amplification of the DNA sequence containing the hybridised reporter probe, using flanking oligonucleotide primers will lead to the separation of the fluorescent dye from the quencher dye. This will result in an increase of the fluorescence during each amplification cycle.

The neomycin phosphotransferase gene sequence that is present in the replicon RNA was chosen for amplification using specifically designed oligonucleotide primers. To control for (a) cell number that can vary depending on the toxicity or cytostatic effect of the small molecules, and (b) for errors during total RNA extraction, amplification of the host β-actin gene is used for normalisation.

The accumulation of the PCR products during the reaction is monitored directly by measuring the increase in fluorescence of the reporter dye. The amount of HCV replicon RNA (and β-actin RNA) originally present in the total RNA extracted from the cells is then expressed as a threshold cycle, e.g. the cycle at which there is a statistically significant increase in the fluorescence above the background.

For this procedure, HCV replicon-containing human hepatoma Huh7 cells (9-13) in growth medium (DMEM) containing 5% FCS are plated in a 96-well plate at 5×10³ cells per well, and the plate incubated overnight. 24 hours later, different dilutions in (0.1 ml growth medium of chemical compounds were added to the wells, and the plate further incubated at 37° C. for three days. Total RNA coming from each well is extracted using the RNeasy™ procedure (Qiagen manufacturer instructions), and the total RNA is eluted in a final volume of 0.13 ml. Next, a 2 μl sample of the total RNA is used for convertion into cDNA using a reverse transcription (RT) step. A RT mastermix containing 1 μl 10×Taqman RT buffer, 2.2 μl 25 mM MgCl₂ (5.5 mM final conc.), 2 μl dNTP mix (500 μM each), 0.5 μl random hexamer primers (2.5 μM), 0.2 μl RNase inhibitor (0.4 u/gl), 0.25 μl RT (1.25 u/μl), 1.85 μl H₂O, was distributed in a 96-well plate and 2 μl total RNA was added to each well.

The RT reaction is performed by incubation of the plate 10 min at 25° C., 30 min at 48° C., 5 min at 95° C. and cooling to 4° C. The cDNA samples are then stored at −20° C. or directly used for the PCR reaction. For the PCR reaction, the cDNA is diluted by addition of 90 μl water, and 10 μl of each diluted cDNA sample is added in duplicate to each well of a 96-well optical plate containing 12.5 μl Taqman Universal PCR mix (PE Biosystems), 1.25 μl 20×Replicon probe/primer mix (Primers 300 nM, Probe 100 nM), 1.25 μl 20×β-actin probe/primer mix (PDAR PE Biosystems). A standard curve is generated for each plate by including in duplicate five 3-fold dilutions of cDNA derived from total RNA extracted from 9-13 cell that were incubated in the absence of chemical compounds. A negative control is included in the plate by omitting the cDNA sample (no template control). Each well of the optical plate is secured with a lid and the plate is mixed. The plate is centrifuged for a few seconds at 3000 rpm to ensure contents are at the bottom of each well. The plate is then inserted into the 7700 Kinetic PCR machine and the reaction started using the default settings.

The concentration of the drug (IC₅₀) required to reduce replicon RNA levels by 50% relative to the untreated 9-13 cell control value, can be calculated from the plot of percentage replicon RNA reduction vs. drug concentration.

Renilla Luciferase reporter: A third assay is based on the idea of using a reporter as a simple readout for intracellular HCV replicon RNA level. For this purpose the Renilla luciferase gene was introduced into the first open reading frame of a replicon construct NK5.1 (Krieger et al., J. Virol. 75:4614), immediately after the internal ribosome entry site (IRES) sequence, and fused with the neomycin phosphotransferase (NPTII) gene via a self-cleavage peptide 2A from foot and mouth disease virus (Ryan & Drew, EMBO Vol 13:928-933). After in vitro transcription the RNA was electroporated into human hepatoma Huh7 cells, and G418-resistant colonies were isolated and expanded. Stably selected cell line 2209-23 was shown to contain replicative HCV subgenomic RNA, and the activity of Renilla luciferase expressed by the replicon reflects its RNA level in the cells.

For the assay procedure, Renilla Luciferase HCV replicon cells (2209-23) that cultured in Dulbecco's MEM (GibcoBRL cat no. 31966-021) with 5% fetal calf serum (FCS) (GibcoBRL cat no. 10106-169) were plated onto a 96-well plate at 5000 cells per well, and incubated overnight. Twenty-four hours later, different dilutions of chemical compounds in the growth medium were added to the cells, which were then further incubated at 37° C. for three days. The assay was carried out in duplicate plates, one in opaque white and one in transparent, in order to measure the activity and cytotoxicity of a chemical compound in parallel ensuring the activity seen is not due to reduction on cell proliferation.

At the end of the incubation time, the cells in the white plate were harvested and luciferase activity was measured by using a Dual-Luciferase reporter assay system (Promega cat no. E1960). All the reagents described in the following paragraph were included in the manufacturer's kit, and the manufacturer's instructions were followed for preparations of the reagents. Briefly, the cells were washed twice with 200 μl PBS (phosphate buffered saline; pH 7.0) per well and lysed with 25 μl of 1×passive lysis buffer prior to incubation at room temperature for 20 min. One hundred microlitre of LAR II reagent was added to each well. The plate was then inserted into the LB 96V microplate luminometer (MicroLumatPlus, Berthold), and 100 μl of Stop & Glo reagent was injected into each well by the machine and the signal measured using a 2-second delay, 10-second measurement programme. The IC₅₀, the concentration of the drug required for reducing the replicon level by 50% in relation to the untreated cell control value, can be calculated from the plot of the percentage reduction of the luciferase activity vs. drug concentration.

Biological Test Results

Compounds were tested for inhibition of HCV replicon RNA replication using the above assay. Examples of the results are shown in the following table:

| Example | Structure | Name | IC₅₀ (μM) |
|---|---|---|---|
| 1 | 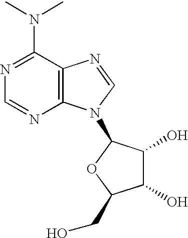 | 6-Dimethylamino-9-(β-D-ribo- furanosyl)purine | 0.6 |
| 7 | 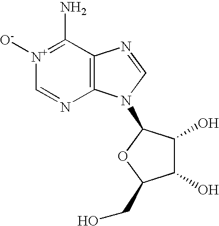 | Adenosine-1-oxide | 2 |
| 10 | 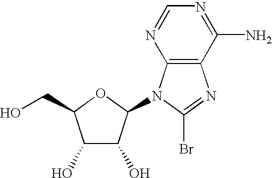 | 8-Bromoadenosine | 3.6 |
| 16 | 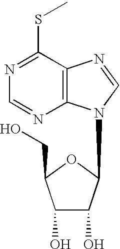 | 6-Methylthio-9-(β-D-ribo- furanosyl)purine | 0.08 |
| 19 | 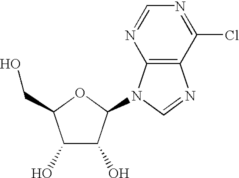 | 6-Chloro-9-(β-D-ribo- furanosyl)purine | 14 |
| 24 | 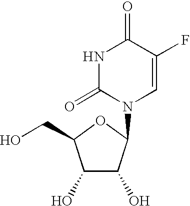 | 5-Fluorouridine | 1.4 |
| 57 | 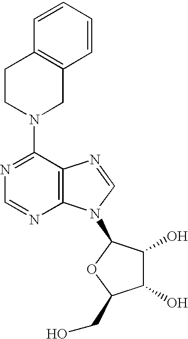 | 9-(β-D-Ribo- furanosyl)-6-(1,2,3,4-tetra- hydro-2-iso- quinolyl)purine | 0.1 |
| 77 | 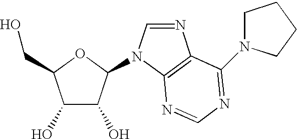 | 6-(1-Pyrrolidinyl)-9-(β-D-ribo- furanosyl)purine | 2.6 |
| 80 | 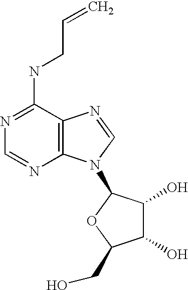 | 6-(2-Propenyl)amino-9-(β-D-ribo- furanosyl)purine | 5.7 |
| 81 | 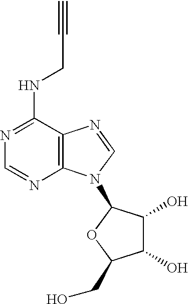 | 6-(2-Propynyl)amino-9-(β-D-ribo- furanosyl)purine | 3.8 |
| 93 | 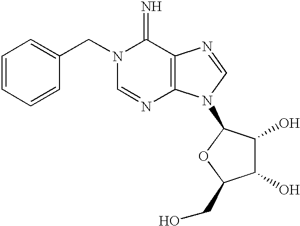 | 1-Benzyl-6-imino-9-(β-D-ribo- furanosyl)purine | 4.5 |
| 105 | 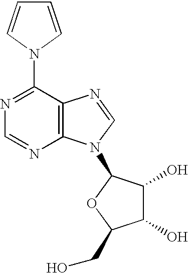 | 6-(1-Pyrrolyl)-9-(β-D-ribo- furanosyl)purine | 0.1 |
| 111 | 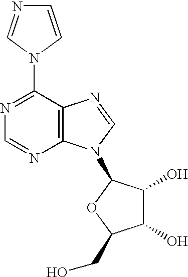 | 6-(1-Imidazolyl)-9-(β-D-ribo- furanosyl)purine | 6.2 |
| 112 | 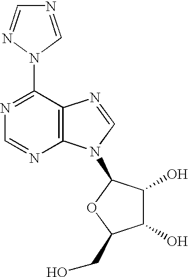 | 9-(β-D-Ribo- furanosyl)-6-1,2,4-tri- azol-1-yl)purine | 4.4 |
| 113 | 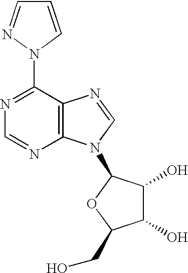 | 6-(1-Pyrazolyl)-9-(β-D-ribo- furanosyl)purine | 4.4 |
| 181 | 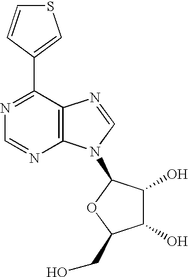 | 9-(β-D-Ribo- furanosyl)-6-(3-thienyl)purine | 0.05 |
| 182 | 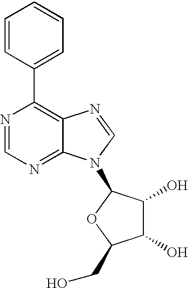 | 6-Phenyl-9-(β-D-ribo- furanosyl)purine | 0.1 |
| 239 | 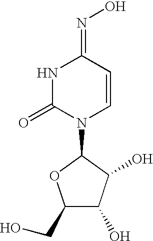 | 4-Oximino-1-(β-D-ribo- furanosyl)pyrimidin- 2(1H)-one | 1.3 |
| 243 | 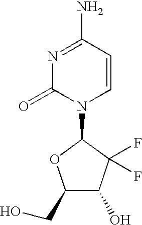 | 1-(2-Deoxy-2,2-difluoro-β-D-ery- thropentofuranosyl)cytosine | 0.07 |
| 244 | 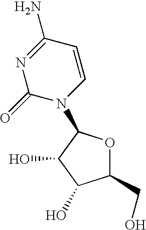 | L-Cytidine | 10 |
| 245 | 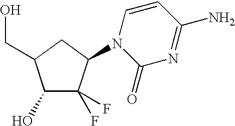 | 4-Amino-1-(2,2-di- fluoro-3-hydroxy-4-hydroxy- methyl-cyclo- pentyl)-1H-py- rimidin-2-one | 2 |
| 246 | 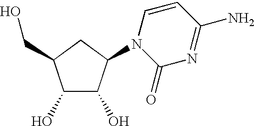 | 4-Amino-1(R)-(2(S),3(R)-di- hydroxy-4(R)-hy- droxymethyl-cyclo- pentyl)-1H-py- rimidin-2-one | 0.4 |
| 247 | 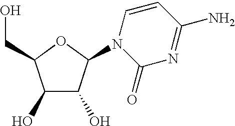 | 1-(β-D-Xylo- furanosyl)cytosine | 3.7 |
| 249 | 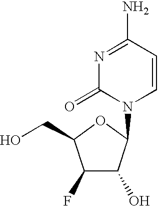 | 1-(3-Deoxy-3-fluoro-β-D-xylo- furanosyl)cytosine | 10.4 |
| 252 | 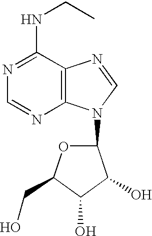 | 6-Ethylamino-9-(β-D-ribo- furanosyl)purine | 14 |
| 253 | 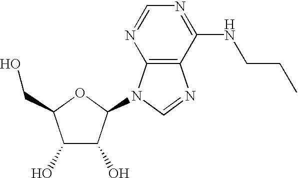 | 6-Propylamino-9-(β-D-ribo- furanosyl)purine | 7 |

Compounds 246, 247, 249, 252 and 253 were tested in the Renilla luciferase assay.

Dosing for the Human Body with Compounds of Formula I

The compounds according to the invention may be employed alone or in combination with other therapeutic agents for the treatment of hepatitis C virus infections.

The compound of formula I whether administered alone or in combination with other therapeutic agents may be administered orally in capsule, tablet or liquid form. Other types of administration could also be contemplated such as nasal spray, transdermally, by suppository, by sustained release dosage form and by pulmonary inhalation, as long as adequate dosages are delivered without destroying the active ingredient.

The amount of the compound of formula I required for the treatment of hepatitis C virus infections will depend on a number of factors including the severity of the disease and the identity, sex and weight of the recipient and will ultimately be at the discretion of the attendant physician. In general, however, a suitable effective dose is in the range of 0.05 to 100 mg per kilogram of body weight of the recipient per day, preferably in the range 0.1 to 50 mg per kilogram of body weight per day and most preferably in the range of 0.5 to 20 mg of body weight per day. An optimum dose is about 2 to 16 mg per kilogram body weight per day. The desired dose is preferably presented as two, three, four, five, six or more sub-doses administered at appropriate intervals throughout the day. These sub-doses may be administered in unit dosage forms, for example, containing from 1 to 1500 mg, preferably from 5 to 1000 mg, most preferably from 10 to 700 mg of active ingredient per unit dosage form.

Combination therapies comprise the administration at least one compound of formula I or a physiologically functional derivative and at least one other physiologically acceptable agent. The active ingredient(s) and physiologically acceptable agent(s) may be administered together or separately and when administered separately this may occur simultaneously or sequentially in any order. The amounts of the active ingredient(s) and physiologically acceptable agent(s) and the relative timings of administration will be selected in order to achieve the desired combined therapeutic effect. Preferably the combination therapy involves the administration of one compound of formula I or a physiologically functional derivative and interferon alpha. The interferon alpha administered is preferably selected from interferon alpha 2a, interferon alpha 2b, a consensus interferon, a purified interferon alpha product or a pegylated interferon alpha 2a or a pegylated interferon alpha 2b. Preferably the amount of interferon alpha administered is from 2 to 10 million IU per week on a weekly, TIW, QOD or daily basis. The preferred method of administering the interferon alpha or pegylated interferon alpha formulations is parenterally, preferably by subcutaneous, IV, or IM injection.

It is preferable to administer the compound of formula I as a pharmaceutical formulation. The formulations of the present invention comprise at least one active ingredient of formula I together with one or more pharmaceutically acceptable exipients and optionally one or more other therapeutic agents. Formulations for oral administration may be capsules, cachets or tablets each containing a predetermined amount of active ingredient(s) may be prepared by any method well known in the art of pharmacy. As well as the active ingredients(s) the oral formulation may contain a binder (for example povidone, gelatin, hydroxypropylmethyl cellulose), a lubricant, inert diluent, preservative, disintegrant (for example sodium starch glycollate, cross-linked povidone, cross-linked sodium carboxymethyl cellulose) or a dispersing agent. Formulations for oral use may also include buffering agents to neutralise stomach acidity.

### EXAMPLE

Tablets containing the following ingredients may be produced in a conventional manner:

| | |
|---|---|
| Ingredient | per tablet |

| | | |
|---|---|---|
| Compound of formula I | 100 | mg |
| Lactose | 131 | mg |
| Microcrystalline cellulose | 60 | mg |
| Croscarmellose sodium | 6 | mg |
| Magnesium stearate | 3 | mg |
| Tablet weight | 300 | mg |

The following examples for the preparation of compounds of formula I illustrate the present invention. The known compounds of formula I are mostly commercially available (the supplier is indicated) or can be synthesised according the below procedure:

Example 1

### 6-Dimethylamino-9-(β-D-ribofuranosyl)purine, Sigma-Aldrich Company Ltd., Cat. No. D2754

### Example 2

### 6-(1(S)-Methyl-2-phenylethylamino)-9-(β-D-ribofuranosyl)purine, Sigma-Aldrich Company Ltd., Cat. No. P7665

### Example 3

### 3′-Deoxyadenosine, Sigma-Aldrich Company Ltd., Cat. No. C3394

### Example 4

### 6-(2-Phenylethylamino)-9-(β-D-ribofuranosyl)purine, Sigma-Aldrich Company Ltd. Cat. No. P2673

### Example 5

### 6-Cyclohexylamino-9-(β-D-ribofuranosyl)purine, Sigma-Aldrich Company Ltd., Cat. No.C9901

### Example 6

### 2-Chloroadenosine, Aldrich Chemical Company, Cat. No. 86,186-3

### Example 7

### Adenosine-1-oxide, Sigma-Aldrich Company Ltd., Cat. No.A8540

### Example 8

### 9-(β-D-Ribofuranosyl)purine, Sigma-Aldrich Company Ltd., Cat. No. P9278

### Example 9

### 3′-Deoxyguanosine, Sigma-Aldrich Company Ltd., Cat. No. D7285

### Example 10

### 8-Bromoadenosine, Aldrich Company Ltd., Cat. No.12,750-7

### Example 11

### 8-Bromo-2′-deoxyadenosine, Maybridge Chemical Company, Cat.

No.BTB14107

### Example 12

### 8-Bromoguanosine, Sigma-Aldrich Company Ltd., Cat. No. B1893

### Example 13

### 6-Thioguanosine, Sigma-Aldrich Company Ltd., Cat. No. M6625

### Example 14

### Inosine, Sigma-Aldrich Company Ltd., Cat. No. I1024

### Example 15

### 6-Thioinosine, Sigma-Aldrich Company Ltd., Cat. No. M7250

### Example 16

### 6-Methylthio-9-(β-D-ribofuranosyl)purine, Sigma-Aldrich Company Ltd., Cat. No. M4002

### Example 17

### L-Inosine, Penta, Cat. No. 09-02700

### Example 18

### 8-Bromoinosine, Sigma-Aldrich Company Ltd., Cat. No. B4004

### Example 19

### 6-Chloro-9-(β-D-ribofuranosyl)purine, Sigma-Aldrich Company Ltd., Cat. No. C8276

### Example 20

### 2-Amino-6-chloro-9-(β-D-ribofuranosyl)purine, Sigma-Aldrich Company Ltd., Cat. No. A4634

### Example 21

### 2′-Deoxy-5-fluorouridine, Sigma-Aldrich Company Ltd., Cat. No. F0503

### Example 22

### 1-(β-D-Arabinofuranosyl)-5-fluorouracil, George-Uhe Company Inc., Cat. No. 000265

### Example 23

### 4-Thiouridine, Sigma-Aldrich Company Ltd., Cat. No. T4509

### Example 24

### 5-Fluorouridine, Sigma-Aldrich Company Ltd., Cat. No. F5130

### Example 25

### 5-Bromouridine, Sigma-Aldrich Company Ltd., Cat. No. B9752

### Example 26

### 3-Methyluridine, Sigma-Aldrich Company Ltd., Cat. No. M4129

### Example 27

### 5-Methyluridine, Sigma-Aldrich Company Ltd., Cat. No. M8905

### Example 28

### 1-(β-D-Arabinofuranosyl)uracil, Sigma-Aldrich Company Ltd., Cat. No. M8905

### Example 29

### 1-(β-D-Arabinofuranosyl)-5-methyluracil, Sigma-Aldrich Company Ltd., Cat. No. T3766

### Example 30

### 1-(β-D-Arabinofuranosyl)-5-iodouracil, George-Uhe Company Inc., Cat. No. 000322

### Example 31

### 3′-Deoxy-5-methyluridine, Berry, Cat. No. PY7260

### Example 32

### 5-Fluorocytidine, ICN Biomedicals Inc., Cat. No. 151156

### Example 33

### 1-(β-D-Arabinofuranosyl)-5-fluorocytosine, Sigma-Aldrich Company Ltd., Cat. No. F3504

### Example 34

### 5-Methylcytidine, Sigma-Aldrich Company Ltd., Cat. No. M4524

### Example 35

### 2′,3′-Dideoxycytidine, Sigma-Aldrich Company Ltd., Cat. No. D5782

### Example 36

### N4-Acetylcytidine, Sigma-Aldrich Company Ltd., Cat. No. A7766

### Example 37

### 3′-Deoxycytidine, Sigma-Aldrich Company Ltd., Cat. No. D5179

0.25g of 6-chloro-9-(β-D-ribofuranosyl)purine and 0.7 g of N-methylpropylamine in 5 ml of anhydrous ethanol were heated at reflux temperature for 1 hour. After cooling to room temperature, the solution was concentrated under reduced pressure and the mixture purified by flash column chromatography on silica gel using methanol/dichloromethane (10:90) as the eluent, to give 0.04 g of 6-(N-methylpropylamino)-9-(β-D-ribofuranosyl)purine as a light yellow solid; mass spectrum (ESI) 324 [M+H]⁺.

### Example 39

Reaction of 6-chloro-9-(β-D-ribofuranosyl)purine with thiomorpholine in an analogous manner to that described in example 38, gave 9-(β-D-ribofuranosyl)-6-(4-thiomorpholinyl)purine as a light brown solid; mass spectrum (ESI) 354 [M+H]⁺.

### Example 40

Reaction of 6-chloro-9-(β-D-ribofuranosyl)purine with N-methylallylamine in an analogous manner to that described in example 38, gave 6-(N-methyl-2-propenylamino)-9-(β-D-ribofuranosyl)purine as an off-white solid; mass spectrum (ESI) 322 [M+H]⁺.

### Example 41

Reaction of 6-chloro-9-(β-D-ribofuranosyl)purine and N-methylpropargylamine in an analogous manner to that described in example 38, gave 6-(N-methyl-2-propynylamino)-9-(β-D-ribofuranosyl)purine as an off-white solid; mass spectrum (ESI) 320 [M+H]⁺.

Also in a manner analogous to that described in example 38 starting with 6-chloro-9-(β-D-ribofuranosyl)purine and the appropriate amine were prepared the following examples:

### Example 42

### 6-(4-Morpholinyl)-9-(β-D-ribofuranosyl)purine, (K. Kikugawa et al, J. Med. Chem., 1972,15, 387)

### Example 43

### 6-Diethylamino-9-(β-D-ribofuranosyl)purine, (Walsh et al, J.Amer.Chem.Soc., 1967, 89, 6221)

### Example 44

### 6-(1(R,S)-Phenylethylamino)-9-(β-D-ribofuranosyl)purine, (S. Kusachi et al, J. Med. Chem., 1985, 28, 1636)

### Example 45

### 6-(1-Benzyl-1-methylethylamino)-9-(β-D-ribofuranosyl)purine, (S. Kusachi et al, J. Med. Chem., 1985, 28, 1636)

### Example 46

### 6-(3-Phenylpropylamino)-9-(β-D-ribofuranosyl)purine, (S. Kusachi et al, J. Med. Chem., 1985, 28, 1636)

### Example 47

### 9-(β-D-Ribofuranosyl)-6-[2-(2-thienyl)ethylamino]purine, (S. Kusachi et al, J. Med. Chem., 1985, 28, 1636)

### Example 48

### 6-Dibenzylamino-9-(β-D-ribofuranosyl)purine, (Endo and Zemlicka, J. Org. Chem., 1979, 44,3652)

### Example 49

### 6-Hexylamino-9-(β-D-ribofuranosyl)purine, (S. Kusachi et al, J. Med. Chem., 1985, 28, 1636)

### Example 50

### 6-(3-Pyridylmethylamino)-9-(β-D-ribofuranosyl)purine, (Kissmann and Weiss, J. Org. Chem., 1956, 21, 1053)

### Example 51

### 6-[4-(4-Fluorophenyl)-1,2,5,6-tetrahydropyridyl]-9-(β-D-ribofuranosyl)purine

### Example 52

### 6-[4-(2-Methoxyphenyl)piperazinyl]-9-(β-D-ribofuranosyl)purine.

### Example 53

### 6-[2-(3-Indolyl)ethylamino]-9-(β-D-ribofuranosyl)purine, (Shikita et al, Chem. Pharm.Bull., 1974, 22,1410)

### Example 54

### 6-[2-(4-Chlorophenyl)ethylamino)]-9-(β-D-ribofuranosyl)purine, (S. Kusachi et al, J. Med. Chem., 1985, 28, 1636)

### Example 55

### 6-(N-Methylphenylamino)-9-(β-D-ribofuranosyl)purine; mass spectrum m/z 358 [M+H]⁺

### Example 56

### 9-(β-D-Ribofuranosyl)-6-(1,2,4,5-tetrahydro-3H-benzazepin-3-yl)purine; mass spectrum m/z 398 [M+H]⁺

### Example 57

### 9-(β-D-Ribofuranosyl)-6-(1,2,3,4-tetrahydro-2-isoquinolyl)purine; mass spectrum m/z 384 [M+H]⁺

### Example 58

### 6-(4-Methylpiperazinyl)-9-(β-D-ribofuranosyl)purine, (H.

Vorbrueggen and K. Krolikiewicz, Liebigs Ann. Chem., 1976, 745)

### Example 59

### 9-(β-D-Ribofuranosyl)-6-(1,3,4,5-tetrahydro-2H-benzazepin-2-yl)purine; mass spectrum m/z 398 [M+H]⁺

### Example 60

### 6-[2-(4-Cyanomethylphenyl)ethylamino]-9-(β-D-ribofuranosyl)purine; mass spectrum m/z 411 [M+H]⁺

### Example 61

### 6-(2,3-Dihydro-1-indolyl)-9-(β-D-ribofuranosyl)purine; mass spectrum m/z 370 [M+H]⁺

### Example 62

### 9-(β-D-Ribofuranosyl)-6-(2,3,4,5-tetrahydro-1,4-benzothiazepin-4-yl)purine; mass spectrum m/z 416 [M+H]⁺

### Example 63

### 9-(β-D-Ribofuranosyl)-6-(2,3,4,5-tetrahydro-1,4-benzoxazepin-4-yl)purine; mass spectrum m/z 400 [M+H]⁺

### Example 64

### 6-(8-Aminosulphonyl-2,3,4,5-tetrahydro-1H-2-benzazepin-2-yl)-9-(β-D-ribofuranosyl)purine; mass spectrum m/z 477 [M+H]⁺

### Example 65

### 6-[2-(3,4-Dimethoxyphenyl)ethylamino)-9-(β-D-ribofuranosyl)purine, (H. Vorbrueggen and K. Krolikiewicz, Liebigs Ann. Chem., 1976, 745)

### Example 66

### 6-[-2-(4-Hydroxyphenyl)ethylamino]-9-(β-D-ribofuranosyl)purine, (Shikita et al, Chem. Pharm.Bull., 1974,22,1410)

### Example 67

### 6-(2-Isoindolinyl)-9-(β-D-ribofuranosyl)purine; mass spectrum m/z 370 [M+H]⁺

### Example 68

### 6-(7-Aminosulphonyl-2,3,4,5-tetrahydro-1H-benzazepin-3-yl)-9-(β-D-Ribofuranosyl)purine; mass spectrum m/z 477 [M+H]⁺

### Example 69

### 6-(N-Cyclohexylmethylamino)-9-(β-D-ribofuranosyl)purine, (Patent No. DE2148838)

### Example 70

### 6-(N-Hexylmethylamino)-9-(β-D-ribofuranosyl)purine, (Patent No. DE2148838)

### Example 71

### 6-(10,11-Dihydro-5H-dibenzo [a,d]cyclohepten-5-ylamino)-9-(β-D-ribofuranosyl)purine; mass spectrum m/z 460 [M+H]⁺

### Example 72

### 6-[N-(10,11-Dihydro-5H-dibenzo[a,d]cyclohepten-5-yl)methylamino]-9-(β-D-ribofuranosyl)purine; mass spectrum m/z 474 [M+H]⁺

### Example 73

### 6-[N-(5-Aminopentyl)methylamino]-9-(β-D-ribofuranosyl)purine; mass spectrum m/z 367 [M+H]⁺

### Example 74

### 6-[(5-Chloro-2-methoxyphenyl)methylamino]-9-(β-D-ribofuranosyl)purine, (Patent No. DE2148838)

### Example 75

### 6-[(2-Methylphenyl)methylamino]-9-(β-D-ribofuranosyl)purine, (A. M. Aronov et al, J. Med. Chem., 1998, 41, 4790)

### Example 76

### 6-(Hexamethyleneimino)-9-(β-D-ribofuranosyl)purine, (H. Vorbrueggen and K. Krolikiewicz, Liebigs Ann. Chem., 1976, 745); mass spectrum (ESI) m/z 350[M+H]⁺

### Example 77

### 6-(1-Pyrrolidinyl)-9-(β-D-ribofuranosyl)purine, (M. Legraverend et al, Tetrahedron, 1984, 40, 709); mass spectrum (ESI) m/z 322 [M+H]⁺

### Example 78

### 6-(4-Hydroxypiperidin-1-yl)-9-(β-D-ribofuranosyl)purine, (Patent No.DE 2157036); mass spectrum (ESI) m/z 352 [M+H]⁺

### Example 79

### 6-(1-Piperidinyl)-9-(β-D-ribofuranosyl)purine, (M. Legraverend et al, Tetrahedron, 1984, 40, 709); mass spectrum (ESI) m/z 336 [M+H]⁺

### Example 80

### 6-(2-Propenyl)amino-9-(β-D-ribofuranosyl)purine, (M. H. Fleysher et al, J. Med. Chem., 1980, 23, 1448); mass spectrum (ESI) m/z 308 [M+H]⁺

### Example 81

### 6-(2-Propynyl)amino-9-(β-D-ribofuranosyl)purine, (M. H. Fleysher et al, J. Med. Chem., 1980, 23, 1448); mass spectrum (ESI) m/z 306 [M+H]⁺

### Example 82

### 6-(1-Methyl)ethylamino-9-(β-D-ribofuranosyl)purine, (A. M. Aronov et al, J. Med. Chem., 1998, 41, 4790) mass spectrum (ESI) m/z 310 [M+H]⁺

### Example 83

### 6-bis-(2-Propenyl)amino-9-(β-D-ribofuranosyl)purine, (Patent No. DE 2338963); mass spectrum (ESI) m/z 348 [M+H]⁺

### Example 84

### 6-(2-Phenylethyl)methylamino-9-(β-D-ribofuranosyl)purine ,(S. Kusachi et al, J. Med. Chem., 1985, 28, 1636); mass spectrum (ESI) m/z 386 [M+H]⁺

### Example 85

### 6-Ethylmethylamino-9-(β-D-ribofuranosyl)purine; mass spectrum (ESI) m/z 310 [M+H]⁺

### Example 86

### 6-bis-[(3-Methyl)butylamino]-9-(β-D-ribofuranosyl)purine; mass spectrum (ESI) m/z 408 [M+H]⁺

### Example 87

### 6-(4-Aminophenyl)methylamino-9-(β-D-ribofuranosyl)purine, (M. J. Robins et al, Nucleosides and Nucleotides, 1994, 13, 1627)

### Example 88

### 6-(2-Pyridylmethyl)amino-9-(β-D-ribofuranosyl)purine ,(S. Kusachi et al, J. Med. Chem., 1985,28, 1636); mass spectrum (ESI) m/z 359 [M+H]⁺

### Example 89

### 6-(2-Hydroxyethyl)methylamino-9-(β-D-ribofuranosyl)purine (P. F. Guengerich and V. M. Raney, J.Amer.Chem.Soc., 1992,114,1074)

### Example 90

### 6-Dipropylamino-9-(β-D-ribofuranosyl)purine, (M. de Zwart et al, Nucleosides and Nucleotides, 1998, 17, 969)

### Example 91

Starting with 2′,3′,5′-tris-O-(tert-butyldimethylsilyl)adenosine in manner analogous to that described by K. Aritomo, T. Wada and M. Sekine, J. Chem. Soc. Perkin Trans.1, 1995,1837 was prepared 6-[2-phenyl-(N-propionyl)ethylamine)-9-(β-D-ribofuranosyl)purine; mass spectrum m/z 428 [M+H]⁺.

### Example 92

Starting with 2′,3′,5′-tris-O-(tert-butyldimethylsilyl)adenosine in manner analogous to that described by K. Aritomo, T. Wada and M. Sekine, J. Chem. Soc. Perkin Trans. 1, 1995,1837 was prepared 6-(N-benzoyl-2-phenylethylamine)-9-(β-D-ribofuranosyl)purine; mass spectrum m/z 476 [M+H]⁺.

### Example 93

Starting with adenosine in manner analogous to that described by T. Itaya et al, Chem. Pharm. Bull., 1977, 25, 1449 was prepared 1-benzyl-6-imino-9-(β-D-ribofuranosyl)purine.

### Example 94

Starting with 6-(2-phenylethylamino)-9-(β-D-ribofuranosyl)purine (prepared in a manner analogous to that described in example 83) and in manner analogous to that described by T. Itaya et al, Chem. Pharm. Bull., 1977, 25, 1449 was prepared 1-methyl-6-(2-phenylethylamino) -9-(β-D -ribofuranosyl)purine; mass spectrum m/z 386 [M+H]⁺.

### Example 95

A solution of 0.34 g of 2-amino-6-chloro-9-(2,3,5-tri-O-benzoyl-β-L-ribofuranosyl)purine in 5 ml of a 2M solution of methylamine in methanol was heated under nitrogen under reflux overnight. The solvents were removed by evaporation and the residue purified by preparative HPLC to give 10 mg of 2-amino-6-methylamino-9-(β-L-ribofuranosyl)purine as a pale yellow solid; mass spectrum (ESI) m/z 297[M+H]⁺.

The 2-amino-6-chloro-9-(2,3,5-tri-O-benzoyl-β-L-ribofuranosyl)purine used as the starting material was prepared as follows:

A suspension of 38 mg of 2-amino-6-chloropurine in 1 ml of anhydrous acetonitrile was treated with 0.22 ml of bis(trimethylsilyl)acetamide and heated at reflux for 15 min. To the resulting solution was added a solution of 95 mg of 1-O-acetyl-2,3,5-tri-O-benzoyl-L-ribose in 1 ml of anhydrous acetonitrile followed by 51 μl of trimethylsilyl trifluoromethanesulphonate. The solution was heated at reflux under nitrogen for 2.5 hours. After cooling to room temperature the solution was evaporated and the residue dissolved in dichloromethane and washed twice with water. The solution was dried over anhydrous magnesium sulphate, filtered and evaporated to give crude 2-amino-6-chloro-9-(2,3,5-tri-O-benzoyl-β-L-ribofuranosyl)purine which was used without further purification; mass spectrum (ESI) m/z 614 [M+H]⁺.

### Example 96

Reaction of 2-amino-6-chloropurine with 1-O-acetyl-2,3,5-tri-O-benzoyl-D-ribose followed by treatment of the intermediate 2-amino-6-chloro-9-(2,3,5-tri-O-benzoyl-β-D-ribofuranosyl)purine with methylamine in methanol in an analogous manner to that described in example 95 gave 2-amino-6-methylamino-9-(β-D-ribofuranosyl)purine (R. Saladino et al, Tetrahedron, 1996, 52, 6759); mass spectrum (ESI) m/z 297[M+H]⁺.

### Example 97

Reaction of 2-amino-6-chloropurine with 1-O-acetyl-2,3,5-tri-O-benzoyl-D-ribose followed by treatment of the intermediate 2-amino-6-chloro-9-(2,3,5-tri-O-benzoyl-β-D-ribofuranosyl)purine with morpholine in methanol in an analogous manner to that described in example 95 gave 2-amino-6-(4-morpholinyl)-9-(β-D-ribofuranosyl)purine (H. Vorbrueggen and K. Krolikiewicz, Justus Leibigs Ann.Chem.,1976, 745).

### Example 98

Reaction of 2-amino-6-chloropurine with 1-O-acetyl-2,3,5-tri-O-benzoyl-D-ribose followed by treatment of the intermediate 2-amino-6-chloro-9-(2,3,5-tri-O-benzoyl-β-D-ribofuranosyl)purine with pyrrolidine in methanol in an analogous manner to that described in example 95 gave 2-amino-6-(1-pyrrolidinyl)-9-(β-D-ribofuranosyl)purine; mass spectrum (ESI) m/z 337 [M+H]⁺.

### Example 99

A suspension of 84 mg of 2,4-diaminopurine in 2 ml of anhydrous acetonitrile was treated with 0.55 ml of bis(trimethylsilyl)acetamide and the solution heated at reflux for 15 min to give a solution. To the solution was added a solution of 237 mg of 1-O-acetyl-2,3,5-tri-O-benzoyl-L-ribose in 2 ml of anhydrous acetonitrile. The solution was heated at reflux under nitrogen for 16 hours. After cooling to room temperature the solution was evaporated and the residue dissolved in dichloromethane and washed with water. The dichloromethane solution was dried over anhydrous magnesium sulphate, filtered and evaporated. The residue was dissolved in 10 ml of a 2M solution of ammonia in methanol and the solution stirred at room temperature for 42 hours then evaporated. The residue was purified by preparative HPLC to give 50 mg of 2,6-diamino-9-(β-L-ribofuranosyl)purine, (D. M. Brown et al, Nucleosides and Nucleotides, 1999, 18, 2521); mass spectrum (ESI) m/z 283[M+H]⁺.

### Example 100

Reaction of 2,6-diaminopurine with 1-O-acetyl-2,3,5-tri-O-benzoyl-D-ribose followed by treatment of the intermediate 2,6-diamino-9-(2,3,5-tri-O-benzoyl-β-D-ribofuranosyl)purine with ammonia in methanol in an analogous manner to that described in example 99 gave 2,6-diamino-9-(β-D-ribofuranosyl)purine (also available commercially from ICN Biomedicals Inc.).

### Example 101

A mixture of 4.5 g of 2,6-dichloro-9-(2,3,5-tri-O-acetyl-β-D-ribofuranosyl)purine, 1.1 g of pyrrolidine and 2.8 ml of triethylamine in 50 ml of benzene was stood at room temperature for 1 hour then washed with water, dried and evaporated. The residue was dissolved in a saturated solution of ammonia in methanol and the solution stood overnight at room temperature. The solution was evaporated and the residue recrystallised from n-butanol to give 2.5 g of 2-chloro-6-(1-pyrrolidinyl)-9-(β-D-ribofuranosyl)purine (W. Kampe et al, Patent No. DE 2157036) of melting point 229° C.; mass spectrum (ESI) m/z 356 [M+H]⁺.

### Example 102

By an analogous procedure to that described in example 101 starting with 2,6-dichloro-9-(2,3,5-tri-O-acetyl-β-D-ribofuranosyl)purine and hexamethyleneimine was prepared 2-chloro-6-(1-hexamethyleneimino)-9-(β-D-ribofuranosyl)purine, (W. Kampe et al, Patent No. DE 2157036); mass spectrum (ESI) m/z 384 [M+H]⁺.

### Example 103

By an analogous procedure to that described in example 101 starting with 2,6-dichloro-9-(2,3,5-tri-O-acetyl-β-D-ribofuranosyl)purine and 4-hydroxypiperidine was prepared 2-chloro-6-(4-hydroxy-1-piperidinyl) -9-(β-D-ribofuranosyl)purine (W. Kampe et al, Patent No. DE 2157036); mass spectrum (ESI) m/z 386 [M+H]⁺.

### Example 104

By a procedure analogous to that described by Kissman et al, J. Amer. Chem. Soc., 1955, 77,18 was prepared 6-[(N-cyclohexyl)methylamino]-2-methylthio-9-(β-D-ribofuranosyl)purine; mass spectrum (ESI) mlz 410 [M+H]⁺.

### Example 105

A solution of 30 g of adenosine and 16.4 ml of 2,5-dimethoxytetrahydrofuran in 70 ml of glacial acetic acid was heated at reflux temperature for 1 hour. After cooling to room temperature the mixture was concentrated under reduced pressure, and the residual oil triturated with acetone, filtered and the filtrate evaporated. The residue was purified by column chromatography on silica gel using methanol/dichloromethane (5:95) as the eluent to give 17.0 g of 6-(1-pyrrolyl)-9-(β-D-ribofuranosyl)purine as a light orange solid; mass spectrum (ESI) m/z 318 [M+H].

### Example 106

Reaction of 6-amino-9-(β-D-arabinofuranosyl)purine with dimethoxytetrahydrofuran in an analogous manner to that described in example 105 gave 6-(1-pyrrolyl)-9-(β-D-arabinofuranosyl)purine as a light brown solid of melting point 212-213° C.; mass spectrum (ESI) 318 [M+H]⁺.

### Example 107

A solution containing 150 mg of 6-amino-9-(β-D-ribofuranosyl)purin-8-(7H)-one and 74 mg of 2,5-dimethoxytetrahydrofuran in 5 ml glacial acetic acid was heated under nitrogen at 110° C. for 1 hour. The solvents were then evaporated under low vacuum to give a brown residue, which was purified by flash chromatography on silica-gel using methanol/dichloromethane (1:9) for the elution to give 18 mg of 6-(1-pyrrolyl)-9-(β-D-ribofuranosyl)purin-8(7H)-one as a white solid; mass spectrum (ESI) m/z 334 [M+H]⁺.

### Example 108

A solution containing 150 mg of 9-(3′-deoxy-β-D-ribofuranosyl)adenosine and 83 mg of 2,5-dimethoxytetrahydrofuran in 5 ml glacial acetic acid was heated under nitrogen at 110° C. for 2 hours. The solvents were then evaporated under low vacuum to give a beige solid which was purified by flash chromatography on silica-gel using methanol/dichloromethane (1:49) for the elution to give 70 mg of 9-(3-deoxy-β-D-ribofuranosyl)-6-(1-pyrrolyl) purine as a white solid of melting point 175-176° C.; mass spectrum (ESI) m/z 302 [M+H]⁺.

### Example 109

A solution of 0.51 g of 6-(1-pyrrolyl)-9-(2,3,5-tri-O-benzoyl-β-L-ribofuranosyl)purine and 20 ml of a 33% aqueous ammonia solution in 30 ml of methanol/tetrahydrofuran (1:1), was heated at 50° C. for 2 hours. After cooling to room temperature the mixture was evaporated, diluted with 50 ml of water and extracted twice with 50 ml diethyl ether followed by 50 ml ethyl acetate. The combined organic extracts were dried over anhydrous sodium sulphate, concentrated under reduced pressure and the mixture purified by column chromatography on silica gel using methanol/dichloromethane (5:95) as the eluent, to give 0.12 g of 6-(1-pyrrolyl)-9-(β-L-ribofuranosyl)purine as a white solid of melting point 114-115° C.; mass spectrum (ESI) 318 [M+H]⁺.

The 6-(1-pyrrolyl)-9-(2,3,5-tri-O-benzoyl-β-L-ribofuranosyl)purine used as a starting material was prepared as follows:

To a suspension of 1.0 g of 6-(1-pyrrolyl)purine (prepared according to K. G. Estep et al, J.Med.Chem., 1995, 38, 2582) and 0.97 g of 1-O-acetyl-2,3,5-tri-O-benzoyl-β-L-ribofuranose in 30 ml of 1,2-dichloroethane was added dropwise 2.30 g of N-methyl-N-trimethylsilyl trifluoroacetamide, and the mixture heated to 80° C. Following addition of 0.635 g of trimethylsilyl trifluoromethane sulphonate dropwise, the mixture was stirred at 80° C. overnight. After cooling to room temperature, the mixture was diluted with 60 ml of dichloromethane and washed four times with a saturated solution of aqueous sodium hydrogen carbonate. The organic extract was dried over sodium sulphate, filtered and evaporated and the residue purified by flash column chromatography on silica gel using ethyl acetate/hexane (10:90) for the elution to give 0.56 g of 6-(1-pyrrolyl)-9-(2,3,5-tri-O-benzoyl-β-L-ribofuranosyl)purine as a white solid; mass spectrum (ESI) 630 [M+H].

### Example 110

Reaction of 6-(1-indolyl)purine (M. Haidoune and R Mornet, J. Hetercyclic Chem., 1994, 31, 1461) with 1-O-acetyl-2,3,5-tri-O-benzoyl-β-D-ribofuranose followed deprotection in an analogous manner to that described in example 109 gave 6-(1-indolyl)-9-(β-D-ribofuranosyl)purine; mass spectrum m/z 368 [M+H]⁺.

### Example 111

Reaction of 6-(1-imidazol-yl)purine (G. E. Estep et al, J. Med. Chem., 1995, 38, 2582)) with 1-O-acetyl-2,3,5-tri-O-benzoyl-β-D-ribofuranose followed deprotection in an analogous manner to that described in example 109 gave 6-(1-imidazolyl)-9-(β-D-ribofuranosyl)purine; mass spectrum m/z 319 [M+H]⁺.

### Example 112

150 μl of a 1M solution of sodium methoxide in methanol was added to a stirring solution of 0.445 g of 6-(1,2,4-triazol-1-yl)-9-(2,3,5-tri-O-acetyl-β-D-ribofuranosyl)purine in 10 ml of anhydrous methanol. After stirring overnight at room temperature a few drops of glacial acetic acid were added and the mixture concentrated under reduced pressure. The mixture was purified by column chromatography on silica gel using an eluent of methanol/dichloromethane (10:90) to give 0.2 g of 9-(β-D-ribofuranosyl)-6-(1,2,4-triazol-1-yl)purine as a white solid, mass spectrum (ESI) 320 [M+H].

The 6-(1,2,4-triazol-1-yl)-9-(2,3,5-tri-O-acetyl-β-D-ribofuranosyl)purine used as a starting material was prepared as follows:

3.7 ml of Phosphorous oxychloride followed by 30 ml of triethylamine were added dropwise to a solution of 13.1 g of 1,2,4-triazole in 150 ml of acetonitrile at <5° C. After stirring for 1 hour, a suspension of 5.0 g of 2′,3′,5′-tri-O-acetylinosine in 150 ml of acetonitrile was added, and the mixture stirred at room temperature overnight. The mixture was filtered, diluted with 100 ml of ethyl acetate and extracted twice with 100 ml of a saturated solution of aqueous sodium hydrogen carbonate. The organic extract was dried over anhydrous sodium sulphate and concentrated under reduced pressure. The mixture was purified by column chromatography on silica gel using methanol/dichloromethane (5:95) for the elution to give 2.7 g of 6-(1,2,4-triazol-1-yl)-9-(2,3,5-tri-O-acetyl-β-D-ribofuranosyl)purine as a white foam, mass spectrum (ESI) 446 [M+H].

### Example 113

Reaction of 6-(1-pyrazolyl)-9-(2,3,5-tri-O-benzoyl-β-D-ribofuranosyl)purine with sodium methoxide in an analogous manner to that described in example 112 followed by purification by supercritical fluid chromatography gave 6-(1-pyrazolyl)-9-(β-D-ribofuranosyl)purine as a white solid, mass spectrum (ESI) 319 [M+H].

The 6-(1-pyrazolyl)-9-(2,3,5-tri-O-benzoyl-β-D-ribofuranosyl)purine used as a starting material was prepared as follows:

0.78 ml of chlorotrimethylsilane was added dropwise to a stirring solution of 0.372 g of 6-(1-pyrazolyl)purine (prepared according to K. G. Estep et al, J. Med.Chem., 1995, 38, 2582) 1.0 g of β-D-ribofuranose-1-acetate-2,3,5-tribenzoate, 1.62 g of nonafluoro-1-butanesulfonic acid and 0.3 ml of hexamethyldisilazane in 30 ml of acetonitrile, and the mixture heated at reflux temperature for 21 hours. After cooling to room temperature, the mixture was diluted with 30 ml of dichloromethane and washed with 50 ml of a saturated aqueous solution of sodium hydrogen carbonate. The organic extract was dried over anhydrous sodium sulphate and concentrated under reduced pressure. The mixture was purified by column chromatography on silica gel using an eluent of methanol/dichloromethane (5:95) to give 0.06 g of 6-(pyrazol-1-yl)-9-(2,3,5-tri-O-benzoyl-β-D-ribofuranosyl)purine as a yellow solid, mass spectrum (ESI) 630 [M+H].

### Example 114

By the procedure of V. Samano, R. W. Robins and M. J. Robins, J. Amer. Chem. Soc., 1994, 116, 9331 was prepared 9-(β-D-ribofuranosyl) 6-(1,2,4-triazol-4-yl)purine; mass spectrum (ESI) m/z 320[M+H]⁺.

### Example 115

By a procedure analogous to that of J. A. Montogomery, J. A. Secrist and C. A. Krauth, U.S. Pat. No. 5,102,873 starting with adenosine was prepared 6-(2-phenylethylamino)-9-(β-D-ribofuranosyl)purine-1-oxide.

### Example 116

By the procedure of Yamazaki et al, Chem. Pharm. Bull., 1968, 16, 2172 was prepared 6-methylamino-9-(β-D-ribofuranosyl)purin-2(1H)-one of melting point 270° C. (decomposition).

### Example 117

By the procedure of G. R. Gough and H. M. Maguire, J.Med.Chem., 1967,10, 475 was prepared 2-methoxy-6-methylamino-9-(1-β-D-ribofuranosyl)purine of melting point 142° C. (decomposition).

### Example 118

By the procedure of T. Schaeffer, J. Amer. Chem. Soc., 1958, 80,3738 starting with 2-chloroadenosine (Aldrich Chemical Co.) was prepared 2-methoxyadenosine.

### Example 119

By the procedure of J. F. Gerster and R. K. Robins, J. Org. Chem., 1966, 31, 3528 was prepared 2-amino-6-chloro-9-(β-D-ribofuranosyl)purine.(Sigma-Aldrich Chemical Co.).

### Example 120

By the procedure of Johnson et al, J.Amer.Chem.Soc., 1958, 80; 699 starting with 6-chloro-9-(β-D-ribofuranosyl)purine was prepared 6-methoxy-9-(1-β-D-ribofuranosyl)purine; mass spectrum (ESI) m/z 283 [M+H]⁺.

### Example 121

By the procedure of C. W. Noell and R. K. Robins, J. Med. Pharm. Chem., 1962, 5, 1074 was prepared 2-amino-6-benzylthio-9-(β-D-ribofuranosyl)purine.

### Example 122

By the procedure of W. Kampe et al, Patent No. ZA 6707630 was prepared 6-benzylthio-2-hydroxy-9-(β-D-ribofuranosyl)purine; mass spectrum (ESI) m/z 391 [M+H]⁺.

### Example 123

By the procedure of B. S. Schultz and W. Pleiderer, Tet. Lett., 1985, 26, 5421 from guanosine was prepared 9-(β-D-ribofuranosyl)purine-2,6,8(1H,3H,7H)-trione; mass spectrum (ESI) m/z 342[M+CH₃CN+H]⁺.

### Example 124

By the procedure of C. B. Reese and R. Saffhill, J. Chem. Soc. Perkin Trans. 1, 1972, 2937 was prepared 2-(acetylamino)inosine; mass spectrum (ESI) m/z 326[M+H]⁺.

### Example 125

A mixture of 0.5 g of 8-bromoadenosine and 0.5 ml of water was treated with 1 ml of a 33% solution of methylamine in ethanol. The mixture was heated at 70° C. for 12 hours then evaporated to dryness . The crude product (0.54 g) was purified by flash column chromatography on silica gel using methanol/dichloromethane (1:9 to 3:9) for the elution to give 0.34 g of 8-(methylamino)adenosine (J. B. Chattopadhyaya and C. B. Reese, Synthesis, 1977, 725) as a white solid of melting point >250° C.; mass spectrum (ESI) m/z 297 [M+H]⁺.

In a manner analogous to that described in example 125 starting with 8-bromoadenosine and the appropriate amine in ethanol or aqueous ethanol were prepared the following examples:

### Example 126

### 8-(2-Phenylethylamino)adenosine

### Example 127

### 8-Benzylaminoadenosine (A. M. Aronov and M. H. Gelb, Biorg.and Med.Chem.Lett., 1998,24,3505) of melting point 213-216° C.

### Example 128

### 8-(1-Piperidinyl)adenosine (A. M. Aronov and M. H. Gelb, Biorg.and Med.Chem.Lett. 1998,24,3505) of melting point 207-209° C. (decomposition)

### Example 129

### 8-(Dimethylamino)adenosine (A. M. Aronov and M. H. Gelb, Biorg.and Med.Chem.Lett. 1998,24,3505) of melting point 205-207° C.

### Example 130

### 8-(3-Phenylpropylamino)adenosine of melting point 180-183° C.

### Example 131

### 8-(4-Morpholinyl)adenosine of melting point 210-213° C.

### Example 132

### 8-(N-Methyl-2-phenylethylamino)adenosine of melting point 118-120° C.

### Example 133

### 8-(3-Pyridylmethylamino)adenosine of melting point 235-237° C. (decomposition)

### Example 134

### 8-(Ethylamino)adenosine (R. A. Long and R. K. Robins, J.Org.Chem., 1967, 32, 2751) of melting point 260-170° C.

### Example 135

### 8-(1,2,3,4-Tetrahydro-2-isoquinolyl)adenosine of melting point 145-150° C. (decomposition)

### Example 136

### 8-[2-(4-Morpholinyl)ethylamino]adenosine of melting point 210-215° C.

### Example 137

### 8-(Hexylamino)adenosine (Patent No. JP53124293) of melting point 209-212° C.

### Example 138

### 8-(2-Cyclohexylethylamino)adenosine of melting point 203-205° C.

### Example 139

### 8-(2(R,S)-Phenylpropylamino)adenosine of melting point 159-161° C. (decomposition)

### Example 140

### 8-[2-(4-Methylphenyl) ethylamino]adenosine of melting point 117-124° C. (decomposition)

### Example 141

### 8-[2-(1-Methyl-2-pyrrolyl) ethylamino]adenosine of melting point 225-228° C.

### Example 142

### 8-[2-(4-Aminosulphonylphenyl) ethylamino]adenosine of melting point 157-163° C. (decomposition)

### Example 143

### 8-(4-Phenyl-1-piperazinyl)adenosine of melting point 220-223° C. (decomposition)

### Example 144

### 8-(2-(4-Imidazolyl)adenosine (T. Prakash and K. N. Ganesh, J.Chem.Soc.Chem.Commun.,1994,1357) of melting point 148-156° C. (decomposition)

### Example 145

### 8-(1-Naphthylmethylamino)adenosine of melting point 140-150° C.

### Example 146: 8-[2-(4-Hydroxyphenyl)ethylamino]adenosine of melting point 262-265° C. (decomposition)

### Example 147

### 8-(4-Phenylbutylamino)adenosine of melting point 190° C.

### Example 148

### 8-[2-(4-Chlorophenyl)ethylamino]adenosine of melting point 155-158° C. (decomposition)

### Example 149

### 8-[2-(2,4-Dichlorophenyl)ethylamino]adenosine of melting point 164-168° C. (decomposition)

### Example 150

### 8-(2-Propenylamino)adenosine of melting point 234-237° C. (decomposition)

### Example 163

### 8-[(4-tert-Butyl)benzylamino]adenosine of melting point 187-190° C.

### Example 164

### 8-(1(R)-Phenylethylamino)adenosine of melting point 120-130° C.

### Example 165

### 8-(1(S)-Phenylethylamino)adenosine of melting point 112-130° C.

### Example 166

### 8-(6-Phenylhexylamino)adenosine of melting point 165-167° C.

### Example 167

### 8-[2-Hydroxy-1(S)-phenyl)ethylamino]adenosine of melting point 110-125° C.

By a procedure analogous to that described in example 125 from 8-bromo-2′-deoxyadenosine were prepared the following examples:

### Example 168

### 2′-Deoxy-8-(2-phenylethylamino)adenosine of melting point 192-195° C.

### Example 169

### 2′-Deoxy-8-(3-phenylpropylamino)adenosine of melting point 198-201° C.

### Example 170

### 8-Benzylamino-2′-deoxyadenosine of melting point 132-134° C.

### Example 171

### 2′-Deoxy-8-(4-phenylbutylamino)adenosine of melting point 168-171° C.

### Example 172

### 2′-Deoxy-8-(6-phenylhexylamino)adenosine of melting point 159-161° C.

### Example 173

By a procedure analogous to that described in example 125 from 8-bromoinosine was prepared 8-(4-morpholinyl)inosine (M. Sechenova, Fiziol.Zh.SSSR, 1989, 75, 457).

### Example 174

By a procedure analogous to that described in example 125 from 8-bromoinosine was prepared 8-benzylaminoinosine (Chattopaohyaya and Reese, Synthesis,1978, 908) of melting point 225-228° C.

### Example 175

By the procedure of G. S. Buenger, Synthesis, 1990,962 starting with 8-bromoadenosine was prepared 8-(methylthio)adenosine of melting point 254-255° C.

### Example 176

By an analogous procedure to that of G. S. Buenger, Synthesis, 1990,962 starting with 8-bromoadenosine was prepared 8-(benzylthio)adenosine (E, Liepins et al, Bioorg. Khim., 1988,14,1393) of melting point 206-210° C.

### Example 177

By the procedure of G. S. Buenger, Synthesis, 1990, 962 starting with 8-bromoadenosine was prepared 8-(benzyloxy)adenosine of melting point 199-201° C.

### Example 178

By an analogous procedure to that of G. S. Buenger, Synthesis, 1990,962 starting with 8-bromoadenosine was prepared 8-ethoxyadenosine of melting point 172-175° C.

### Example 179

By the procedure of Holmes and Robins, J. Amer. Chem. Soc., 1964, 86, 1242 starting with 8-bromoadenosine was prepared 6-amino-9-(β-D-ribofuranosyl)purine-8(7H)-thione of melting point 242-248° C. (decomposition).

### Example 180

By the procedure of H. Steinmaus et al, J. Org.Chem., 1971, 36,3594 starting with adenosine was prepared 8-[(1-hydroxy-1-methyl)ethyl]adenosine.

### Example 181

A solution of 0.31 g of 9-(2,3,5-tri-O-acetyl-β-D-ribofuranosyl)-6-(3-thienyl)purine in 3 ml of anhydrous methanol was treated with 67 μl of a 1M solution of sodium methoxide in methanol. The mixture was stirred at room temperature for 2 hours during which time a white precipitate separated. A few drops of glacial acetic acid were added and the mixture was evaporated to dryness under reduced pressure. Recrystallisation of the residue from ethanol gave 0.11 g of 9-(β-D-ribofuranosyl)-6-(3-thienyl)purine as a white solid of melting point 166-167° C. (decomposition); mass spectrum (ESI) m/z 335[M+H]⁺.

The 9-(2,3,5-tri-O-acetyl-β-D-ribofuranosyl)-6-(3-thienyl)purine used as the starting material was prepared as follows:

A mixture containing 0.5 g of 9-(2,3,5-tri-O-acetyl-β-D-ribofuranosyl)-6-chloropurine, 0.23 g of thiophene-3-boronic acid, 0.21 g of anhydrous potassium carbonate and 0.034 g of tetrakis-(triphenylphosphine)palladium in 24 ml of anhydrous toluene was stirred under nitrogen and heated at 100° C. for 5 hours. After cooling the mixture was diluted with 50 ml of ethyl acetate and washed with 20 ml of water and 20 ml of brine. The solution was dried over anhydrous magnesium sulphate, filtered and evaporated to yield a gum. This was purified by flash chromatography on silica gel using ethyl acetate/hexane(1:1) for the elution to give 0.31 g of 9-(2,3,5-tri-O-acetyl-β-D-ribofuranosyl)-6-(3-thienyl)purine as a gum; mass spectrum (ESI) m/z 461[M+H]⁺.

### Example 182

Reaction of 6-chloro-9-(2,3,5-tri-O-acetyl-β-D-ribofuranosyl)purine with phenylboronic acid followed by deprotection in an analogous manner to that described in example 181 gave 6-phenyl-9-(β-D-ribofuranosyl) purine, (M.

Hoceck, A. Holy, I. Votruba and H. Dvorakova, J. Med. Chem., 2000, 43, 1817) as a white solid of melting point 224-225° C.; mass spectrum (ESI) m/z 329[M+H]⁺.

Reaction of 50 mg samples of 6-chloro-9-(tri-O-acetyl-β-D-ribofuranosyl)purine with a range of arylboronic acids in an analogous manner to that described in example 181 was carried out in parallel using a Mettler Toledo Myriad reactor. The intermediate crude 6-aryl-9-(2,3,5-tri-O-acetyl-β-D-ribofuranosyl)purines were purified using a Jones Flashmaster II sequential chromatography system using ethyl acetate/hexane for the elution before deprotection using sodium methoxide in methanol in an analogous manner to that described in example 181 to give the 6-aryl-9-(β-D-ribofuranosyl)purines listed below:

### Example 183

### 6-(4-Fluorophenyl)-9-(β-D-ribofuranosyl)purine (M Hocek et al, J Med Chem, 2000, 43, 1817); mass spectrum (ESI) m/z 347[M+H]⁺

### Example 184

### 6-(4-Chlorophenyl)-9-(β-D-ribofuranosyl)purine (M Hocek et al, J Med Chem, 2000, 43, 1817); mass spectrum (ESI) m/z 363[M+H]⁺

### Example 185

### 6-(4-Methylphenyl)-9-(β-D-ribofuranosyl)purine (M Hocek et al, J Med Chem, 2000, 43, 1817); mass spectrum (ESI) m/z 343[M+H]⁺

### Example 186

### 6-(4-Methoxyphenyl)-9-(β-D-ribofuranosyl)purine(M Hocek et al, J Med Chem, 2000, 43, 1817); mass spectrum (ESI) m/z 359[M+H]⁺

### Example 187

### 9-(β-D-Ribofuranosyl)-6-(1-thianthrenyl)purine; mass spectrum (ESI) m/z 467[M+H]⁺

### Example 188

### 6-(4-Biphenylyl)-9-(β-D-ribofuranosyl)purine; mass spectrum (ESI) m/z 405[M+H]⁺

### Example 189

### 6-(4-Methylthiophenyl)-9-(β-D-ribofuranosyl)purine; mass spectrum (ESI) m/z 375[M+H]⁺

### Example 190

### 6-(2-Methylphenyl)-9-(β-D-ribofuranosyl)purine (M Hocek et al, J Med Chem, 2000, 43,1817); mass spectrum (ESI) m/z 343[M+H]⁺

### Example 191

### 6-(9-Phenanthrenyl)-9-(β-D-ribofuranosyl)purine; mass spectrum (ESI) m/z 429 [M+H]⁺

### Example 192

### 9-(β-D-Ribofuranosyl)-6-(3-trifluoromethylphenyl)purine; mass spectrum (ESI) m/z 397[M+H]⁺

### Example 193

### 6-(2-Phenoxyphenyl)-9-(β-D-ribofuranosyl)purine; mass spectrum (ESI) m/z 421[M+H]⁺

### Example 194

### 6-(4-tert-Butylphenyl)-9-(β-D-ribofuranosyl)purine; mass spectrum (ESI) m/z 385[M+H]⁺

### Example 195

### 9-(β-D-Ribofuranosyl)-6-(2-trifluoromethoxyphenyl)purine; mass spectrum (ESI) m/z 413[M+H]⁺

### Example 196

### 6-(4-Phenoxyphenyl)-9-(β-D-ribofuranosyl)purine; mass spectrum (ESI) m/z 421 [M+H]⁺

### Example 197

### 6-(3-Methoxyphenyl)-9-(β-D-ribofuranosyl)purine (M Hocek et al, J Med Chem, 2000, 43,1817); mass spectrum (ESI) m/z 359[M+H]⁺

### Example 198

### 6-(2-Naphthyl)-9-(β-D-ribofuranosyl)purine; mass spectrum (ESI) m/z 379[M+H]⁺

### Example 199

### 6-(3-Biphenylyl)-9-(β-D-ribofuranosyl)purine; mass spectrum (ESI) m/z 405[M+H]⁺

### Example 200

### 6-[4-(2-Methylpropyl)phenyl]-9-(β-D-ribofuranosyl)purine; mass spectrum (ESI) m/z 385[M+H]⁺

### Example 201

### 6-(3-Fluorophenyl)-9-(β-D-ribofuranosyl)purine; mass spectrum (ESI) m/z 347[M+H]⁺

### Example 202

### 9-(β-D-Ribofuranosyl)-6-(4-trifluoromethylphenyl)purine; mass spectrum (ESI) m/z 397[M+H]⁺

### Example 203

### 9-(β-D-Ribofuranosyl)-6-(4-trifluoromethylphenyl)purine; mass spectrum (ESI) m/z 373[M+H]⁺

### Example 204

### 6-[3-(1-methyl)ethylphenyl]-9-(β-D-ribofuranosyl)purine; mass spectrum (ESI) m/z 371[M+H]⁺

### Example 205

### 9-(β-D-Ribofuranosyl)-6-(4-trifluoromethoxyphenyl)purine; mass spectrum (ESI) m/z 413[M+H]⁺

### Example 206

### 6-(4-Ethylphenyl)-9-(β-D-ribofuranosyl)purine; mass spectrum (ESI) m/z 357[M+H]⁺

### Example 207

Reaction of 2-amino-6-chloro-9-(2,3,5-tri-O-acetyl-β-D-ribofuranosyl)purine with phenylboronic acid followed by deprotection in an analogous manner to that described in example 181 gave 2-amino-6-phenyl-9-(β-D-ribofuranosyl)purine (M Hoceck, A. Holy, I. Votruba and H. Dvorakova, J. Med. Chem., 2000, 43, 1817) as a white solid of melting point 187-190° C.; mass spectrum (ESI) m/z 344[M+H]⁺.

### Example 208

A solution of 0.2 g of 2′3′5′-tri-O-benzoyl-5-ethyluridine in 1 ml of anhydrous methanol was treated with 0.05 ml of 1M sodium methoxide solution in methanol. The solution was stirred at room temperature for 2 hours. A few drops of glacial acetic acid was added and the mixture evaporated to dryness. The solid residue was purified by flash column chromatography on silica gel using ethyl acetate/isohexane for the elution to give 50 mg of 5-ethyluridine (C. Nakayama et al, J. Carbohyd. Nucleosides and Nucleotides, 1979, 6, 295) of melting point 180-181° C.; mass spectrum (ESI) 273[M+H]⁺.

The 2′3′5′-tri-O-benzoyl-5-ethyluridine used as the starting material was prepared as follows:

A mixture of 0.84 g of 5-ethyluracil, 2 mg of ammonium sulphate and 3.9 ml of hexamethyldisilazane was stirred under nitrogen and heated under reflux for 3.5 hours to give a clear solution. The solution was evaporated under reduced pressure to give an oil which was dissolved in 5 ml of anhydrous acetonitrile. This solution was added to a solution of 3.0 g of 1-O-acetyl-2,3,5-tri-O-benzoyl-D-ribose in 20 ml of anhydrous acetonitrile. The mixture was cooled in ice at <5° C. and treated with 1.4 ml of stannic chloride in three portions during 5 min then stirred at room temperature overnight. The mixture was treated with 12 ml of water and adjusted to pH 8 by addition of solid sodium bicarbonate. The resulting slurry was filtered through a pad of Hyflo and the filtered solid washed three times with dichloromethane. The combined filtrates were transferred to a separating funnel and the layers separated. The dichloromethane solution was dried over anhydrous sodium sulphate, filtered and evaporated to give 3.3 g of white solid residue. This was purified by flash column chromatography on silica gel using ethyl acetate/isohexane (1:1) for the elution to give 2.7 g of 2′3′5′-tri-O-benzoyl-5-ethyluridine as a white solid; mass spectrum (ESI) m/z 585[M+H]⁺.

In an analogous manner to that described in example 208 were prepared the following examples:

### Example 209

### 5-[(1-Methyl)ethyl]uridine (B. H. A. Knoblauch et al, Eur.J.Med.Chem.,1999, 34, 809)

### Example 210

### 5-Methoxymethyluridine (Patent No. JP57018696)

### Example 211

### 5-Ethoxymethyluridine

### Example 212

### 5-Chlorouridine (J. Asakura and M. J. Robins, J.Org.Chem., 1990, 55, 4928)

### Example 213

### 5-Methyl-1-(β-L-ribofuranosyl)uracil (A. Holy and F. Sorm, Collect. Czech. Chem. Commun., 1969, 34, 3383; mass spectrum (ESI) m/z 259[M+H]⁺

### Example 214

By the procedure of Nakayama et al, J. Carbohydr. Nucleosides, Nucleotides, 1979, 6, 295 was prepared 1-(β-D-arabinofuranosyl)-5-ethyluracil of melting point 164-165° C.

### Example 215

A solution of 3.0 g of 1-(β-D-arabinofuranosyl)uracil and 3.0 g of N-bromosuccinimide in 20 ml of N,N-dimethylformamide was stirred at room temperature for 1 hour. The solution was evaporated to dryness and the residual yellow oil stirred with a mixture of ethanol and chloroform (4:1) until a fine solid crystallised. After cooling the solid was filtered off washed with ethanol and diethyl ether and dried to give 2.3 g of 1-(β-D-arabinofuranosyl)-5-bromouracil, (R. F. Shinazi et al, J. Med. Chem.,1979, 22, 1273). Recrystallisation from ethanol gave analytically pure material with melting point 227° C. (decomposition).

### Example 216

By the procedure of K. Felczak, et al, Nucleosides and Nucleotides, 1993, 12, 245 was prepared 5-methyl-4-thiouridine.

### Example 217

By the procedure of A. Miah et al., Nucleosides and Nucleotides, 1997, 16, 53 was prepared 4-methoxy-1-(β-D -ribofuranosyl)pyrimidin-2(1H)-one.

### Example 218

By the procedure of K. H. Scheit, Tet. Lett., 1967, 113 was prepared 4-(methylthio)-1-(□-D-ribofuranosyl)pyrimidin-2(1H)-one; mass spectrum (ESI) m/z 275 [M+H]⁺.

### Example 219

By an analogous procedure to that of K. H. Scheit, Tet. Lett., 1967, 113 was prepared 5-fluoro-4-methylthio-1-1-(□-D-ribofuranosyl)pyrimidin-2(1H)-one; mass spectrum (ESI) m/z 293 [M+H]⁺.

### Example 220

By an analogous procedure to that of K. H. Scheit, Tet. Lett., 1967, 113 was prepared 5-methyl-4-methylthio-1-(□-D-ribofuranosyl)pyrimidin-2 (1H)-one; mass spectrum m/z 289 [M+H]⁺.

### Example 221

By a procedure analogous to that of Fox et al., Tet. Lett. 1966, 4927 was prepared 5-fluoro-4-thiouridine.

### Example 222

By the procedure of Hoffer et al., J.Amer.Chem.Soc., 1959,81,4112 was prepared 1-(2-deoxy α D erthyro pentofuranosyl)-5-fluorouracil.

### Example 223

By the procedure of Zemlicka et al., J.Amer.Chem.Soc.,1972, 94,3213 was prepared 2′-Deoxy-5-fluoro-3-methyluridine.

### Example 224

By an analogous procedure to that of Zemlicka et al., J.Amer.Chem.Soc.,1972, 94, 3213 was prepared 1-(α-D-erthyro-2-deoxypentofuranosyl)-5-fluoro-3-methyluracil, (D. J. Adams and G. W. Gooday, Mach. Naturwiss.Tech., 1983, 39).

### Example 225

A stirred slurry of 1.0 g of O2,2′-anhydrouridine in 22 ml of anhydrous chloroform was saturated with hydrogen chloride gas for 5 hours. The solid was filtered off dried and suspended in 150 ml of 1,4-dioxane. The suspension was heated at 75° C. under nitrogen until a solution was obtained. After cooling this was evaporated and the residual syrup triturated with 50 ml of boiling ethyl acetate. A solid formed which was broken up. After cooling the product was filtered to give 1.05 g of 2′-chloro-2′-deoxyuridine (Tetrahedron 1977, 33, 2131). Recrystallisation from ethanol gave analytically pure material of melting point 206-207° C.

The O-2,2′-anhydrouridine used as the starting material was prepared as follows:

A mixture of 10.0 g of uridine, 11.4 g of diphenyl carbonate, 0.2 g of sodium hydrogen carbonate and 20 ml of N,N-dimethylformamide was stirred under nitrogen and heated at 155° C. for 30 min. The solution was cooled and added dropwise to 200 ml of anhydrous diethyl ether. After stirring the mixture overnight the precipitated solid was filtered off and washed with methanol and dried to give 6.3 g of O2,2′-anhydrouridine of melting point 241-244° C.

### Example 226

A saturated solution of hydrogen bromide in 30 ml of trifluoroacetic acid was treated with 1.0 g of O2,2′-anhydrouridine. The mixture was stirred for 4 days at room temperature in a sealed flask. The resulting solution was evaporated to dryness to yield a brown syrup which crystallised on standing. Recrystallisation from ethanol gave 2′-bromo-2′-deoxyuridine (Codington et al, J. Org. Chem., 1964, 29, 558) of melting point 194-195° C.

### Example 227

By the procedure of J. J. Fox and N. C. Miller, J. Org. Chem., 1963, 28, 936 was prepared 1-(2-deoxy-β-D-lyxofuranosyl)-5-methyluracil of melting point 170-171° C.

### Example 228

By the procedure of Johansson et al., U.S. Pat. No. 5,506,215 was prepared 3′-deoxy-3′-fluoro-5-methyluridine.

### Example 229

A suspension of 2.0 g of 2′-deoxy-5-ethyl-5′-O-triphenylmethyluridine in 20 ml of benzene and 6.5 ml of 1,4-dioxane was stirred and treated with 0.5 ml of iodomethane and 0.45 g of powdered potassium hydroxide. The mixture was stirred and heated at 40° C. for 5 hours then evaporated and the residue dissolved in 2 ml of methanol and poured into 100 ml of water. The resulting white emulsion was extracted with four 100 ml portions of chloroform. The extracts were dried, filtered and evaporated and the residue redissolved in 20 ml of 80% acetic acid. The solution was heated at 100° C. for 1 hour then evaporated to dryness. The residue was purified by flash column chromatography on silica gel using ethyl acetate for the elution to give 0.25 g of 2′,3′-dideoxy-5-ethyl-3′-methoxyuridine. Recrystallisation from a mixture of ethyl acetate and hexane gave analytically pure material of melting point 118-127° C.

The 2′-deoxy-5-ethyl-5′-O-triphenylmethyluridine used as the starting material was prepared as follows:

A solution of 15.7 g of 2′-deoxy-5-ethyluridine and 20.4 g of chlorotriphenylmethane in 290 ml of dry pyridine was stirred under nitrogen and heated at 100° C. for 30 min. The mixture was cooled and poured into 31 of ice/water and extracted with three 500 ml portions of ethyl acetate, the combined extracts were washed with 1.5 l of water then dried and evaporated. The residue was taken up in 30 ml of acetone and 210 ml of hot toluene added. The acetone was removed by boiling on a hot water bath. After cooling at −20° C. the precipitate was filtered off and washed with diethyl ether to give 19.5 g of 2′-deoxy-5-ethyl-5′-O-triphenylmethyluridine of melting point 168-172° C.

### Example 230

By the procedure of Griffin and Todd, J. Chem. Soc., 1958, 1391 was prepared 5′-benzyloxy-2′,3′-dideoxy-5-methyluridine of melting point 140° C. (decomposition).

### Example 231

By the procedure of C. K. Chu et al, J. Med. Chem., 1989, 32, 612 was prepared 2′,3′-dideoxy-5-ethyl-3′-iodouridine of melting point 161.5-163.5° C.

### Example 232

By the procedure of C. K. Chu et al, J. Med. Chem., 1989, 32, 612 was prepared 3′-azido-2′,3′-dideoxy-5-ethyluridine of melting point 116-118° C.

### Example 233

A solution of 2.0 g of 1-(5-O-acetyl-3-azido-2,3-dideoxy-1-β-D-ribofuranosyl)-5-methyl-4-(1,2,4-triazol-1-yl)pyrimidin-2(1H)-one in 23 ml of dioxane was treated with 3.5 ml of concentrated (32%) aqueous ammonia solution and the mixture stirred at room temperature for 6 hours. The solution was evaporated and the residue dissolved in 36 ml of a saturated solution of ammonia in methanol which was stirred at room temperature for 3 days. The residue was extracted several times with boiling ethyl acetate. The combined ethyl acetate extracts were filtered and evaporated. The residue was dissolved in ethanol and the solution concentrated to low volume then diluted with ether. The gum which separated crystallised and the solid was filtered to give 0.47 g of 3′-azido-2′,3′-dideoxy-5-methylcytidine (T. S. Lin et al, J.Med.Chem.,1983, 26, 1691) of melting point 85-88° C.

The 1-(5-O-acetyl-3-azido-2,3-dideoxy-1-β-D-ribofuranosyl)-5-methyl-4-(1,2,4-triazol-1-yl)pyrimidin-2(1H)-one used as the starting material was prepared as follows:

A solution of 1.34 g of 3′-azido3′-deoxythymidine in 13.5 ml of anhydrous pyridine was treated with 0.76 ml of acetic anhydride and the mixture stirred at room temperature overnight. 2.5 ml of methanol was added and the solution stirred for 30 min then evaporated to dryness. The residue was taken up in 125 ml of dichloromethane and the solution washed with 50 ml of 1 m hydrochloric acid, 25 ml of saturated sodium hydrogen carbonate solution and 25 ml of water then dried over anhydrous sodium sulphate, filtered and evaporated to give 1.46 g of 5′-O-acetyl-3′-azido-3′-deoxythymidine as a colorless gum which was used without further purification.

A suspension of 1.68 g of 1,2,4-triazole in 28 ml of anhydrous acetonitrile was stirred and heated to 50° C. to give a clear solution. This was removed from the heating bath and stirred while 0.97 ml of phosphorus oxychloride was added dropwise during 5 min so that the temperature of the reaction mixture was maintained at 50-52° C. A crystalline white precipitate separated. The mixture was stirred at room temperature for 15 min then cooled to 5° C. in ice while 6.42 ml of anhydrous triethylamine was added dropwise at 5-10° C. during 3 min. The mixture was stirred for a further 15 min at room temperature then a solution of 1.68 g of crude 5′-O-acetyl-3′-azido-3′-deoxythymidine in 17 ml of anhydrous acetonitrile was added over 3 min. The mixture was stirred at room temperature overnight then treated with 4.34 ml of triethylamine and 1.08 ml of water. The mixture was stirred for 10 min then evaporated to dryness and the residue taken up in 125 ml of dichloromethane. The solution was washed with saturated sodium hydrogen carbonate solution then evaporated to a yield 2.0 g of 1-(5-O-acetyl-3-azido-2,3-dideoxy-1-β-D-ribofuranosyl)-5-methyl-4-(1-(1,2,4-triazol-1-yl)pyrimidin-2(1H)-one as a crystalline solid which was used without further purification.

### Example 234

By the procedure of G. Gosselin, et al, Patent No. WO 0025799 was prepared 1-(3-deoxy-β-L-threo-pentofuranosyl)-5-fluorocytosine.

### Example 235

By the procedure of R. Saladino et al, Tetrahedron, 1996, 52, 6759 was prepared 4-methylamino-1-(β-D-ribofuranosyl)pyrimidin-2(1H)-one; mass spectrum (ESI) m/z 258 [M+H]⁺.

### Example 236

By the procedure of T. Kulikowski and D. Shugar, Acta. Biochim. Pol., 1979, 26, 145 was prepared 5-fluoro-4-methylamino-1-(β-D-ribofuranosyl)pyrimidin-2(1H)-one; mass spectrum (ESI) m/z 276[M+H]⁺.

### Example 237

A solution containing 1.5 g of cytidine and 0.86 g of 2,5-dimethoxytetrahydrofuran in 10 ml glacial acetic acid was heated under nitrogen at 110° C. for 1 hour. The solvents were evaporated under low vacuum to give a lilac solid, which was purified by flash chromatography on silica-gel using methanol/dichloromethane (1:19) for the elution to give 90 mg of 4-(1-pyrrolyl)-1-(β-D-ribofuranosyl)pyrimidin-2(1H)-one as a white solid; mass spectrum (ESI) m/z 294 [M+H]⁺.

### Example 238

A solution of 0.3 g of 1-(2,3,5-tri-O-benzoyl-β-L-ribofuranosyl)-4(3H)-oximinopyrimidin-2(1H)-one in 5 ml of anhydrous methanol was treated with 0.2 ml of a 1M solution of sodium methoxide in methanol and stirred at room temperature for 24 hours. The mixture was evaporated to dryness and the residue purified by flash chromatography on silica gel using methanol/dichloromethane 1:9 for the elution to give 79 mg of 4(3H)-oximino-1-(-L-ribofuranosyl)pyrimidin-2(1H)-one as a white solid of melting point 138-139° C.; mass spectrum m/z 260[M+H]⁺.

The 1-(2,3,5-tri-O-benzoyl-β-L-ribofuranosyl)-4(3H)-oximinopyrimidin-2 (1H)-one used as the starting material was prepared as follows:

A mixture of 1.0 g of uracil and 1.5 g of 1-O-acetyl-2,3,5-tri-O-benzoyl-L-ribose in 50 ml of anhydrous acetonitrile was treated with 2.21 ml of N,O-bis(trimethylsilyl)acetamide and heated at 76° C. under nitrogen until a solution was obtained. To the solution was added 0.98 g of trimethylsilyl trifluoromethane sulphonate and heating at 70° C. then continued overnight. The mixture was cooled, diluted with 500 ml of dichloromethane and washed three times with 50 ml of saturated sodium hydrogen carbonate solution. The dichloromethane solution was washed with brine, dried over anhydrous sodium sulphate, filtered and evaporated to give 1.61 g of 2′,3′,5′-tri-O-benzoyl-L-uridine as a white solid; mass spectrum m/z 557 [M+]⁺.

A solution of 1.80 g of 1,2,4-triazole was prepared in 25 ml of anhydrous acetonitrile by warming. The solution was stirred at room temperature under nitrogen while 0.86 g of phosphorus oxychloride was added. A white suspension was obtained which was cooled to 5° C. in ice and treated with 3.46 ml of triethylamine during 4 min followed dropwise by a solution of 1.61 g of 2′,3′,5′-tri-O-benzoyl-L-uridine in 25 ml of anhydrous acetonitrile during 2 min. The mixture was stirred at room temperature for 2.5 hours then treated with a further 2.41 ml of triethylamine followed by 0.63 ml of water and stirred for 10 min. The mixture was diluted with 150 ml of dichloromethane and washed with a 10% solution of sodium hydrogen carbonate and brine. The dichloromethane solution was dried over anhydrous sodium sulphate, filtered and evaporated to give 1.6 g of a yellow powder. This was purified by flash chromatography on silica gel using ethyl acetate/isohexane (1:9) for the elution to give 1.12 of 1-(2,3,5-tri-O-benzoyl-β-L-ribofuranosyl)-4-(1,2,4-triazol-1-yl)pyrimidin-2(1H)-one as a white solid of melting point 83-84° C.; mass spectrum m/z 608 [M+H]⁺.

A suspension of 0.43 g of hydroxylamine hydrochloride in 15 ml of anhydrous methanol was treated with 4.96 ml of a 1M solution of sodium methoxide in methanol. After stirring for 10 min a solution of 0.75 g of 1-(2,3,5-tri-O-benzoyl-β-L-ribofuranosyl)-4-(1,2,4-triazol-1-yl)pyrimidin-2 (1H)-one in a mixture of 20 ml of methanol and 20 ml of tetrahydrofuran was added and the mixture stirred at room temperature overnight. The mixture was evaporated and the residue purified by flash chromatography on silica gel using methanol/dichloromethane 1:24 for the elution to give 0.605 g of 1-(2,3,5-tri-O-benzoyl-β-L-ribofuranosyl)-4(3H)-oximinopyrimidin-2(1H)-one as a white solid; mass spectrum m/z 572[M+H]⁺.

### Example 239

In an analogous manner to that described in example 238 was prepared 4-oximino-1-(β-D-ribofuranosyl)pyrimidin-2(1H)-one (I.Wempen et al, J.Med.Chem.,1968, 11, 144); mass spectrum (ESI) m/z 260[M+H]⁺.

### Example 240

In an analogous manner to that described in example 238 was prepared 4-oximino-1-(β-D-arabinofuranosyl)pyrimidin-2(1H)-one, (I. Wempen et al, J.Med.Chem.,1968, 11, 144).

### Example 241

In an analogous manner to that described in example 238 was prepared 5-fluoro-4-oximino-1-(β-D-ribofuranosyl)pyrimidin-2(1H)-one); mass spectrum m/z 319 [M+H]⁺.

### Example 242

By the procedure of S. L. Anliker et al, J. Pharm. Sci., 1994, 83, 716 was prepared 1-(2-deoxy-2,2-difluoro-α-D-erythropentofuranosyl)uracil.

### Example 243

By the procedure of S. L. Anliker et al, J. Pharm. Sci., 1994, 83, 716 was prepared 1-(2-deoxy-2,2-difluoro-β-D-erythropentofuranosyl)cytosine.

### Example 244

By the procedure of E Moyroud and P Strazewcki, Tetrahedron, 1999, 55, 1277 was prepared L-cytidine or according the following experimental method:

A solution of 0.40 g of 1-(2,3,5-tri-O-benzoyl-β-L-ribofuranosyl)-4-(1H-1,2,4-triazol-1-yl)-1H-pyrimidin-2-one in 10 ml of 1,4-dioxane was treated with 0.5 ml of 35% aqueous ammonia solution and stirred at room temperature for 12 hours. The reaction mixture was concentrated under reduced pressure to leave a white solid which was purified by flash chromatography on silica gel using dichloromethane/methanol (1:24 then 1:9) to give 0.22 g of 2′,3′,5′-tri-O-benzoyl-L-cytidine. This was dissolved in 2 ml of anhydrous methanol and treated with 100 μl of 1M sodium methoxide solution. The reaction mixture was stirred for 16 hours then evaporated and the residue purified by flash chromatography on silica gel using dichloromethane/methanol(9:1 then 3:2) for the elution to give 80 mg of L-cytidine as a white solid; mass spectrum(ESI) m/z 301 [M+H+MeCN]⁺.

The 1-(2,3,5-tri-O-benzoyl-p -L-ribofuranosyl)-4-(1H-1,2,4-triazol-1-yl)-1H-pyrimidin-2-one used as the starting material was prepared as follows:

A mixture containing 1.0 g of uracil and 1.5 g of 1-O-acetyl-2,3,5-tri-O-benzoyl-L-ribose in 50 ml of anhydrous acetonitrile was treated with 1.82 g of N,O-bis-trimethylsilylacetamide and heated at 76° C. under nitrogen until a clear solution was obtained. 0.98 g of trimethylsilyl trifluoromethanesulphonate was then added in one portion and heating at 70° C. continued for 16 hours. The mixture was cooled and diluted with 500 ml of dichloromethane. The solution was washed three times with 50 ml of saturated sodium hydrogen carbonate solution and brine then dried over anhydrous sodium sulphate, filtered and evaporated to give 1.61 g of 2′,3′, 5′-tri-O-benzoyl-L-uridine as a white solid which was used without further purification.

To a solution of 1.80 g of 1,2,4-triazole in 25 ml of anhydrous acetonitrile at room temperature under nitrogen was added 0.86 g of phosphorus oxychloride. The mixture was cooled in a bath of ice and stirred for 15 min then treated with 2.53 g (3.46 ml) of triethylamine during 4 min. The ice bath was removed and a solution of 1.61 g of 2′,3′,5′-tri-O-benzoyl-L-uridine in 25 ml of anhydrous acetonitrile added dropwise during 2 min. The reaction mixture was stirred at room temperature under nitrogen for 2.5 hours then a further 2.41 ml of triethylamine added followed by 0.63 ml of water. After stirring for 10 min the reaction mixture was diluted with 150 ml of dichloromethane and washed with a 10% aqueous solution of sodium hydrogen carbonate. The dichloromethane solution was washed with brine then dried over anhydrous sodium sulphate. Evaporation gave 1.61 g of solid which was purified by flash chromatography on silica gel using ethyl acetate/isohexane (1:9) for the elution to give 1.12 g of 1-(2,3,5-tri-O-benzoyl-β-L-ribofuranosyl)-4-(1H-1,2,4-triazol-1-yl)-1H-pyrimidin-2-one as a white solid; mass spectrum (ESI) m/z 608 [M+H]⁺.

### Example 245

A solution of 55 mg of 1-[1(R)-2,2-difluoro-3(R)-acetoxy-4(R)-(acetoxymethyl)-cyclopentyl]-4-(1H-1,2,4-triazol-1-yl)-1H-pyrimidin-2-one in 35% aqueous ammonia was stirred at room temperature for 14 hours. The reaction mixture was concentrated under reduced pressure and the residue purified by chromatography on silica gel using dichloromethane/methanol (5:1) for the elution to give 35 mg of 1-[1(R)-2,2-difluoro-3(R)-hydroxy-4(R)-(hydroxymethyl)cyclopentyl]-4-amino-1H-pyrimindin-2-one as colorless crystals; mass spectrum (ESI) m/z 262 [M+H]⁺; ¹H NMR (270 MHz, DMSO-d₆) 1.71(1H, m), 1.90 (1H, m), 1.99 (1H, m), 3.45(1H, m), 3.55 (1H, m), 3.80 (1H, m), 4.73 (1H, t), 5.22 (1H, m), 5.68 (1H, d), 5.71 (1H, d), 7.15 (1H, br.s), 7.18 (1H, br.s), 7.56 (1H, d).

The 1-[1(R)-2,2-difluoro-3(R)-acetoxy-4(R)-(acetoxymethyl)-cyclopentyl]-4-(1H-1,2,4-triazol-1-yl)-1H-pyrimidin-2-one used as the starting material was prepared as follows:

A solution of 28 g of (3aS,4R,7S,7aR)-tetrahydro-2,2-dimethyl-4,7-methano-1,3dioxolo[4,5-c]pyridin-6(3aH)-one, in 300 ml of 10% methanolic hydrogen chloride was stirred at ambient temperature for 3 days. The reaction mixture was concentrated under reduced pressure to ca 100 ml and cooled in a refrigerator. The white precipitate was collected and washed with methanol to give a first crop of 25.44 g of (1S,2R,3S,4R)-4-amino-2,3-dihydroxy-cyclopentanecarboxylic acid methyl ester hydrochloride. The combined mother liquor and washings were concentrated and recrystallised from methanol to give 4.30 g of a second crop; ¹H NMR (270 MHz, DMSO-d₆) 1.68 (1H, dddd), 2.22 (1H, dddd), 3.2-3.35 (1H, br.m), 3.62 (3H, s), 3.80-3.90 (1H, br.m), 4.00-4.10 (1H, br.m), 5.20 (1H, br.s), 5.30 (1H, br.s), 8.39 (3H, br.s).

To a solution of 28.6 g of (1S,2R,3S,4R)-4-amino-2,3-dihydroxy-cyclopentanecarboxylic acid methyl ester hydrochloride and 35.36 g of di-t-butyl dicarbonate in 400 ml of dioxane was added 27.2 g of sodium hydrogen carbonate dissolved in a minimum volume of water and the reaction mixture was stirred at ambient temperature for 36 hours. The reaction mixture was filtered and the filter washed thoroughly with 300 ml of acetone. The filtrate and washings were concentrated under reduced pressure to ca 100 ml and the residue partitioned between 300 ml of ethyl acetate and 100 ml of water. The water layer was extracted further with 300 ml of ethyl acetate. The combined organic extracts were dried over anhydrous sodium sulphate, filtered and concentrated under reduced pressure. The residue was recrystallised from 200 ml of diethyl ether to give 34.9 g of (1S,2R,3S,4R)-4-t-butoxycarbonylamino-2,3-dihydroxy-cyclopentanecarboxylic acid methyl ester as colorless crystals; H¹ NMR (270 MHz, CDCl₃) 1.45 (9H, s), 1.60-1.75 (1H, m), 2.35-2.45 (1H, m), 2.93 (1H, ddd), 3.10 (1H, br.s), 3.71 (3H, s), 3.80-3.95 (2H, m), 4.28 (1H, m), 4.65 (1H, br.s), 4.88 (1H, br.s).

To a solution of 33.77 g of (1S,2R,3S,4R)-4-t-butoxycarbonylamino-2,3-dihydroxy-cyclopentanecarboxylic acid methyl ester in 300 ml of anhydrous tetrahydrofuran was added dropwise a solution of 4.0 g of lithium borohydride in 100 ml of anhydrous tetrahydrofuran and the reaction mixture stirred for 2 hours at ambient temperature. The excess lithium borohydride was decomposed by addition of 10 ml of water and stirring for a short time. The reaction mixture was dried over anhydrous sodium sulphate, filtered and the filter washed thoroughly with tetrahydrofuran. The combined filtrate and washings were concentrated under reduced pressure and dried under vacuum to give crude (1R,2S,3R,5R)-3-t-butoxycarbonylamino-5-hydroxymethyl-cyclopentan-1,2-diol which was redissolved in 100 ml of dioxane and treated dropwise with 300 ml of a 4M solution of hydrogen chloride in dioxane. The reaction mixture was stirred at ambient temperature for 14 hours. The solvent and volatile materials were removed by purging with nitrogen gas and then evaporation under reduced pressure. The residue was rinsed twice with 100 ml of n-hexane then dried under vacuum to give crude (1R,2S,3R,5R)-3-amino-5-hydroxymethyl-cyclopentan-1,2-diol hydrochloride. A solution of this and 22.8 g of 2,4-dinitro-fluorobenzene in 100 ml of absolute N,N-dimethyl formamide was treated with sodium hydrogen carbonate and the suspension stirred at ambient temperature for 5 hours. The reaction mixture was filtered and the filter washed thoroughly with methanol. The combined filtrate and washings were concentrated under reduced pressure and the residue purified by chromatography on silica gel using dichloromethane/methanol(9:1 to 4:1) for the elution to give 30.15 g of (1R,2S,3R,5R)-3-[(2,4-dinitrophenyl)amino)-5-hydroxymethyl-cyclopentan-1,2-diol as an amorphous yellow solid; ¹H NMR (270 MHz, DMSO-d₆) 1.32 (1H, ddd), 1.95-2.05 (1H, m), 2.35 (1H, ddd), 3.44 (2H, s), 3.70-3.85 (2H, m), 3.99 (1H, ddd), 4.62 (1H, br.t), 4.78 (1H, br.d), 5.03 (1H, br.d), 7.33 (1H, d), 8.27 (1H, dd), 8.67 (1H, d), 8.86 (1H, d).

To a solution of 30.15 g of (1R,2S,3R,5R)-3-[(2,4-dinitrophenyl)amino)-5-hydroxymethyl-cyclopentan-1,2-diol and 19.69 g of imidazole in 150 ml of dry N,N-dimethylformamide was added in portions tetra-isopropyl dichlorosiloxane. The reaction mixture was stirred at room temperature under argon for 14 hours then poured into 500 ml of water and extracted twice with 400 ml of ethyl acetate. The combined organic extracts were washed twice with 300 ml of brine, dried over anhydrous sodium sulphate, filtered and evaporated to give yellow sticky crystals which were recrystallised from n-hexane to give 43.23 g of 2[(2,4-dinitrophenyl)amino-5,5,7,7-tetraisopropyl-hexahydro-4,6,8-trioxa-5,7-disilacyclopentacyclooctene-3-ol in two crops.

To a solution of 2.0 g of 2[(2,4-dinitrophenyl)amino-5,5,7,7-tetraisopropyl-hexahydro-4,6,8-trioxa-5,7-disila-cyclopentacyclooctene-3-ol in 15 ml of dry acetonitrile was added 4.0 g of 1,1,1-triacetoxy-1,1-dihydro-1,2-benziodoxol-3(1H)-one and the suspension stirred at 40° C. under argon for 14 hours. The reaction mixture was diluted with 40 ml of saturated sodium hydrogen carbonate solution and extracted twice with 50 ml of dichloromethane. The combined organic extracts were washed successively with 40 ml of saturated sodium hydrogen carbonate solution and 40 ml of brine then dried over anhydrous sodium sulphate, filtered and evaporated. The yellow amorphous residue which was purified by chromatography on silica gel using n-hexane/ethyl acetate (4:1) for the elution to give 1.50 g of 2-[(2,4-dinitrophenyl)amino)-5,5,7,7-tetraisopropyl-hexahydro-4,6,8-trioxa-5,7-disila-cyclopentacyclooctene-3-one; ¹H NMR (270 MHz, CDCl₃)1.02-1.15 (28H, m), 1.55-1.62(1H, br.m), 2.16-2.28(1H, m), 2.54-2.66 (1H, m), 3.93 (1H, dd), 4.14 (1H, dd), 4.20 (1H, m), 4.30 (1H, d), 7.17 (1H, d), 8.28 (1H, dd), 8.63 (1H, br.d), 9.14 (1H, d).

To an ice-cooled solution of 6.24 ml of diethylamino sulphur trifluoride complex in 24 ml of dry dichloromethane was added dropwise over 10 min a solution of 2.0 g of 2-[(2,4-dinitrophenyl)amino)-5,5,7,7-tetraisopropyl-hexahydro-4,6,8-trioxa-5,7-disila-cyclopentacyclooctene-3-one in 24 ml of dry dichloromethane. The mixture was stirred at 0° C. under argon for 4 hours then poured into 100 ml of sodium hydrogen carbonate solution and extracted three times with 100 ml of dichloromethane. The combined extracts were washed successively with three portions of 200 ml of sodium bicarbonate solution and twice with 100 ml of brine then dried over anhydrous sodium sulphate, filtered and evaporated . The dark yellow amorphous residue was purified by chromatography on silica gel using n-hexane/dichloromethane (1:1) for the elution to give 0.59 g of (3,3-difluoro-5,5,7,7-tetraisopropyl-hexahydro-4,6,8-trioxa-5,7-disilacyclopentacyclooctene-2-yl) (2,4-dinitrophenyl)amine; ¹H NMR (270 MHz, CDCl3) 1.02-1.15 (28H, m), 1.60-1.72 (1H, br.m), 2.02-2.16 (1H, m), 2.36-2.48 (1H, m),3.80 (1H, dt), 4.05 (1H, dd), 4.10-4.20 (2H, m), 7.05 (1H, d), 8.28 (1H, dd), 8.50 (1H, br.d), 9.14 (1H, d).

To an ice-cooled solution of 0.677 g of (3,3-difluoro-5,5,7,7-tetraisopropyl-hexhydro-4,6,8-trioxa-5,7-desilacyclopentacyclooctene-2-yl)-(2,4-dinitrophenyl)amine in 15 ml of tetrahydrofuran was added 2.5 ml of a 1M solution of tetrabutylammonium fluoride in tetrahydrofuran and the reaction mixture was stirred at 0° C. under an atmosphere of argon for 3 hours. The reaction mixture was concentrated under reduced pressure and the residue was partitioned between 40 ml of ethyl acetate and 50 ml of water. The water layer was extracted further with three portions of 40 ml of ethyl acetate. The combined extracts were washed with 30 ml of brine, dried over anhydrous sodium sulphate, filtered and evaporated. The residue was purified by chromatography on silica gel using dichloromethane/methanol (20:1) for the elution to give 0.364 g of (1R, 3R, 5R)-3-[(2,4-dinitrophenyl)amino]-2,2-difluoro-5-(hydroxymethyl)cyclopentanol as a pale yellow solid; ¹H NMR (270 MHz, CDCl₃) 1.70 (1H, m), 2.22 (1H, m), 2.54 (1H, m), 3.70-3.90 (3H, m), 4.18 (1H, m), 4.40 (1H, m), 4.66 (1H, d), 7.21 (1H, d), 8.31 (1H, dd), 8.78 (1H, br.d), 9.11 (1H, d).

To a solution of 0.36 g of (1R, 3R, 5R)-3-[(2,4-dinitrophenyl)amino]-2,2-difluoro-5-(hydroxymethyl)cyclopentanol in 20 ml of 75% aqueous acetone was treated with 1.0 g of Dowex-1 ion-exchange resin, which had been thoroughly washed successively with 1M sodium hydroxide solution, distilled water and methanol prior to use. The reaction mixture was stirred at ambient temperature for 24 hours. The resin was filtered off and thoroughly washed with approximately 100 ml of 75% aqueous acetone. The combined filtrate was concentrated under reduced pressure to remove acetone and the resulting aqueous solution acidified with 2 ml 1M hydrochloric acid. The aqueous solution was washed twice with 20 ml of ethyl acetate then lyophilised to give 0.134 g of (1R, 3R, 5R)-3-amino-2,2-difluoro-5-(hydroxymethyl)-cyclopentanol hydrochloride as a colorless powder.

To a solution of 0.127 g of (1R, 3R, 5R)-3-amino-2,2-difluoro-5-(hydroxymethyl)-cyclopentanol hydrochloride in 2 ml of anhydrous N,N-dimethylformamide were added freshly desiccated 4° A molecular sieves. The mixture was stirred at −30° C. for 30 minutes then treated with 2.5 ml of a 0.427M solution of 3-ethoxy-2-propenoyl isocyanate. The mixture was stirred at at −30° C. for 30 minutes and then at room temperature fro 14 hours. The reaction mixture was concentrated under reduced pressure and the residue purified by chromatography on silica gel using dichloromethane/methanol (9:1 then 5:1)) for the elution to give 0.157 g of 1-[1(R)-2,2-difluoro-3(R)-hydroxy-4(R)-(hydroxymethyl) cyclopentyl]-3 (3-ethoxy-E-2-propenoyl)urea as a colorless solid; ¹H NMR (270 MHz, DMSO-d₆) 1.23 (3H, t), 1.78-1.91 (1H, m), 2.04-2.15 (1H, m), 3.38-3.45 (2H, m), 3.60-3.75 (1H, m), 3.96 (2H, q), 4.22-4.44 (1H, m), 4.72 (1H, t), 5.51 (1H, d), 5.63 (1H, d), 7.59 (1H, d), 8.80 (1H, s). 10.21 (1H, s).

A solution of 0.15 g of 1-[1(R)-2,2-difluoro-3(R)-hydroxy-4(R)-(hydroxymethyl)cyclopentyl]-3(3-ethoxy-E-2-propenoyl)urea in 4 ml of 5% aqueous sulphuric acid was boiled under reflux for 3 hours. The reaction mixture was neutralised by addition of sodium hydroxide solution then concentrated under reduced pressure. The residue was suspended in 35 ml of absolute ethanol and filtered. The material on the filter was washed three times with 35 ml of absolute ethanol and the combined filtrate concentrated under reduced pressure to give 0.215 g of crude 1-[1(R)-2,2-difluoro-3(R)-hydroxy-4(R)-(hydroxymethyl)cyclopentyl]-1H-pyrimidine-2,4-dione as a colorless powder which was used without further purification.

To a solution of 0.215 g of crude 1-[1(R)-2,2-difluoro-3(R)-hydroxy-4(R)-(hydroxymethyl)cyclopentyl]-1H-pyrimidine-2,4-dione in 3 ml of acetic anhydride was added 5 mg of 4-dimethylaminopyridine and the reaction mixture stirred at room temperature for 14 hours. The mixture was concentrated under reduced pressure and the residue partitioned between 30 ml of ethyl acetate and sodium hydrogen carbonate solution. The aqueous layer was extracted twice more with 30 ml of ethyl acetate. Combined extracts were washed with 30 ml of brine, dried over anhydrous sodium sulphate, filtered and evaporated. The residue was triturated with t-butyl methyl ether to give 0.148 g of 1-[1(R)-2,2-difluoro-3(R)-acetoxy-4(R)-(acetoxymethyl)cyclopentyl]-1H-pyrimidine-2,4-dione, which was used without further purification.

To a solution of 0.128 g of 1-[1(R)-2,2-difluoro-3(R)-acetoxy-4(R)-(acetoxymethyl)cyclopentyl]-1H-pyrimidine-2,4-dione and 0. 128 g of 1,2,4-1H-triazole in dry pyridine was added dropwise 180 μl of 4-chlorophenyl dichlorophosphate and the reaction mixture stirred at room temperature for 14 hours. The reaction mixture was concentrated under reduced pressure and the residue partitioned between 30 ml of ethyl acetate and sodium hydrogen carbonate solution. The aqueous layer was extracted twice more with 30 ml of ethyl acetate and the combined extracts washed with 30 ml of brine, then dried over anhydrous sodium sulphate, filtered and evaporated. The residue was purified by chromatography using ethyl acetate for the elution to give 0.112 g of 1-[1(R)-2,2-difluoro-3(R)-acetoxy-4(R)-(acetoxymethyl)cyclopentyl]-4-(1H-1,2,4-triazol-1yl)-1H-pyrimidin-2-one; ¹H NMR (270 MHz, CDCl₃) 1.86 (1H, m), 2.11 (3H, s), 2.18 (3H, s), 2.30-2.50 (1H, m), 2.50-2.70 (1H, m), 4.15-4.25 (2H, m), 5.19 (1H, ddd), 5.55-5.75 (1H, m), 7.12(1H, d), 7.93 (1H, d), 8.15 (1H, s), 9.29 (1H, s).

### Example 246

Starting with 1(R)-amino-2(S),3(R)-diacetoxy-4(R)-acetoxymethylcyclopentane in manner analogous to that described by Y. F. Shealy and C. A. O'Dell, J. Heterocyclic Chem., 1980,17, 353 was prepared 4-amino-1(R)-(2(S),3(R)-dihydroxy-4(R)-hydroxymethyl-cyclopentyl)-1H-pyrimidin-2-one; mass spectrum(ESI) m/z 242 [M+H]⁺.

### Example 247

The compound may be prepared according to G. Gosselin et al, J. Med. Chem. 1987, 30960, 982. A solution of 0.283 g of 1-(3-O-acetyl-2,5-bis-O-t-butyldimethylsilyl-β-D-xylofuranosyl)cytosine and 0.245 g of ammonium fluoride in 5 ml of anhydrous methanol was stirred and heated at 50-60° C. under nitrogen for 24 hours. The solution was evaporated and the white solid residue purified by flash chromatography on silica gel using methanol/dichloromethane (1:19 to 2:3) for the elution to give 50 mg of 1-(β-D-xylofuranosyl)cytosine; mass spectrum(ESI) m/z 244 [M+H]⁺.

The 3-O-acetyl-2,5-bis-O-t-butyldimethylsilyl-1-(β-D-xylofuranosyl)cytosine used as the starting material was prepared as follows:

A solution of 0.5 g of 1-(2,5-bis-O-t-butyldimethylsilyl-β-D-xylofuranosyl)uracil (prepared according to F. Hansske, D. Madej and M. J. Robins, Tet., 1984, 40, 125) in 5 ml of anhydrous pyridine was treated with 120 μl of acetic anhydride and stirred at room temperature for 30 hours. A further 120 μl of acetic anhydride was added and stirring continued for a further 3 days. The reaction mixture was treated with 0.2 ml of water and then evaporated. The pale yellow oily residue was taken up in 70 ml of dichloromethane and the solution washed with three 10 ml portions of 1M hydrochloric acid then dried over anhydrous sodium sulphate, filtered and evaporated to give 0.53 g of 1-(3-O-acetyl-2,5-bis-O-t-butyldimethylsilyl-β-D-xylofuranosyl)uracil as a pale yellow oil which was used without further purification.

A solution of 0.629 g of 1,2,4-triazole in 15 ml of anhydrous acetonitrile was treated with 182 μl of phosphorus oxychloride. A white suspension formed which was cooled in ice for 15 min then treated with 1.21 ml of triethylamine. The ice bath was removed while a solution of 0.52 g of 1-(3-O-acetyl-2,5-bis-O-t-butyldimethylsilyl-β-D-xylofuranosyl)uracil in 10 ml of dry acetonitrile was added dropwise over 3 minutes. The reaction mixture was stirred at room temperature under nitrogen overnight then diluted with dichloromethane and washed with saturated sodium hydrogen carbonate solution. The dichloromethane solution was dried over anhydrous sodium sulphate, filtered and evaporated. The residue was purified by flash chromatography on silica gel using ethyl acetatelisohexane (1:9 to 3:5) for the elution to give 0.286 g of 1-(3-O-acetyl-2,5-bis-O-tert-butyldimethylsilyl-β-D-ribofuranosyl)-4-(1-triazolyl)pyrimidin-2(1H)-one; mass spectrum(ESI) m/z 566 [M+H]⁺.

A solution of 0.28 g of 1-(3-O-acetyl-2,5-bis-O-t-butyldimethylsilyl-β-D-ribofuranosyl)-4-(1-triazolyl)pyrimidine-2(1H)-one in 10 ml of 1,4-dioxane was treated with 0.5 ml of concentrated aqueous ammonia solution and stirred at room temperature for 12 hours then evaporated to yield 0.25 g of 1-(3-O-acetyl-2,5-bis-O-t-butyldimethylsilyl-β-D-xylofuranosyl)cytosine as a white solid; mass spectrum(ESI) m/z 514 [M+H]⁺.

### Example 248

The compound may be prepared according to H. Hayakawa et al, Chem. Pharm.Bull., 1990, 38(5), 1136. A mixture of 0.3 g of 1-(3-deoxy-3-fluoro-2,5-bis-O-triphenylmethyl-β-D-xylofuranosyl)uracil (prepared according to H. Hayakawa et al, Chem. Pharm. Bull., 1990, 38, 1136) and 80% acetic acid was stirred and heated at 100° C. for 5 hours then evaporated to dryness. The residue was redissolved in 10 ml of distilled water and the solution washed with three 5 ml portions of diethyl ether. The aqueous solution was evaporated to dryness and the residue purified by flash chromatography on silica gel using methanol/dichloromethane (1:19 to 1:12) for the elution to give 53 mg of 1-(3-deoxy-3-fluoro-β-D-xylofuranosyl)uracil; mass spectrum(CI) m/z 246 [M+H]⁺.

### Example 249

The compound may be prepared according to J. A. Wright, D. P. Wilson and J. J. Fox, J. Med. Chem. 1970, 13(2), 269. A solution of 0.2 g of 1-(3-deoxy-3-fluoro-2,5-bis-O-triphenylmethyl-β-D-xylofuranosyl)cytosine in 5 ml of dry methanol was stirred with 1.2 g of Amberlyst 15 ion exchange resin for 5 hours. The resin was filtered off and washed with methanol then suspended in 10 ml of methanol/1M ammonia solution(1:1) and stirred for 30 min. The mixture was filtered and the resin washed thoroughly with methanol. The filtrate was evaporated to a glass which was purified by flash chromatography on silica gel using methanol/dichloromethane (1:4) for the elution to give 13 mg of 1-(3-deoxy-3-fluoro-β-D-xylofuranosyl)cytosine; mass spectrum(ESI) m/z 246 [M+H]⁺.

The 1-(3-deoxy-3-fluoro-2,5-bis-O-triphenylmethyl-β-D-xylofuranosyl)cytosine used as the starting material was prepared as follows:

A solution of 1.71 g of 1,2,4-triazole in 20 ml of anhydrous acetonitrile was stirred under nitrogen and treated with 0.47 ml of phosphorus oxychloride to give a milky suspension which was cooled to <5° C. for 15 min then treated with 3.2 ml of triethylamine. After allowing to warm to room temperature a suspension of 2.0 g of 1-(3-deoxy-3-fluoro-2,5-bis-O-triphenylmethyl-β-D-xylofuranosyl)uracil (prepared according to H. Hayakawa et al, Chem. Pharm. Bull., 1990, 38, 1136) in 15 ml of acetonitrile was added and the mixture stirred at room temperature for 24 hours. The mixture was diluted with dichloromethane and washed with saturated sodium hydrogen carbonate solution. The dichloromethane solution was dried over anhydrous sodium sulphate, filtered and evaporated. The residue was purified by flash chromatography on silica gel using ethyl acetate/isohexane (1:1) for the elution to give 0.5 g of 1-(3-deoxy-3-fluoro-2,5-bis-O-triphenylmethyl-β-D-xylofuranosyl)-4-(1,2,4-triazolyl)pyrimidin-2(1H)-one as a white solid; mass spectrum (ESI) m/z 782 [M+H]⁺.

A solution of 0.5 g of 1-(3-deoxy-3-fluoro-2,5-bis-O-triphenylmethyl-β-D-xylofuranosyl)-4-(1,2,4-triazolyl)pyrimidin-2(1H)-one in 10 ml of 1,4-dioxane was treated with 1 ml of concentrated ammonia solution and stirred at room temperature for 16 hours. The solution was evaporated to dryness and the residue purified by flash chromatography on silica gel using ethyl acetate/isohexane (1:1) for the elution to give 0.23 g of 1-(3-deoxy-3-fluoro-2,5-bis-O-triphenylmethyl-β-D-xylofuranosyl)cytosine; mass spectrum (CI) m/z 731 [M+H]⁺.

### Example 250

The compound may be prepared according to R. Z. Sterzycki, M. M. Mansuri and J. C. Martin, Eur. Pat. Appl. (1990) EP 391411. A solution of 55 mg of 4-N-acetyl-1-(3-acetoxymethyl-2,3-di-O-acetyl-3-deoxy -β-D-ribofuranosyl)cytosine in 0.5 ml of anhydrous methanol was treated with 0.05 ml of 1M sodium methoxide solution and stirred at room temperature for 5 hours. The solution was neutralised by addition of a few drops of glacial acetic acid and evaporated. The residue was purified by recrystallisation from methanol/ethyl acetate to give 3′-deoxy-3′-hydroxymethylcytidine as a white solid; mass spectrum (ESI) m/z 258[M+H]⁺.

The 4-N-acetyl-l1-(3-acetoxymethyl-2,3-di-O-acetyl-3-deoxy-β-D-ribofuranosyl)cytosine used as the starting material was prepared as follows:

A mixture of 0.3 g of 3-acetoxymethyl-1,2,5-tri-O-acetyl-3-deoxy-β-D-ribofuranose (prepared by the procedure of R. M. Sterzycki et al Eur.Pat.Appl. 391411), 0.457 g of N-acetylcytosine and 0.74 ml of bis-trimethylsilylacetamide in 15 ml of anhydrous acetonitrile was heated under reflux for 2.5 hours to give a clear solution. The solution was cooled and treated with 0.28 ml of trimethylsilyl trifluoromethanesulphonate then heated at 50° C. for 3 days. The pale yellow solution was diluted with 100 ml of ethyl acetate and washed with 50 ml of 1M hydrochloric acid, 50 ml of saturated sodium hydrogen carbonate then brine. The solution was dried over anhydrous magnesium sulphate, filtered and evaporated. The residue was purified by flash chromatography on silica gel using methanol/dichloromethane (1:19) for the elution to give 55 mg of 4-N-acetyl-1-(3-acetoxymethyl-2,3-di-O-acetyl-3-deoxy-β-D -ribofuranosyl)cytosine; mass spectrum (ESI) 426[M+H]^{+.}

### Example 251

The compound may be prepared according to R. Z. Sterzycki, M. M. Mansuri and J. C. Martin, Eur. Pat. Appl. (1990) EP 391411. 2′-Deoxy-2′-methoxyuridine is available commercially from ICN Biomedicals Inc., Cat. No. 104991.

### Example 252

The compound may be prepared according to E. Lescrinier et al, Nucleosides and Nucleotides, 1996, 15, 1863. In a manner analogous to that described in Example 38 starting with 6-chloro-9-(β-D-ribofuranosyl)purine was prepared 6-ethylamino-9-(β-D-ribofuranosyl)purine; mass spectrum(ESI) m/z 296 [M+H]⁺.

### Example 253

The compound may be prepared according to E. Lescrinier et al, Nucleosides and Nucleotides, 1996, 15, 1863. In a manner analogous to that described in Example 38 starting with 6-chloro-9-(β-D-ribofuranosyl)purine was prepared 6-propylamino-9-(β-D-ribofuranosyl)purine; mass spectrum(ESI) m/z 310 [M+H]⁺.

It will be understood that references herein to treatment extend to prophylaxis as well as to the treatment of existing conditions, and that the treatment of animals includes the treatment of humans as well as other mammals. Furthermore, treatment of an Hepatitis C Virus (HCV) infection, as used herein, also includes treatment or prophylaxis of a disease or a condition associated with or mediated by Hepatitis C Virus (HCV) infection, or the clinical symptoms thereof.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A method for the treatment of hepatitis C infection comprising administering a therapeutically effective amount of a compound of the formula: 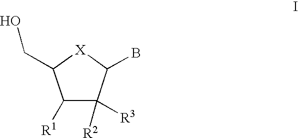
wherein
R¹ is hydrogen, hydroxy, alkyl, hydroxyalkyl, alkoxy, halogen, cyano, isocyano or azido;
R² is hydrogen, hydroxy, alkoxy, chlorine, bromine or iodine;
R³ is hydrogen; or
R² and R³ together represent ═CH₂; or
R² and R³ represent fluorine;
X is O, S or CH₂; and
B is a purine base B1 which is connected through the 9-nitrogen of formula 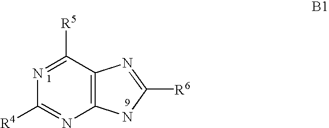
or B is an oxidised purine base B2 which is connected through the 9-nitrogen of formula 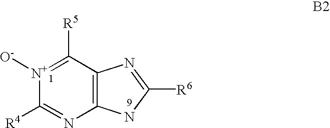
or B is a purine base B3 which is connected through the 9-nitrogen of formula 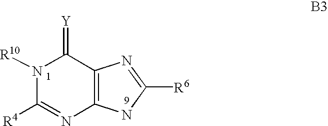
or B is a pyrimidine base B4 which is connected through the 1-nitrogen of formula 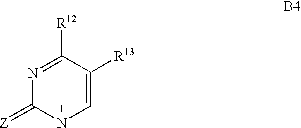
or B is a pyrimidine base B5 which is connected through the 1-nitrogen of formula 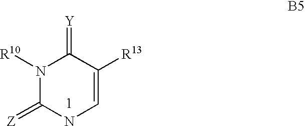
wherein
R⁴ is hydrogen, hydroxy, alkyl, alkoxy, alkylthio, aryloxy, arylthio, heterocyclyl, NR⁷R⁸, halogen or SH;
R⁵ is hydrogen, hydroxy, alkyl, haloalkyl, cycloalkyl, alkoxy, alkylthio, aryl, aryloxy, arylthio, heterocyclyl, heterocyclylamino, halogen, NR⁷R⁸, NHOR⁹, NHNR⁷R⁸ or SH;
R⁶ is hydrogen, hydroxy, alkyl, alkoxy, alkylthio, aryloxy, arylthio, heterocyclyl, NR⁷R⁸, halogen, SH or cyano;
R⁷ and R⁸ are independently of each other hydrogen, alkyl, aryl, hydroxyalkyl, alkenylalkyl, alkynylalkyl, cycloalkyl or acyl;
R⁹ is hydrogen, alkyl or aryl;
R¹⁰ is hydrogen, alkyl or aryl;
Y is O, S or NR¹¹;
R¹¹ is hydrogen, hydroxy, alkyl, OR⁹, heterocyclyl or NR⁷R⁸;
Z is O or S;
R¹² is hydrogen, hydroxy, alkyl, alkoxy, haloalkyl, alkylthio, aryl, aryloxy, arylthio, heterocyclyl, heterocyclylamino, halogen, NR⁷R⁸, NHOR⁹, NHNR⁷R⁸ or SH; and
R¹³ is hydrogen, alkyl, hydroxyalkyl, alkoxyalkyl, haloalkyl, cycloalkyl or halogen and the hydrolyzable esters, hydrolyzable ethers and pharmaceutically acceptable salts thereof.

2. The method according to claim 1 wherein
B is a purine base B1 which is connected through the 9-nitrogen of formula 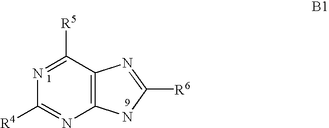
wherein
R⁴ is not NH₂ and R⁵ is not NH(CH₃); or
B is a pyrimidine base B4 which is connected through the 1-nitrogen of formula 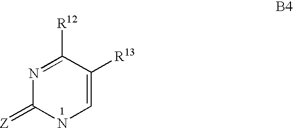
wherein
R¹² is not hydroxy, alkoxy, N(CH₃)₂, N(H)NH(CH₃) or N(H)NH₂ and R¹³ is not hydroxyalkyl, chlorine or bromine; or
B is a pyrimidine base B5 which is connected through the 1-nitrogen of formula 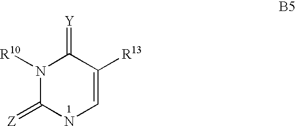
wherein
R¹⁰ is not methyl or hydroxyethyl.

3. The method according to claim 1 wherein
R¹ is hydrogen, hydroxy, alkyl, hydroxyalkyl, alkoxy, halogen;
R² is hydrogen, hydroxy, alkoxy, chlorine, bromine or iodine;
R³ is hydrogen; or
R² and R³ are fluorine; and
X is O.

4. The method according to claim 1 wherein
R¹ is hydroxy;
R² is hydroxy;
R³ is hydrogen; and
X is O.

5. The method according to claim 1 wherein
B is a purine base B1 which is connected through the 9-nitrogen of formula 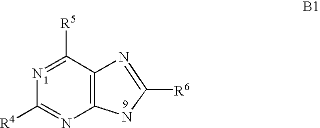
wherein
R⁴ is hydrogen, hydroxy, alkyl, alkoxy, alkylthio, aryloxy, arylthio, heterocyclyl, NR⁷R⁸, halogen or SH;
R⁵ is hydrogen, hydroxy, alkyl, haloalkyl, cycloalkyl, alkoxy, alkylthio, aryl, aryloxy, arylthio, heterocyclyl, heterocyclylamino, halogen, NR⁷R⁸, NHOR⁹, NHNR⁷R⁸ or SH;
R⁶ is hydrogen, hydroxy, alkyl, alkoxy, alkylthio, aryloxy, arylthio, heterocyclyl, NR⁷R⁸, halogen, SH or cyano;
R⁷ and R⁸ are independently of each other hydrogen, alkyl, aryl, hydroxyalkyl, alkenylalkyl, alkynylalkyl, cycloalkyl or acyl;
R⁹ is hydrogen, alkyl or aryl.

6. The method according to claim 1 wherein
B is a purine base B1 which is connected through the 9-nitrogen of formula 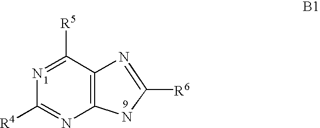
wherein
R⁴ is hydrogen, chlorine or NH₂;
R⁵ is hydroxy, alkylthio, aryl, heterocyclyl, halogen, NR⁷R⁸ or SH;
R⁶ is hydrogen, halogen, heterocyclyl or NR⁷R⁸;
R⁷ and R⁸ are independently of each other hydrogen, alkyl, aryl, alkenylalkyl or alkynylalkyl.

7. The method according to claim 1 wherein
B is a purine base B1 which is connected through the 9-nitrogen of formula 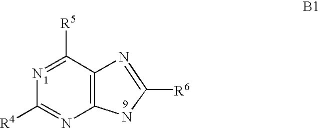
wherein
R⁴ is hydrogen;
R⁵ is alkylthio, aryl, heterocyclyl, halogen or NR⁷R⁸;
R⁶ is hydrogen or halogen;
R⁷ and R⁸ are independently of each other hydrogen, alkyl, alkenylalkyl or alkynylalkyl.

8. The method according to claim 2 wherein
B is the purine base B1.

9. The method according to claim 8 wherein
R⁴ is hydrogen or chlorine;
R⁵ is hydroxy, alkylthio, aryl, heterocyclyl, halogen, NR⁷R⁸ or SH;
R⁶ is hydrogen, halogen, heterocyclyl or NR⁷R⁸;
R⁷ and R⁸ are independently of each other hydrogen, alkyl, aryl, alkenylalkyl or alkynylalkyl.

10. The method according to claim 9 wherein
R⁴ is hydrogen;
R⁵ is alkylthio, aryl, heterocyclyl, halogen or NR⁷R⁸;
R⁶ is hydrogen or halogen;
R⁷ and R⁸ are independently of each other hydrogen, alkyl, alkenylalkyl or alkynylalkyl.

11. The method according to claim 1 wherein the compound is selected from the group consisting of:
6-Dimethylamino-9-(β-D-ribofuranosyl)purine,
6-[1(S)-Methyl-2-phenylethylamino]-9-(β-D-ribofuranosyl)purine,
3′-Deoxyadenosine,
6-(Phenylethylamino)-9-(β-D-ribofuranosyl)purine,
6-(Cyclohexylamino)-9-(β-D-ribofuranosyl)purine,
2-Chloroadenosine,
9-(β-D-Ribofuranosyl)purine,
8-Bromoadenosine,
8-Bromo-2′-deoxyadenosine,
8-Bromoguanosine,
6-Thioinosine,
6-Methylthio-9-(β-D-ribofuranosyl)purine,
6-Chloro-9-(β-D-ribofuranosyl)purine,
2-Amino-6-chloro-9-(β-D-ribofuranosyl)purine,
6-(N-Methylpropylamino)-9-(β-D-ribofuranosyl)purine,
9-(β-D-Ribofuranosyl)-6-(4-thiomorpholinyl)purine,
6-(N-Methyl-2-propenylamino)-9-(β-D-ribofuranosyl)purine,
6-(N-Methyl-2-propynylamino)-9-(β-D-ribofuranosyl)purine,
6-(4-Morpholinyl)-9-(β-D-ribofuranosyl)purine,
6-Diethylamino-9-(β-D-ribofuranosyl)purine,
6-(1(R,S)-Phenylethylamino)-9-(β-D-ribofuranosyl)purine,
6-(1-Benzyl-1-methylethylamino)-9-(β-D-ribofuranosyl)purine,
6-(3-Phenylpropylamino)-9-(β-D-ribofuranosyl)purine,
9-(β-D-Ribofuranosyl)-6-[2-(2-thienyl)ethylamino]purine,
6-Dibenzylamino-9-(β-D-ribofuranosyl)purine,
6-Hexylamino-9-(β-D-ribofuranosyl)purine,
6-(3-Pyridylmethylamino)-9-(β-D-ribofuranosyl)purine,
6-[4-(4-Fluorophenyl)-1,2,5,6-tetrahydropyridyl]-9-(β-D-ribofuranosyl)purine,
6-[4-(2-Methoxyphenyl)piperazinyl]-9-(β-D-ribofuranosyl)purine,
6-[2-(3-Indolyl)ethylamino]-9-(β-D-ribofuranosyl)purine,
6-[2-(4-Chlorophenyl)ethylamino)]-9-(β-D-ribofuranosyl)purine,
6-(N-Methylphenylamino)-9-(β-D-ribofuranosyl)purine,
9-(β-D-Ribofuranosyl)-6-(1,2,4,5-tetrahydro-3H-benzazepin-3-yl)purine,
9-(β-D-Ribofuranosyl)-6-(1,2,3,4-tetrahydro-2-isoquinolyl)purine,
6-(4-Methylpiperazinyl)-9-(β-D-ribofuranosyl)purine,
9-(β-D-Ribofuranosyl)-6-(1,3,4,5-tetrahydro-2H-benzazepin-2-yl)purine,
6-[2-(4-Cyanomethylphenyl)ethylamino]-9-(β-D-ribofuranosyl)purine,
6-(2,3-Dihydro-1-indolyl)-9-(β-D-ribofuranosyl)purine,
9-(β-D-Ribofuranosyl)-6-(2,3,4,5-tetrahydro-1,4-benzothiazepin-4-yl)purine,
9-(β-D-Ribofuranosyl)-6-(2,3,4,5-tetrahydro-1,4-benzoxazepin-4-yl)purine,
6-(8-Aminosulphonyl-2,3,4,5-tetrahydro-1H-2-benzazepin-2-yl)-9-(β-D-ribofuranosyl)purine,
6-[2-(3,4-Dimethoxyphenyl)ethylamino)-9-(β-D-ribofuranosyl)purine,
6-[-2-(4-Hydroxyphenyl)ethylamino]-9-(β-D-ribofuranosyl)purine,
6-(2-Isoindolinyl)-9-(β-D-ribofuranosyl)purine,
6-(7-Aminosulphonyl-2,3,4,5-tetrahydro-1H-benzazepin-3-yl)-9-(β-D-ribofuranosyl)purine,
6-(N-Cyclohexylmethylamino)-9-(β-D-ribofuranosyl)purine,
6-(N-Hexylmethylamino)-9-(β-D-ribofuranosyl)purine,
6-(10,11-Dihydro-5H-dibenzo[a,d]cyclohepten-5-ylamino)-9-(β-D-ribofuranosyl)purine,
6-[N-(10,11-Dihydro-5H-dibenzo[a,d]cyclohepten-5-yl)methylamino]-9-(β-D-ribofuranosyl)purine,
6-[N-(5-Aminopentyl)methylamino]-9-(β-D-ribofuranosyl)purine,
6-[(5-Chloro-2-methoxyphenyl)methylamino]-9-(β-D-ribofuranosyl)purine,
6-[(2-Methylphenyl)methylamino]-9-(β-D-ribofranosyl)purine,
6-(Hexamethyleneimino)-9-(β-D-ribofuranosyl)purine,
6-(1-Pyrrolidinyl)-9-(β-D-ribofuranosyl)purine,
6-(4-Hydroxypiperidin-1-yl)-9-(β-D-ribofuranosyl)purine,
6-(1-Piperidinyl)-9-(β-D-ribofuranosyl)purine,
6-(2-Propenyl)amino-9-(β-D-ribofuranosyl)purine,
6-(2-Propynyl)amino-9-(β-D-ribofuranosyl)purine,
6-(1-Methyl)ethylamino-9-(β-D-ribofuranosyl)purine,
6-bis-(2-Propenyl)amino-9-(β-D-ribofuranosyl)purine,
6-(2-Phenylethyl)methylamino-9-(β-D-ribofuranosyl)purine,
6-Ethylmethylamino-9-(β-D-ribofuranosyl)purine,
6-bis-[(3-Methyl)butylamino]-9-(β-D-ribofuranosyl)purine,
6-(4-Aminophenyl)methylamino-9-(β-D-ribofuranosyl)purine,
6-(2-Pyridylmethyl)amino-9-(β-D-ribofuranosyl)purine,
6-(2-Hydroxyethyl)methylamino-9-(β-D-ribofuranosyl)purine,
6-Dipropylamino-9-(β-D-ribofuranosyl)purine,
6-[2-Phenyl-(N-propionyl)ethylamino]-9-(β-D-ribofuranosyl)purine,
6-(N-Benzoyl-2-phenylethylamino)-9-(β-D-ribofuranosyl)purine,
2-Amino-6-methylamino-9-(β-L-ribofuranosyl)purine,
2-Amino-6-methylamino-9-(β-D-ribofuranosyl)purine,
2-Amino-6-(4-morpholinyl)-9-(β-D-ribofuranosyl)purine,
2-Amino-6-(1-pyrrolidinyl)-9-(β-D-ribofuranosyl)purine,
2,6-Diamino-9-(β-L-ribofuranosyl)purine,
2,6-Diamino-9-(β-D-ribofuranosyl)purine,
2-Chloro-6-(1-pyrrolidinyl)-9-(β-D-ribofuranosyl)purine,
2-Chloro-6-(1-hexamethyleneimino)-9-(β-D-ribofuranosyl)purine,
2-Chloro-6-(4-hydroxy-1-piperidinyl)-9-(β-D-ribofuranosyl)purine,
6-[(N-Cyclohexyl)methylamino]-2-methylthio-9-(β-D-ribofuranosyl)purine,
6-(1-Pyrrolyl)-9-(β-D-ribofuranosyl)purine,
6-(1-Pyrrolyl)-9-(β-D-ribofuranosyl)purine,
6-(1-Pyrrolyl)-9-(β-D-ribofuranosyl)purin-8-(7H)-one,
9-(3-Deoxy-β-D-ribofuranosyl)-6-(1-pyrrolyl)purine,
6-(1-Pyrrolyl)-9-(β1-L-ribofuranosyl)purine,
6-(1-Indolyl)-9-(β-D-ribofuranosyl)purine,
6-(1-Imidazolyl)-9-(β-D-ribofuranosyl)purine,
9-(β-D-Ribofuranosyl)-6-(1,2,4-triazol-1-yl)purine,
6-(1-Pyrazolyl)-9-(β-D-ribofuranosyl)purine,
9-(β-D-Ribofuranosyl)6-(1,2,4-triazol-4-yl)purine,
6-Methylamino-9-(β-D-ribofuranosyl)purin-2(1H)-one,
2-Methoxy-6-methylamino-9-(β-D-ribofuranosyl)purine,
2-Methoxyadenosine,
2,6-Dichloro-9-(β-D-ribofuranosyl)purine,
6-Methoxy-9-(β-D-ribofuranosyl)purine,
2-Amino-6-benzylthio-9-(β-D-ribofuranosyl)purine,
6-Benzylthio-2-hydroxy-9-(β-D-ribofuranosyl)purine,
9-(β-D-Ribofuranosyl)purine-2,6,8(1H,3H,7H)-trione,
8-(Methylamino)adenosine,
8-(2-Phenylethylamino)adenosine,
8-Benzylaminoadenosine,
8-(1-Piperidinyl)adenosine,
8-(Dimethylamino)adenosine,
8-(3-Phenylpropylamino)adenosine,
8-(4-Morpholinyl)adenosine,
8-(N-Methyl-2-phenylethylamino)adenosine,
8-(3-Pyridylmethylamino)adenosine,
8-(Ethylamino)adenosine,
8-(1,2,3,4-Tetrahydro-2-isoquinolyl)adenosine,
8-[2-(4-Morpholinyl)ethylamino]adenosine,
8-(Hexylamino)adenosine,
8-(2-Cyclohexylethylamino)adenosine,
8-(2(R,S)-Phenylpropylamino)adenosine,
8-[2-(4-Methylphenyl)ethylamino]adenosine,
8-[2-(1-methyl-2-pyrrolyl)ethylamino]adenosine,
8-[2-(4-Aminosulphonylphenyl)ethylamino]adenosine,
8-(4-Phenyl-1-piperazinyl)adenosine,
8-(2-(4-Imidazolyl)adenosine,
8-(1-Naphthylmethylamino)adenosine,
8-[2-(4-Hydroxyphenyl)ethylamino]adenosine,
8-(4-Phenylbutylamino)adenosine,
8-[2-(4-Chlorophenyl)ethylamino]adenosine,
8-[2-(2,4-Dichlorophenyl)ethylamino]adenosine,
8-(2-Propenylamino)adenosine,
8-(2-Hydroxyethylamino)adenosine,
8-(1(R)-Methyl-2-phenylethylamino)adenosine,
8-(4-Fluorobenzylamino)adenosine,
8-[(4-Hydroxycarbonyl)benzylamino]adenosine,
8-(2-Propynylamino)adenosine,
8-(1-Methylethylamino)adenosine,
8-[(4-Trifluoromethyl)benzylamino]adenosine,
8-[(2,5-Dimethoxy)benzylamino]adenosine,
8-[2-(2-Thienyl)ethylamino]adenosine,
8-[2-(4-Aminophenyl)ethylamino]adenosine,
8-(2-Phenoxyethylamino)adenosine,
8-[(2-Thienyl)methylamino)adenosine,
8-[(4-tert-Butyl)benzylamino]adenosine,
8-(1(R)-Phenylethylamino)adenosine,
8-(1(S)-Phenylethylamino)adenosine,
8-(6-Phenylhexylamino)adenosine,
8-[2-Hydroxy-1(S)-phenyl)ethylamino]adenosine,
2′-Deoxy-8-(2-phenylethylamino)adenosine,
2′-Deoxy-8-(3-phenylpropylamino)adenosine,
8-Benzylamino-2′-deoxyadenosine,
2′-Deoxy-8-(4-phenylbutylamino)adenosine,
2′-Deoxy-8-(6-phenylhexylamino)adenosine,
8-(4-Morpholinyl)inosine,
8-(Methylthio)adenosine,
8-(Benzylthio)adenosine,
8-(Benzyloxy)adenosine,
8-Ethoxyadenosine,
8-[(1-Hydroxy-1-methyl)ethyl]adenosine,
9-(β-D-ribofuranosyl)-6-(3-thienyl)purine,
6-Phenyl-9-(β-D-ribofuranosyl)purine,
6-(4-Fluorophenyl)-9-(β-D-ribofuranosyl)purine,
6-(4-Chlorophenyl)-9-(β-D-ribofuranosyl)purine,
6-(4-Methylphenyl)-9-(β-D-ribofuranosyl)purine,
6-(4-Methoxyphenyl)-9-(β-D-ribofuranosyl)purine,
9-(β-D-Ribofuranosyl)-6-(1-thianthrenyl)purine,
6-(4-Biphenylyl)-9-(β-D-ribofuranosyl)purine,
6-(4-Methylthiophenyl)-9-(β-D-ribofuranosyl)purine,
6-(2-Methylphenyl)-9-(β-D-ribofuranosyl)purine,
6-(9-Phenanthrenyl)-9-(β-D-ribofuranosyl)purine,
9-(β-D-Ribofuranosyl)-6-(3-trifluoromethylphenyl)purine,
6-(2-Phenoxyphenyl)-9-(β-D-ribofuranosyl)purine,
6-(4-tert-Butylphenyl)-9-(β-D-ribofuranosyl)purine,
9-(β-D-Ribofuranosyl)-6-(2-trifluoromethoxyphenyl)purine,
6-(4-Phenoxyphenyl)-9-(β-D-ribofuranosyl)purine,
6-(3-Methoxyphenyl)-9-(β-D-ribofuranosyl)purine,
6-(2-Naphthyl)-9-(β-D-ribofuranosyl)purine,
6-(3-Biphenylyl)-9-(β-D-ribofuranosyl)purine,
6-[4-(2-Methylpropyl)phenyl]-9-(β-D-ribofuranosyl)purine,
6-(3-Fluorophenyl)-9-(β-D-ribofuranosyl)purine,
9-(β-D-Ribofuranosyl)-6-(4-trifluoromethylphenyl)purine,
6-(3-Ethoxyphenyl)-9-(β-D-ribofuranosyl)purine,
6-[3-(1-Methyl)ethylphenyl]-9-(β-D-ribofuranosyl)purine,
9-(β-D-ribofuranosyl)-6-(4-trifluoromethoxyphenyl)purine,
6-(4-Ethylphenyl)-9-(β-D-ribofuranosyl)purine,
2-Amino-6-phenyl-9-(β-D-ribofuranosyl)purine,
6-Ethylamino-9-(β-D-ribofuranosyl)purine, and
6-Propylamino-9-(β-D-ribofuranosyl)purine.

12. The method according to claim 1 wherein B is the oxidized purine base B2.

13. The method according to claim 12 wherein
R⁴ is hydrogen;
R⁵ is hydrogen, alkyl, heterocyclyl or NR⁷R⁸;
R⁶ is hydrogen;
R⁷ and R⁸ are independently of each other hydrogen, alkyl, aryl, hydroxyalkyl, alkenylalkyl, alkynylalkyl, cycloalkyl or acyl.

14. The compound according to claim 13 which compound is
Adenosine-1-oxide, or
6-(2-Phenylethylamino)-9-(β-D-ribofuranosyl)purine-1-oxide.

15. The method according to claim 1 wherein B is the purine base B3.

16. The method according to claim 15 wherein
R⁴ is hydrogen, NR⁷R⁸ or hydroxy;
R⁶ is hydrogen, halogen or NR⁷R⁸;
R⁷ and R⁸ are independently of each other hydrogen or alkyl;
R¹⁰ is hydrogen or alkyl;
Y is O, S, NH or N-alkyl.

17. The method according to claim 1 wherein the compound is
3′-Deoxyguanosine,
6-Thioguanosine,
Inosine,
L-Inosine,
8-Bromoinosine,
1-Benzyl-6-imino-9-(β-D-ribofuranosyl)purine,
1-Methyl-6-(2-phenylethylimino)-9-(β-D-ribofuranosyl)purine,
2-(Acetylamino)inosine, or
8-(Benzylamino)inosine.

18. The method according to claim 1 wherein B is the pyrimidine base B4.

19. The method according to claim 18 wherein
Z is O;
R¹² is hydroxy, alkyl, heterocyclyl, NR⁷R⁸, NHOR⁹, heterocyclylamino, NHNR⁷R⁸ or SH; and
R¹³ is hydrogen, alkyl or halogen.

20. The method according to claim 19 wherein
R¹² is hydroxy, alkyl or NR⁷R⁸;
R¹³ is hydrogen;
R⁷ and R⁸ are independently of each other hydrogen or alkyl.

21. The method according to claim 1 wherein
R¹ is hydrogen, halogen, hydroxy, alkyl, alkoxy, cyano or azido;
R² is hydrogen or hydroxy; or
R² and R³ represent fluorine;
X is O or CH₂;
B is the pyrimidine base B4;
Z is O;
R¹² is NR⁷R⁸;
R¹³ is hydrogen, alkyl or halogen;
R⁷ and R⁸ are independently of each other hydrogen or alkyl.

22. The method according to 21 wherein
R¹³ is hydrogen, C₁₋₄-alkyl or fluorine;
R⁷ and R⁸ are independently of each other hydrogen or C₁₋₄-alkyl.

23. The method according to claim 2 wherein
B is the pyrimidine base B4;
R¹² is hydrogen, alkyl, haloalkyl, alkylthio, aryl, aryloxy, arylthio, heterocyclyl, heterocyclylamino, halogen, NR⁷R⁸, NHOR⁹, NHNR⁷R⁸ or SH;

24. The method according to claim 23 wherein
Z is O;
R¹² is alkyl, heterocyclyl, NR⁷R⁸, NHOR⁹, heterocyclylamino, NHNR⁷R⁸ or SH; and
R¹³ is hydrogen, alkyl or halogen.

25. The method according to claim 24 wherein
R¹² is alkyl or NR⁷R⁸;
R¹³ is hydrogen; and
R⁷ and R⁸ are independently of each other hydrogen or alkyl.

26. The method according to claim 1 wherein the compound is
4-Thiouridine,
5-Fluorocytidine,
1-(β-D -arabinofuranosyl)-5-fluorocytosine,
5-Methylcytidine,
2′,3′-Dideoxycytidine,
N4-Acetylcytidine,
3′-Deoxycytidine,
4-Methoxy-1-(β-D-ribofuranosyl)pyrimidin-2(1H)-one,
4-Methylthio-1-(β-D-ribofuranosyl)pyrimidin-2(1H)-one,
5-Fluoro-4-methylthio-1-(β-D-ribofuranosyl)pyrimidin-2(1H)-one,
5-Methyl-4-methylthio-1-(β-D-ribofuranosyl)pyrimidin-2(1H)-one,
3′-Azido-2′,3′-dideoxy-5-methylcytidine,
1-(3-Deoxy-β-L-threo-pentofuranosyl)-5-fluorocytosine,
4-Methylamino-1-(β-D-ribofuranosyl)pyrimidin-2(1H)-one,
5-Fluoro-4-methylamino-1-(β-D-ribofuranosyl)pyrimidin-2(1H)-one,
4-(1-Pyrrolyl)-1-(β-D-ribofuranosyl)pyrimidin-2(1H)-one,
1-(2-Deoxy-2,2-difluoro-β-D-erythropentofuranosyl)cytosine,
4-Amino-1(R)-(2(S),3(R)-dihydroxy-4(R)-hydroxymethyl-cyclopentyl)-1H-pyrimidin-2-one,
1-(β-D-Xylofuranosyl)cytosine,
1 (3-Deoxy-3-fluoro-β-D-xylofuranosyl)cytosine, or
3′-Deoxy-3′-hydroxymethylcytidine.

27. The method according to claim 2 wherein
R¹ is hydrogen, halogen, hydroxy, alkyl, alkoxy, cyano or azido;
R² is hydrogen or hydroxy; or
R² and R³ represent fluorine;
X is O or CH₂;
B is the pyrimidine base B4;
Z is O;
R¹² is NR⁷R⁸;
R¹³ is hydrogen, alkyl or halogen; and
R⁷ and R⁸ are independently of each other hydrogen or alkyl;

28. The method according to claim 27 wherein
R¹ is hydrogen, fluorine, hydroxy, C₁₋₄-alkyl, C₁₋₄-alkoxy, cyano or azido;
R¹³ is hydrogen, C₁₋₄-alkyl or fluorine; and
R⁷ and R⁸ are independently of each other hydrogen or C₁₋₄-alkyl.

29. The method according to claim 28 wherein the compound is
L-Cytidine, or
4-Amino-1-(2,2-difluoro-3-hydroxy-4-hydroxymethyl-cyclopentyl)-1H-pyrimidin-2-one.

30. The method according to claim 1, wherein B is the pyrimidine base B5.

31. The method according to claim 30 wherein
wherein
Y is O or NR¹¹;
Z is O;
R¹⁰ is hydrogen; and
R¹³ is hydrogen, alkyl or halogen.

32. The method according to claim 2 wherein B is the pyrimidine base B5.

33. The method according to claim 1 wherein the compound is
2′-Deoxy-5-fluorouridine,
1-(β-D-Arabinofuranosyl)-5-fluorouracil,
5-Fluorouridine,
5-Bromouridine,
3-Methyluridine,
5-Methyluridine,
1-(β-D-Arabinofuranosyl)uracil,
1-(β-D-Arabinofuranosyl)-5-methyluracil,
1-(β-D-Arabinofuranosyl)-5-iodouracil,
3′-Deoxy-5-methyluridine,
5-Ethyluridine,
5-[(1-Methyl)ethyl]uridine,
5-Methoxymethyluridine,
5-Ethoxymethyluridine,
5-Chlorouridine,
5-Methyl-1-(β-L-ribofuranosyl)uracil,
1-(β-D-Arabinofuranosyl)-5-ethyluracil,
1-(β-D-Arabinofuranosyl)-5-bromo uracil,
5-Methyl-4-thiouridine,
5-Fluoro-4-thiouridine,
1-(2-Deoxy-α-D-erthyro-pentofuranosyl)-5-fluorouracil,
2′-Deoxy-5-fluoro-3-methyluridine,
1-(α-D-Erthyro-2-deoxypentofuranosyl)-5-fluoro-3-methyluracil,
2′-Chloro-2′-deoxyuridine,
2′-Bromo-2′-deoxyuridine,
1-(2-Deoxy-β-D-lyxofuranosyl)-5-methyluracil,
3′-Deoxy-3′-fluoro-5-methyluridine,
2′,3′-Dideoxy-5-ethyl-3′-methoxyuridine,
5′-Benzyloxy-2′,3′-dideoxy-5-methyluridine,
2′,3′-Dideoxy-5-ethyl-3′-iodouridine,
3′-Azido-2′,3′-dideoxy-5-ethyluridine,
4-Oximino-1-(β-L-ribofuranosyl)pyrimidin-2(1H)-one,
4-Oximino-1-(β-D-ribofuranosyl)pyrimidin-2(1H)-one,
4-Oximino-1-(β-D-arabinofuranosyl)pyrimidin-2(1H)-one,
5-Fluoro-4-oximino-1-(β-D-ribofuranosyl)pyrimidin-2(1H)-one,
1-(2-Deoxy-2,2-difluoro-α-D-erythropentofuranosyl)uracil,
1-(3-Deoxy-3-fluoro-β-D-xylofuranosyl)uracil, or
2′-Deoxy-2′-methoxyuridine.

34. A compound of the formula I-a 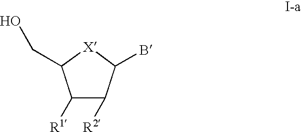
wherein
R¹′ is hydroxy;
R²′ is hydroxy;
X′ is O;
B′ is an oxidized purine base B2-a which is connected through the 9-nitrogen of formula 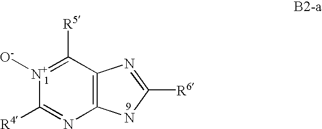
wherein
R⁴′ is hydrogen;
R⁵′ is NHR⁸′;
R⁶′ is hydrogen;
R⁸′ is alkyl;
and the hydrolyzable esters, hydrolyzable ethers, and pharmaceutically acceptable salts thereof.

35. The compound according to claim 34 which is:
6-(2-phenylethylamino)-9-(β-D-ribofuranosyl)purine-1-oxide.

36. A compound of the formula I-b 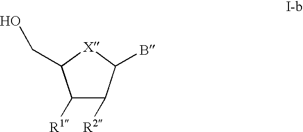
wherein
R¹″ is hydroxy;
R²″ is hydroxy;
X″ is O;
B″ is a purine base B3-a which is connected through the 9-nitrogen of formula 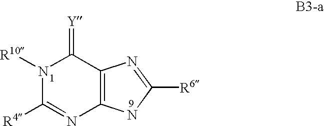
wherein
R⁴″ is hydrogen;
R⁶″ is hydrogen;
R¹⁰″ is alkyl;
Y″ is NR¹¹″;
R¹¹″ is alkyl;
and the hydrolyzable esters, hydrolyzable ethers, and pharmaceutically acceptable salts thereof.

37. The compound according to claim 36 which is:
1-Methyl-6-(2-phenylethylimino)-9-(β-D-ribofuranosyl)purine.

38. A compound of the formula I-c 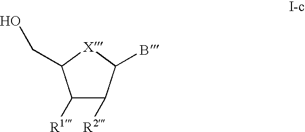
wherein
R¹′″ is hydroxy;
R²′″ is hydroxy;
X′″ is O;
B′″ is a pyrimidine base B4-a which is connected through the 1-nitrogen of formula 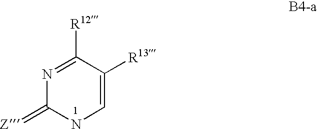
wherein
R¹²′″ is alkylthio or heterocyclyl;
R¹³′″ is hydrogen, alkyl or halogen;
Z′″ is O;
and the hydrolyzable esters or ethers thereof and pharmaceutically acceptable salts thereof.

39. The compound according to claim 38 which compound is
5-Fluoro-4-methylthio-1-(β-D-ribofuranosyl)pyrimidin-2(1H)-one,
5-Methyl-4-methylthio-1-(β-D-ribofuranosyl)pyrimidin-2(1H)-one, or
4-(1-Pyrrolyl)-1-(β-D-ribofuranosyl)pyrimidin-2(1H)-one.

40. A compound of the formula I-d 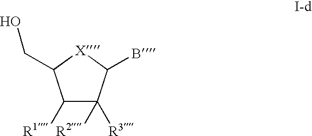
wherein
R¹″″ is hydrogen, halogen, hydroxy, alkyl, alkoxy, cyano or azido;
R²″″ and R³″″ represent fluorine;
X″″ is O or CH₂;
B″″ is a pyrimidine base B4-b which is connected through the 1-nitrogen of formula 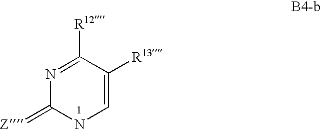
wherein
Z″″ is O;
R¹²″″ is NR⁷″″ R⁸″″;
R¹³″″ is hydrogen, alkyl or halogen;
R⁷″″ and R⁸″″ are independently of each other hydrogen or alkyl;
and the hydrolyzable esters, hydrolyzable ethers, and pharmaceutically acceptable salts thereof.

41. The compound according to claim 40 wherein
R¹″″ is hydrogen, fluorine, hydroxy, C₁₋₄-alkyl, C₁₋₄-alkoxy, cyano or azido;
X″″ is CH₂;
R¹²″″ is hydrogen, C₁₋₄-alkyl or fluorine; and
R⁷″″ and R⁸″″ are independently of each other hydrogen or C₁₋₄-alkyl.

42. The compound according to claim 41 which compound is
4-Amino-1-(2,2-difluoro-3-hydroxy-4-hydroxymethyl-cyclopentyl)-1H-pyrimidin-2-one.

43. A compound of the formula I-e 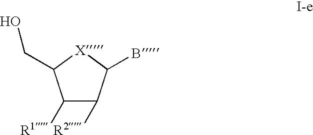
wherein
R¹′″″ is alkoxy;
R²′″″ is hydrogen;
X′″″ is O;
B′″″ is a pyrimidine base B5-a which is connected through the 1-nitrogen of formula 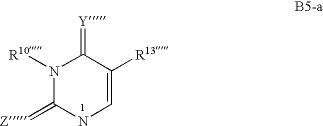
wherein
R¹⁰′″″ is hydrogen;
R¹³′″″ is alkyl;
Y′″″ is O;
Z′″″ is O;
and the hydrolyzable esters, hydrolyzable ethers, and pharmaceutically acceptable salts thereof.

44. The compound according to claim 43 which is
2′,3′-Dideoxy-5-ethyl-3′-methoxyuridine.

45. A compound of the formula I-f 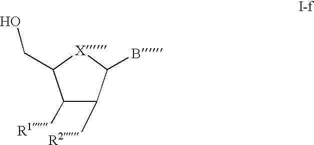
wherein
R¹″″″ is hydroxy;
R²″″″ is hydroxy;
X″″″ is O;
B″″″ is a pyrimidine base B5-b which is connected through the 1-nitrogen of formula 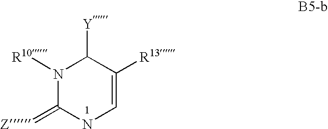
wherein
R¹⁰″″″ is hydrogen;
R¹³″″″ is halogen;
Y″″″ is NR¹¹″″″;
R¹¹″″″ is hydroxy;
Z″″″ is O;
and the hydrolyzable esters, hydrolyzable ethers, and pharmaceutically acceptable salts thereof.

46. The compound according to claim 45 which is
5-Fluoro-4-oximino-1-(β-D-ribofuranosyl)pyrimidin-2(1H)-one.

47. A compound of the formula I-g 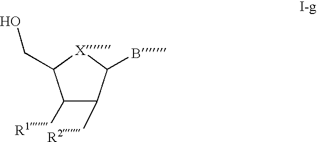
wherein
R¹′″″″ hydroxy;
R²′″″ is hydroxy;
B′″″″ is a pyrimidine base B5-c which is connected through the 1-nitrogen of formula 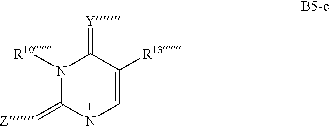
wherein
R¹⁰′″″″ is hydrogen;
R¹³′″″″ is hydrogen;
Y′″″″ is NR¹¹′″″″;
R¹¹′″″″ is hydroxy;
Z′″″″ is O;
and the hydrolyzable esters, hydrolyzable ethers and pharmaceutically acceptable salts thereof.

48. The compound according to claim 47 which is
4-Oximino-1-(β-L-ribofuranosyl)pyrimidin-2(1H)-one.

49. A compound selected from the group consisting of:
6-(N-Methylpropylamino)-9-(β-D-ribofuranosyl)purine,
9-(β-D-Ribofuranosyl)-6-(4-thiomorpholinyl)purine,
6-(N-(2-Propenyl)methylamino)-9-(β-D-ribofuranosyl)purine,
6-(N-Methyl-2-propynylamino)-9-(β-D-ribofuranosyl)purine,
6-[4-(4-Fluorophenyl)-1,2,5,6-tetrahydropyridyl]-9-(β-D-ribofuranosyl)purine,
6-[4-(2-Methoxyphenyl)piperazinyl]-9-(β-D-ribofuranosyl)purine,
6-(N-Methylphenylamino)-9-(β-D-ribofuranosyl)purine,
9-(β-D-Ribofuranosyl)-6-(1,2,4,5-6-(1-itetrahydro-3H-benzazepin-3-yl)purine,
9-(β-D-ribofuranosyl)-6-(1,2,3,4-tetrahydro-2-isoquinolyl)purine,
9-(β-D-Ribofuranosyl)-6-(1,3,4,5-tetrahydro-2H-benzazepin-2-yl)purine,
6-[2-(4-Cyanomethylphenyl)ethylamino]-9-(β-D-ribofuranosyl)purine,
6-(2,3-Dihydro-1-indolyl)-9-(β-D-ribofuranosyl)purine,
9-(β-D-Ribofuranosyl)-6-(2,3,4,5-tetrahydro-1,4-benzothiazepin-4-yl)purine,
9-(β-D-Ribofuranosyl)-6-(2,3,4,5-tetrahydro-1,4-benzoxazepin-4-yl)purine,
6-(8-Aminosulphonyl-2,3,4,5-tetrahydro-1H-2-benzazepin-2-yl)-9-(β-D-ribofuranosyl)purine,
6-(2-Isoindolinyl)-9-(β-D-ribofuranosyl)purine,
6-(7-Aminosulphonyl-2,3,4,5-tetrahydro-1H-benzazepin-3-yl)-9-(β-D-ribofuranosyl)purine,
6-(10,11-Dihydro-5H-dibenzo[a,d]cyclohepten-5-ylamino)-9-(β-D-ribofuranosyl)purine,
6-[N-(10,11-Dihydro-5H-dibenzo[a,d]cyclohepten-5-yl)methylamino]-9-(β-D-ribofuranosyl)purine,
6-[N-(5-Aminopentyl)methylamino]-9-(β-D-ribofuranosyl)purine,
6-Ethylmethylamino-9-(β-D-ribofuranosyl)purine,
6-bis-[(3-Methyl)butylamino]-9-(β-D-ribofuranosyl)purine,
6-[2-Phenyl-(N-propionyl)ethylamino]-9-(β-D-ribofuranosyl)purine,
6-(N-Benzoyl-2-phenylethylamino)-9-(β-D-ribofuranosyl)purine,
1-Methyl-6-(2-phenylethylimino)-9-(β-D-ribofuranosyl)purine,
2-Amino-6-methylamino-9-(β-L-ribofuranosyl)purine,
6-[(N-Cyclohexyl)methylamino]-2-methylthio-9-(β-D-ribofuranosyl)purine,
6-(1-Pyrrolyl)-9-(β-D-ribofuranosyl)purin-8-(7H)-one,
9-(3-Deoxy-β-D-ribofuranosyl)-6-(1-pyrrolyl)purine,
6-(1-Pyrrolyl)-9-(β-L-ribofuranosyl)purine,
6-(1-Indolyl)-9-(β-D-ribofuranosyl)purine,
6-(1-Imidazolyl)-9-(β-D-ribofuranosyl)purine,
9-(β-D-Ribofuranosyl)-6-(1,2,4-triazol-1-yl)purine,
6-(1-Pyrazolyl)-9-(β-D-ribofuranosyl)purine,
6-(2-Phenylethylamino)-9-(β-D-ribofuranosyl)purine-1-oxide,
8-(2-Phenylethylamino)adenosine,
8-(3-Phenylpropylamino)adenosine,
8-(4-Morpholinyl)adenosine,
8-(N-Methyl-2-phenylethylamino)adenosine,
8-(3-Pyridylmethylamino)adenosine,
8-(1,2,3,4-Tetrahydro-2-isoquinolyl)adenosine,
8-[2-(4-Morpholinyl)ethylamino]adenosine,
8-(2-Cyclohexylethylamino)adenosine,
8-(2(R,S)-Phenylpropylamino)adenosine,
8-[2-(4-Methylphenyl)ethylamino]adenosine,
8-[2-(1-methyl-2-pyrrolyl)ethylamino]adenosine,
8-[2-(4-Aminosulphonylphenyl)ethylamino]adenosine,
8-(4-Phenyl-1-piperazinyl)adenosine,
8-(1-Naphthylmethylamino)adenosine,
8-[2-(4-Hydroxyphenyl)ethylamino]adenosine,
8-(4-Phenylbutylamino)adenosine,
8-[2-(4-Chlorophenyl)ethylamino]adenosine,
8-[2-(2,4-Dichlorophenyl)ethylamino]adenosine,
8-(2-Propenylamino)adenosine,
8-(1(R)-Methyl-2-phenylethylamino)adenosine,
8-(4-Fluorobenzylamino)adenosine,
8-[(4-Hydroxycarbonyl)benzylamino]adenosine,
8-(2-propynylamino)adenosine,
8-[(4-trifluoromethyl)benzylamino]adenosine,
8-[(2,5-Dimethoxy)benzylamino]adenosine,
8-[2-(2-Thienyl)ethylamino]adenosine,
8-[2-(4-Aminophenyl)ethylamino]adenosine,
8-(2-Phenoxyethynylamino)adenosine,
8-[(2-Thienyl)methylamino)adenosine,
8-[(4-tert-Butyl)benzylamino]adenosine,
8-(1(R)-Phenylethylamino)adenosine,
8-(1(S)-Phenylethylamino)adenosine,
8-(6-Phenylhexylamino)adenosine,
8-[2-Hydroxy-1(S)-phenyl)ethylamino]adenosine,
2′-Deoxy-8-(2-phenylethylamino)adenosine,
2′-Deoxy-8-(3-phenylpropylamino)adenosine,
8-Benzylamino-2′-deoxyadenosine,
2′-Deoxy-8-(4-phenylbutylamino)adenosine,
2′-Deoxy-8-(6-phenylhexylamino)adenosine,
8-Ethoxyadenosine,
9-(β-D-Ribofuranosyl)-6-(3-thienyl)purine,
9-(β-D-Ribofuranosyl)-6-(1-thianthrenyl)purine,
6-(4-Biphenylyl)-9-(β-D-ribofuranosyl)purine,
6-(4-Methylthiophenyl)-9-(β-D-ribofuranosyl)purine,
6-(9-Phenanthrenyl)-9-(β-D-ribofuranosyl)purine,
9-(β-D-Ribofuranosyl)-6-(3-trifluoromethylphenyl)purine,
6-(2-Phenoxyphenyl)-9-(β-D-ribofuranosyl)purine,
6-(4-tert-Butylphenyl)-9-(β-D-ribofuranosyl)purine,
9-(β-D-Ribofuranosyl)-6-(2-trifluoromethoxyphenyl)purine,
6-(4-Phenoxyphenyl)-9-(β-D-ribofuranosyl)purine,
6-(2-Naphthyl)-9-(β-D-ribofuranosyl)purine,
6-(3-Biphenylyl)-9-(β-D-ribofuranosyl)purine,
6-[4-(2-Methylpropyl)phenyl]-9-(β-D-ribofuranosyl)purine,
6-(3-Fluorophenyl)-9-(β-D-ribofuranosyl)purine,
9-(β-D-Ribofuranosyl)-6-(4-trifluoromethylphenyl)purine,
6-(3-Ethoxyphenyl)-9-(β-D-ribofuranosyl)purine,
6-[3-(1-Methyl)ethylphenyl]-9-(β-D-ribofuranosyl)purine,
9-(β-D-Ribofuranosyl)-6-(4-trifluoromethoxyphenyl)purine,
6-(4-Ethylphenyl)-9-(β-D-ribofuranosyl)purine,
5-Fluoro-4-methylthio-1-(β-D-ribofuranosyl)pyrimidin-2(1H)-one,
5-Methyl-4-methylthio-1-(β-D-ribofuranosyl)pyrimidin-2(1H)-one,
2′,3′-Dideoxy-5-ethyl-3′-methoxyuridine,
4-(1-Pyrrolyl)-1-(β-D-ribofuranosyl)pyrimidin-2(1H)-one,
4-Oximino-1-(β-L-ribofuranosyl)pyrimidin-2(1H)-one,
5-Fluoro-4-oximino-1-(β-D-ribofuranosyl)pyrimidin-2(1H)-one; and
the hydrolyzable esters, hydrolyzable ethers and pharmaceutically acceptable salts thereof.
